(19) United States
(12) Patent Application Publication
(10) Pub. No.: US 2010/0222319 A1
(43) Pub. Date: Sep. 2, 2010

(54) NICOTINAMIDE DERIVATIVES, PREPARATION THEREOF AND THERAPEUTIC USE THEREOF

(75) Inventors: Claude BERNHART, Saint Gely du Fesc (FR); Monsif BOUABOULA, Juvignac (FR); Pierre CASELLAS, Castelnau-le-lez (FR); Samir JEGHAM, Montferrier-sur-lez (FR); Jèrome ARIGON, Saint-Gely-du-Fesc (FR); Romain COMBET, Montpellier (FR); Sandrine HILAIRET, Saint Andre de Sangonis (FR); Pierre FRAISSE, Juvignac (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/732,749

(22) Filed: Mar. 26, 2010

Related U.S. Application Data
(63) Continuation of application No. PCT/FR2008/001338, filed on Sep. 26, 2008.

(30) Foreign Application Priority Data
Sep. 28, 2007 (FR) 0706799

(51) Int. Cl. A61K 31/4545; A61P 35/00; C07F 5/02; C07D 401/12; A61K 31/444; C07D 401/14; A61K 31/496; C07D 417/14; A61K 31/541; C07D 413/14; A61K 31/5377; A61K 31/55

(52) U.S. Cl. 514/210.2; 546/13; 546/262; 514/332; 546/194; 514/318; 544/364; 514/253.13; 544/58.2; 514/227.8; 544/131; 514/237.2; 540/597; 514/217.04

(57) ABSTRACT

The disclosure relates to compounds of formula (I):

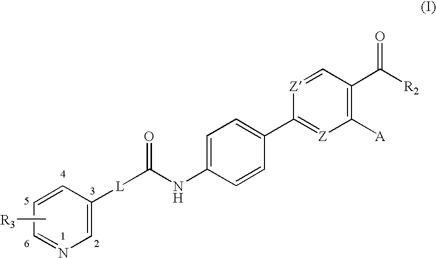

wherein A, Z, Z′, L, R₂ and R₃ are as defined in the disclosure, to compositions comprising said compounds, and to methods for the manufacture and therapeutic use thereof.

The present invention relates to nicotinamide derivatives, to the compositions comprising them and to their therapeutic application, in particular as anticancers. The invention also relates to the process for the preparation of these compounds and to some of the intermediates.

### BACKGROUND

United States application US 2006/0216288 describes anticancer compounds of general formula:

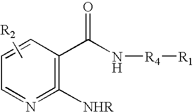

in which the substituent R₂ can in particular be a hydrogen atom, a hydroxyl or amino group, an alkyl or alkynyl group or an optionally substituted phenyl group.

International application WO 2006/028958 describes anticancer compounds of general formula:

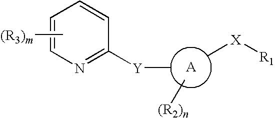

in which A denotes a carbocycle or heterocycle.

United States application US 2004/0067985 describes antiangiogenesis compounds of general formula:

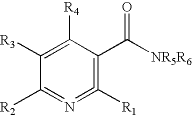

in which R₂ can in particular be an aryl or alkyl group.

International application WO 03/068747 describes compounds which are inhibitors of enzyme P38 of general formula:

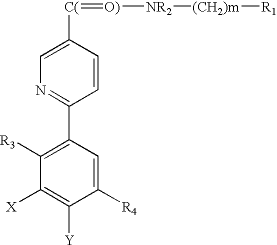

International application WO 2005/003099 describes compounds of general formula:

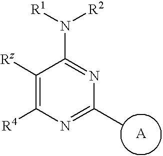

in which A can represent a phenyl group which comprises the —NR₁R₂ group.

International application WO 2007/031829 describes compounds of general formula:

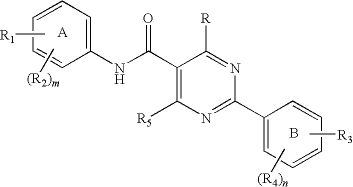

The specific group

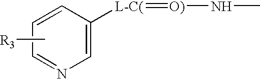

of the compounds of the invention is neither described nor suggested in any of these patent applications.

International application WO 2005/051366 describes anticancer compounds of general formula:

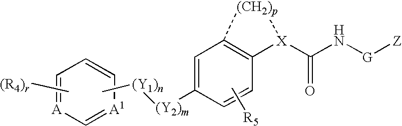

in which Z represents a phenyl or indanyl group and not a pyridinyl group.

International application WO 97/48397 describes anticancer compounds of general formula:

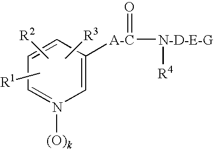

in which E represents a heterocycle comprising a nitrogen atom and optionally an oxygen atom.

International application WO 2007/016538 describes compounds of general formula:

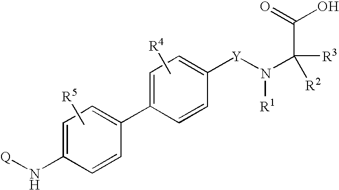

in which Q can represent an R₁₃—NR₁₂—C(═O)— group, it being possible for R₁₃ to be a 2-, 3- or 4-pyridinyl group, R₄ and R₅ representing a hydrogen atom, an alkyl, alkoxy, —OH, —CF₃ or —CN group. These compounds are used in the treatment of obesity.

International application WO 00/35864 describes compounds of general formula:

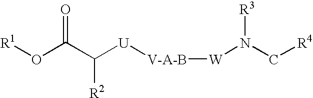

in which A and B can each be a 1,3- or 1,4-para-phenylene or 2,4- or 2,5-thienylene group, V represents an alkylene or NR₂CO or NR₂SO₂ group, and U represents an alkylene group or a single bond. The ring A can be substituted, more particularly by alkoxy groups or by a halogen atom. These compounds all comprise the —CHR₂COOR₁ unit, which the compounds of the invention do not comprise. Furthermore, the compounds of the invention are characterized by the presence on the ZZ′ ring of the substituents A and COR₂, which is not described in WO 00/35864.

### DESCRIPTION OF THE INVENTION

### Definitions Used

In the context of the present invention, and unless otherwise mentioned in the text:
a halogen atom is understood to mean: a fluorine, chlorine, bromine or iodine atom;
an alkyl group is understood to mean: a saturated aliphatic hydrocarbon group comprising from 1 to 6 carbon atoms (advantageously from 1 to 4 carbon atoms) which is linear or, when the alkyl chain comprises at least 3 carbon atoms, branched or cyclic. Mention may be made, by way of examples, of the methyl, ethyl, propyl, isopropyl, butyl, isobutyl, tert-butyl, methylcyclopropyl, pentyl, 2,2-dimethylpropyl, hexyl, cyclopropyl, cyclobutyl, cyclopentyl or cyclohexyl groups;
an alkoxy group is understood to mean: an —O-alkyl group, where the alkyl group is as defined above;
a heteroatom is understood to mean: a nitrogen, oxygen or sulphur atom;
a cycloalkyl group is understood to mean: a cyclic alkyl group comprising between 3 and 8 carbon atoms, all the carbon atoms being involved in the cyclic structure. Mention may be made, by way of examples, of the cyclopropyl, cyclobutyl, cyclopentyl or cyclohexyl groups;
an aryl group is understood to mean: a monocyclic aromatic group, for example a phenyl group;
a heteroaryl group is understood to mean: a monocyclic aromatic group comprising one or more heteroatom(s) involved in the cyclic structure. Mention may be made, by way of examples, of the pyridine group;
a heterocycloalkyl group is understood to mean: a cycloalkyl group as defined above initially comprising from 1 to 4 heteroatoms involved in a cyclic structure. Mention may be made, by way of examples, of the tetrahydrofuranyl, azetidinyl, pyrrolidinyl, piperidinyl, N—[(C₁-C₄)alkyl]piperidinyl, morpholinyl, piperazinyl, azepanyl, thiomorpholinyl, 1-oxothiomorpholinyl or 1,1-dioxothiomorpholinyl groups.

According to a 1^{st} aspect, a subject-matter of the present invention is a compound of formula (I):

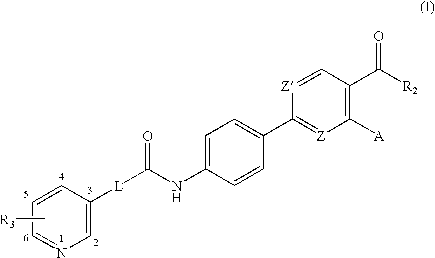

in which:
A represents an —NR₁R′₁ or (C₁-C₆)alkoxy group;
Z and Z′ respectively represent N and CH; N and CF; N and N; CH and CH; CH and N;
L represents a —CH═CH— or —CH₂CH₂— or —(CH₂)ₙ—Y— group in which the Y group (attached to the C═O) represents an oxygen atom or an —NH— group and n is an integer ranging from 1 to 4;
R₁ and R′₁ are such that:
   (i) R₁ represents:
      a hydrogen atom;
      an aryl group optionally substituted by one or more halogen atom(s);
      a heteroaryl group;
      a (C₃-C₆)cycloalkyl group;
      a (C₁-C₆)alkyl group, optionally substituted by:
         one or more hydroxyl or (C₁-C₆)alkoxy, preferably (C₁-C₄)alkoxy, group(s);
         an aryl group;
         a (C₃-C₆)cycloalkyl group;
         a heteroaryl group;
         a heterocycloalkyl group;
         an —NRₐR_{b} group in which Rₐ and R_{b} represent, independently of one another, a hydrogen atom or a (C₁-C₆)alkyl, preferably (C₁-C₄)alkyl, group or form, together with the nitrogen atom to which they are connected, a heterocycloalkyl group optionally comprising another nitrogen atom;
         and R′₁ represents a hydrogen atom or a (C₁-C₆)alkyl group;
or
   (ii) R₁ and R′₁ form, together with the nitrogen atom to which they are connected, a heterocycloalkyl group;
R₂ represents a -Q-R₄ group;
Q represents an oxygen atom or the —NH— group;
R₄ represents:
   a hydrogen atom;
   a heteroaryl group;
   a (C₃-C₆)cycloalkyl group;
   a (C₁-C₆)alkyl group, optionally substituted by:
      one or more hydroxyl or (C₁-C₆)alkoxy, preferably (C₁-C₄)alkoxy, groups;
      a heteroaryl group;
      a heterocycloalkyl group;
      an —NR_{c}R_{d} group in which R_{c} and R_{d} represent, independently of one another, a hydrogen atom or a (C₁-C₆)alkyl group or form, together with the nitrogen atom to which they are connected, a heterocycloalkyl group optionally comprising, in the ring, another heteroatom, such as a nitrogen or oxygen atom or the —S(O)_{q} group, with q=0, 1 or 2, and optionally being substituted by one or more substituent(s), which are identical to or different from one another when there are several of them, chosen from a halogen atom or an —OH; (C₁-C₄)alkoxy or (C₁-C₄)alkyl group;
R₃ represents at least one substituent of the pyridine ring chosen from a hydrogen or fluorine atom or a (C₁-C₄)alkyl, (C₁-C₄)alkoxy, —OH, —CN or —NRₑR_{f} group in which Rₑ and R_{f} represent a hydrogen atom or a (C₁-C₄)alkyl group or else Rₑ represents a hydrogen atom and R_{f} represents a (C₁-C₄)alkyl, —C(═O)O(C₁-C₄)alkyl or —C(═O)(C₁-C₄)alkyl group.

A can represent an —NR₁R′₁ group in which:

### (i) R₁ can be:

a hydrogen atom;
an aryl group optionally substituted by one or more halogen atom(s) (preferably a fluorine atom). The aryl group can be the phenyl group;
a heteroaryl group, such as, for example the 3- or 4-pyridinyl group;
a (C₃-C₆)cycloalkyl group, such as, for example, the cyclopropyl, cyclobutyl, cyclopentyl, cyclohexyl group;
a (C₁-C₆)alkyl, optionally substituted by:
   one or more —OH or (C₁-C₆)alkoxy, preferably (C₁-C₄)alkoxy, group(s): for example methoxy;
   an aryl group: for example, the phenyl group;
   a (C₃-C₆)cycloalkyl group: for example, the cyclopropyl group;
   a heteroaryl group: for example, the pyridinyl group, in particular 2-, 3- or 4-pyridinyl group;
   a heterocycloalkyl group: for example, the 2-tetrahydrofuryl group;
   an —NRₐR_{b} group in which Rₐ and R_{b} represent, independently of one another, a hydrogen atom or a (C₁-C₆)alkyl, preferably (C₁-C₄)alkyl, group or form, together with the nitrogen atom to which they are connected, a heterocycloalkyl group optionally comprising, in the ring, another nitrogen atom. Rₐ and R_{b} can be two (C₁-C₆)alkyl groups, for example two methyl groups. The heterocycloalkyl formed by Rₐ and R_{b} can, for example be the pyrrolidinyl

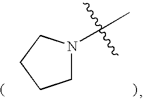

piperazinyl

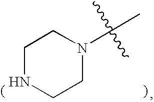

piperidinyl

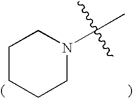

or N—[(C₁-C₄)alkyl]piperidinyl

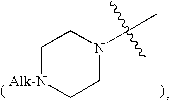

for example N-methylpiperidinyl, group.

R₁ can be chosen from one of those described in Table I.

and R′₁ represents a hydrogen atom or a (C₁-C₆)alkyl group. R′₁ can be chosen from one of those described in Table I. An R₁/R′₁ combination can also be chosen from one of those described in Table I.
(ii) R₁ and R′₁ form, together with the nitrogen atom to which they are connected, a heterocycloalkyl group, for example the pyrrolidinyl

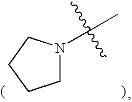

piperidinyl

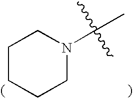

or azetidinyl

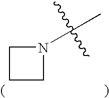

group.

A can also represent a (C₁-C₆)alkoxy group, for example the ethoxy group.

R₂ can represent an —NHR₄ group (Q=—NH—) in which R₄ represents:
a hydrogen atom;
a heteroaryl group, such as, for example, the pyridinyl group, in particular 2-, 3- or 4-pyridinyl group;
a (C₃-C₆)cycloalkyl group, such as, for example, the cyclopropyl or cyclopentyl group;
a (C₁-C₆)alkyl group, optionally substituted by:
   one or more —OH or (C₁-C₆)alkoxy, preferably (C₁-C₄)alkoxy group, for example methoxy;
   a heteroaryl group: for example the pyridinyl group, in particular 2-, 3- or 4-pyridinyl group;
   a heterocycloalkyl group: for example, the morpholinyl, pyrrolidinyl, piperazinyl, or piperidinyl group, more particularly by the 4-piperidinyle

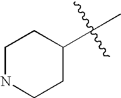

or 4-N—[(C₁-C₄)alkyl]piperidinyl

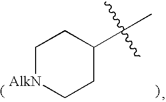

for example 4-N-methylpiperidinyl, group;
an —NR_{c}R_{d} group in which R_{c} and R_{d} represent, independently of one another, a hydrogen atom or a (C₁-C₆)alkyl group or form, together with the nitrogen atom to which they are connected, a heterocycloalkyl group optionally comprising, in the ring, another heteroatom, such as a nitrogen or oxygen atom or the —S(O)_{q} group, with q=0, 1 or 2.

The heterocycloalkyl group formed by R_{c} and R_{d} can, for example, be the pyrrolidinyl

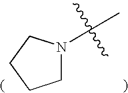

piperidinyl

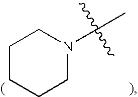

piperazinyl

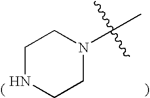

or N—[(C₁-C₄)alkyl]piperazinyl

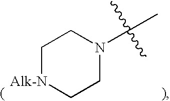

for example N-methyl- or N-propylpiperazinyl, azepanyl

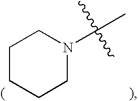

morpholinyl

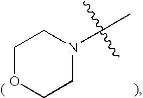

thiomorpholinyl

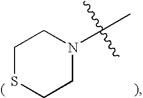

1-oxothiomorpholinyl

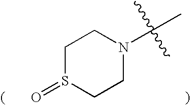

or 1,1-dioxothiomorpholinyl

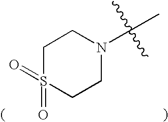

group.

The heterocycloalkyl group formed by R_{c} and R_{d} can optionally be substituted by one or more substituent(s), which are identical to or different from one another when there are several of them, chosen from: —OH; (C₁-C₄)alkoxy: for example methoxy; (C₁-C₄)alkyl: for example methyl; halogen atom: for example fluorine atom. Thus, the substituted heterocycloalkyl can be the 3-hydroxypiperidinyl

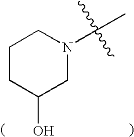

or 4-hydroxypiperidinyl

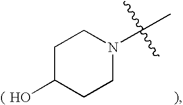

4,4′-difluoropiperidinyl

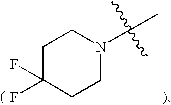

4-methoxypiperidinyl

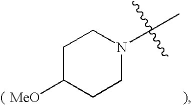

2-methylpyrrolidinyl

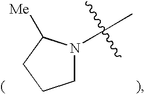

cis-2,6-dimethylmorpholinyl

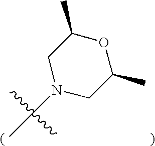

or 3-fluoropyrrolidinyl

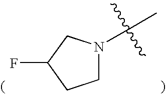

group.

R₂ can also represent an —OR₄ group (Q=—O—) in which R₄ represents a (C₁-C₄)alkyl group optionally substituted by the preceding —NR_{c}R_{d} group. It can, for example, be the piperidinyl group

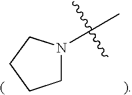

R₂ or R₄ can be chosen from one of those described in Table I.

A pyridine ring can comprise from 1 to 4 R₃ substituents chosen from a hydrogen or fluorine atom or a (C₁-C₄)alkyl, (C₁-C₄)alkoxy, —OH, —CN or —NRₑR_{f} group in which Rₑ and R_{f} represent a hydrogen atom or a (C₁-C₄)alkyl group or else Rₑ represents a hydrogen atom and R_{f} represents a (C₁-C₄)alkyl, —C(═O)(C₁-C₄)alkyl or —C(═O)(C₁-C₄)alkyl group. R₃ can be chosen from those described in Table I.

Preferably, R₃ is in the 5 or 6 position on the pyridine ring (the L group being in the 3 position on this ring), as represented below:

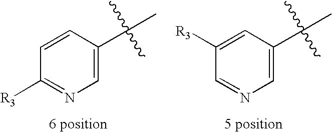

R₃ is more preferably still in the 6 position. Preferably R₃ represents a hydrogen atom or 5- or 6-NH₂. When R₃ represents the —OH group in the 2 or 6 position (cf. compound No. 123), the pyridine ring also exists in the 2-pyridone form:

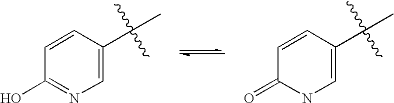

case of the —OH group in the 6 position

L represents a —CH═CH—, —CH₂CH₂— or —(CH₂)ₙ—Y— group in which the Y group (attached to the C═O) represents an oxygen atom or an —NH— group and n is an integer ranging from 1 to 4. L can be one of those described in Table I. Preferably, L represents the —CH₂—NH—, —CH₂—O— or —CH═CH— group. Preference is also given, in the case where L represents the —CH═CH— group, to the E isomers rather than the Z isomers.

The ring comprising Z and Z′ can be one of the following rings:

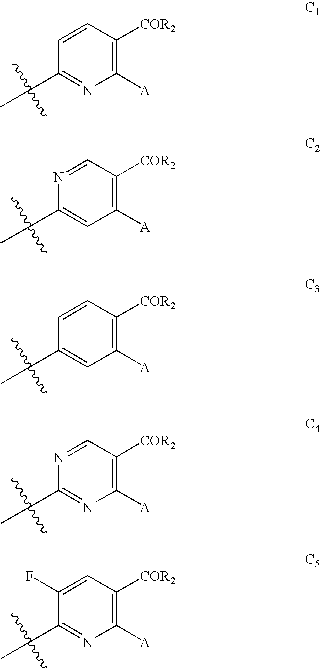

According to a 1^{st} combination,
R₁ and R′₁ represent, independently of one another, a hydrogen atom or a (C₁-C₆)alkyl group;
Q represents the —NH— group;
R₄ represents a hydrogen atom or a (C₁-C₆)alkyl group.

More particularly, R₁ represents a (C₁-C₆)alkyl group and R′₁ represents a hydrogen atom or else R₁ and R′₁ represent two (C₁-C₆)alkyl groups.

According to a 2^{nd} combination,
R₁ and R′₁ represent, independently of one another, a hydrogen atom or a (C₁-C₆)alkyl group;
Q represents the —NH— group;
R₄ represents a (C₁-C₆)alkyl group substituted by:
   one or more —OH or (C₁-C₆)alkoxy, preferably (C₁-C₄)alkoxy, groups;
   the —NR_{c}R_{d} group in which R_{c} and R_{d} represent, independently of one another, a hydrogen atom or a (C₁-C₆)alkyl group or form, together with the nitrogen atom to which they are connected, a heterocycloalkyl group chosen from a pyrrolidinyl, piperidinyl, piperazinyl or N—[(C₁-C₄)alkyl]piperazinyl, azepanyl, morpholinyl, thiomorpholinyl, 1-oxothiomorpholinyl, 1,1-dioxothiomorpholinyl, 3- or 4-hydroxypiperidinyl, 4,4′-difluoropiperidinyl, 4-methoxypiperidinyl, 2-methylpyrrolidinyl, cis-2,6-dimethylmorpholinyl or 3-fluoropyrrolidinyl group.

According to a 3^{rd} combination,
R₁ represents a (C₁-C₆)alkyl group substituted by:
   one or more —OH or (C₁-C₆)alkoxy, preferably (C₁-C₄)alkoxy, group(s);
   an —NRₐR_{b} group in which Rₐ and R_{b} represent, independently of one another, a hydrogen atom or a (C₁-C₆)alkyl, preferably (C₁-C₄)alkyl, group or form, together with the nitrogen atom to which they are connected, a heterocycloalkyl group chosen from a pyrrolidinyl, piperazinyl, piperidinyl or N—[(C₁-C₄)alkyl]piperidinyl group;
R′₁ represents a hydrogen atom;
Q represents the —NH— group;
R₄ represents a (C₁-C₆)alkyl group.

Rₐ and R_{b} can be identical and both represent a hydrogen atom or a (C₁-C₆)alkyl group or else can be different and represent a hydrogen atom and a (C₁-C₆)alkyl group.

According to a 4^{th} combination,
R₁ represents a (C₁-C₆)alkyl group substituted by a phenyl or 2-, 3- or 4-pyridinyl group;
R′₁ represents a hydrogen atom;
Q represents the —NH— group;
R₄ represents a (C₁-C₆)alkyl group.

According to a 5^{th} combination,
R₁ represents a (C₃-C₆)cycloalkyl group;
R′₁ represents a hydrogen atom;
Q represents the —NH— group;
R₄ represents a (C₁-C₆)alkyl group or a (C₃-C₆)cycloalkyl group.

R₁ can be the cyclopropyl, cyclobutyl, cyclopentyl or cyclohexyl group. R₄ can be the cyclopropyl or cyclopentyl group.

According to a 6^{th} combination,
R₁ represents a phenyl or 3- or 4-pyridinyl group;
R′₁ represents a hydrogen atom;
Q represents the —NH— group;
R₄ represents a (C₁-C₆)alkyl group.

According to a 7^{th} combination,
R₁ represents a phenyl group optionally substituted by one or more halogen atom(s);
R′₁ represents a hydrogen atom;
Q represents the —NH— group;
R₄ represents a (C₁-C₆)alkyl group optionally substituted by the —NR_{c}R_{d} group in which R_{c} and R_{d} form, together with the nitrogen atom to which they are connected, a heterocycloalkyl group chosen from the pyrrolidinyl or piperidinyl group.

According to an 8^{th} combination,
R₁ and R′₁ represent, independently of one another, a hydrogen atom or a (C₁-C₆)alkyl group;
Q represents the —NH— group;
R₄ represents a (C₁-C₆)alkyl group substituted by the 2-, 3- or 4-pyridinyl group.

The subgroup of compounds of formula (I′):

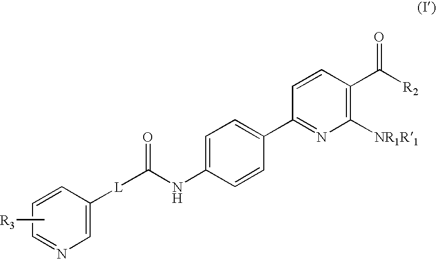

in which R₁, R′₁, R₂, R₃ and L are as defined above, in particular according to one of the combinations 1 to 8, is distinguished. More particularly, L represents the —(CH₂)ₙ—Y— group in which n is an integer ranging from 1 to 4 (n=1, 2, 3 or 4) and Y represents an oxygen atom or an NH group. More particularly, L represents the —CH₂NH— group.

The subgroup of compounds of formula (I″):

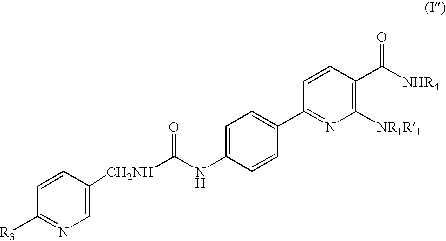

in which R₁, R′₁, R₄ are as defined above, in particular according to one of the combinations 1 to 8, is also distinguished.

Mention may be made, among the compounds which are the subject-matter of the invention, of those of Table I.

The compounds of the invention can exist in the form of bases or of addition salts with acids. Such addition salts also come within the invention. These salts are advantageously prepared with pharmaceutically acceptable acids but the salts of other acids of use, for example, in the purification of the isolation of the compounds also come within the invention. The compounds according to the invention can also exist in the form of hydrates or solvates, namely in the form of combinations or associations with one or more molecules of water or with a solvent. Such hydrates and solvates also come within the invention.

The compounds can comprise one or more asymmetric carbon atoms. They can also exist in the form of an enantiomers or diastereoisomers. These enantiomers or diastereoisomers and their mixtures come within the invention.

According to 2^{nd} aspect, the subject-matter of the invention is the process for preparation of the compounds of the invention and some of the reaction intermediates.

Preparation of the Compounds of Formula (I) for which L=-(CH₂)ₙY and R₂═NHR₄

These compounds can be prepared according to one of the following schemes 1-3.

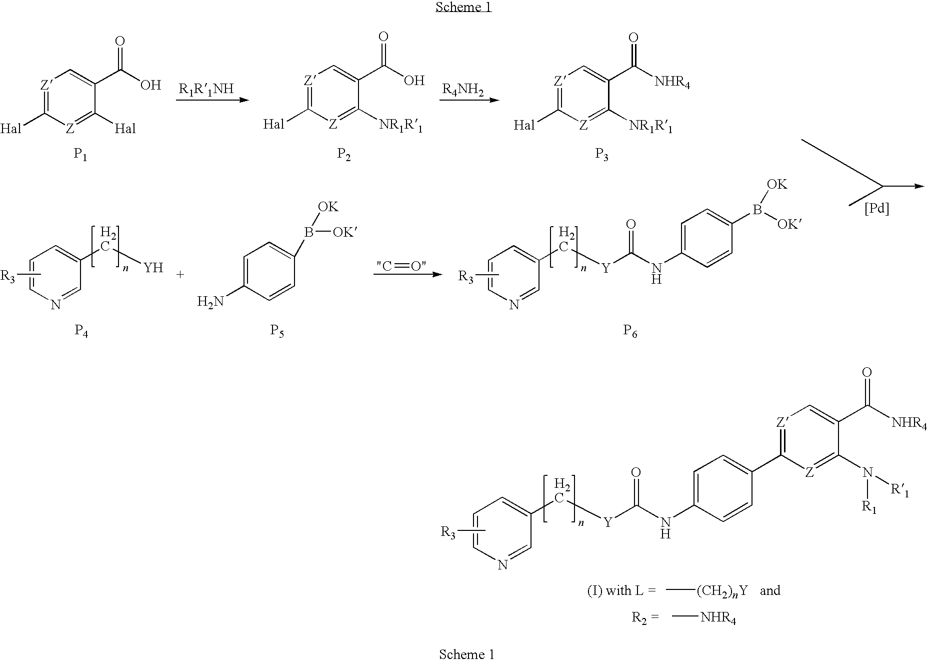

A coupling of Suzuki type of P₃ and P₆ is carried out. Hal represents the halogen atom (chlorine, bromine, iodine). The coupling is carried out in the presence of a palladium (in the (0) or (II) oxidation state) complex, such as, for example, Pd(PPh₃)₄, PdCl₂(PPh₃)₂, Pd(OAc)₂ or PdCl₂(dppf) or bis[di(tert-butyl)(4-dimethylaminophenyl)phosphine]dichloropalladium(II). The most frequently used complexes are palladium(0) complexes. The coupling is promoted in the presence of a base, which can, for example, be K₂CO₃, NaHCO₃, Et₃N, K₃PO₄, Ba(OH)₂, NaOH, KF, CsF, Cs₂CO₃, and the like. The coupling can be carried out in a mixture of an ethereal solvent and of an alcohol, for example a dimethoxyethane/ethanol mixture; the mixture can also be a toluene/water mixture (see ex. 19). The temperature can be between 50 and 100° C.

Further details with regard to Suzuki coupling, with regard to the operating conditions and with regard to the palladium complexes which can be used will be found in: N. Miyaura and A. Suzuki, Chem. Rev. (1995), 95, 2457-2483; A. Suzuki in Metal-catalyzed cross-coupling reactions, edited by Diederich, F. and Stang, P. J., Wiley-VCH: Weinheim, Germany, 1998, chapter 2, 49-97; and Littke, A. and Fu, G., Angew. Chem. Int. Ed. (1999), 38, 3387-3388.

K and K′ represent a hydrogen atom or an alkyl or aryl group, optionally connected to one another in order to form, together with the boron atom and the two oxygen atoms, a 5- to 7-membered ring. Use will be made, for example, of one of the following groups:

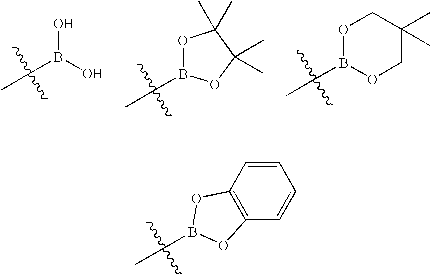

P₂ is obtained from the acid P₁ by monosubstitution in 2 position with an amine of formula R₁R′₁NH. In the case where Z and Z′ respectively represent N and CH, P₁ is a 2,6-dihalonicotinic acid, for example 2,6-dichloronicotinic acid, which is commercially available (cf. ex. 1). The reaction can take place at ambient temperature and in a protic solvent, such as an alcohol or water.

In the case where Z and Z′ both represent N and Hal represents a chlorine atom, P₂ is obtained from 2,4-dihydroxypyrimidine-5-carboxylic acid (cf. ex. 11).

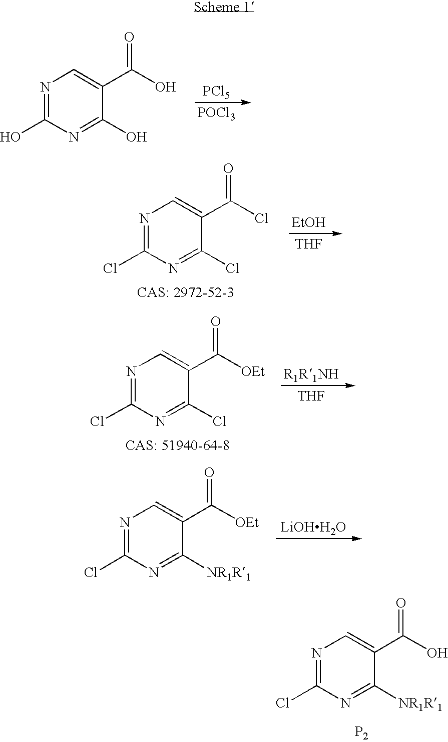

P₃ is prepared by amidation by reacting P₂ with an excess of amine R₄NH₂. Use may advantageously be made of an acid activator (coupling agent), such as, for example (benzotriazol-1-yloxy)tris(dimethylamino)-phosphonium hexafluorophosphate (or BOP, CAS: 56602-33-6, see also B. Castro. and Dormoy, J. R. Tetrahedron Letters, 1975, 16, 1219). The reaction is preferably carried out in the presence of a base (such as triethylamine) at ambient temperature in a solvent, such as tetrahydrofuran (THF) or dimethylformamide (DMF).

P₆ is prepared by reacting P₄ and P₅ in the presence of an agent which makes it possible to introduce the “C═O” unit (for example phosgene, triphosgene or N,N′-disuccinimidyl carbonate DSC). Advantageously, the reaction is carried out in the presence of triphosgene. It is also preferably carried out in the presence of a base, such as, for example triethylamine, and at a temperature of between −5° C. and ambient temperature in an ethereal solvent, such as THF. 4-(4,4,5,5-tetramethyl-1,3,2-dioxaborolan-2-yl)phenylamine has frequently been used for P₅. Example 8.1 presents an illustrative procedure for this reaction.

P₄ may be either commercially available or prepared according to methods known to the person skilled in the art. For example, the compounds 3-picolylamine (CAS No. 3731-52-0), 3-(2-aminoethyl)pyridine (CAS No. 20173-24-4), 3-pyridinemethanol (CAS No. 100-55-0), 5-aminoethyl 2-pyridinecarbonitrile (CAS No. 181130-14-3), 2-amino-5-aminomethylpyridine (CAS No. 156973-09-0), 2-fluoro-3-aminomethylpyridine (CAS No. 205744-16-7), 2,5,6-trifluoro-3-(aminomethyl)pyridine (CAS No. 771585-56-0), 2-methyl-5-aminomethylpyridine (CAS No. 56622-54-9), 3-methyl-5-aminomethylpyridine (CAS No. 771574-45-9), 2-methoxy-3-aminoethylpyridine (CAS No. 354824-19-4), 5-aminoethyl-1H-pyridin-2-one (CAS No. 131052-84-1) and 2-(BOC-amino)-5-(aminomethyl)pyridine (CAS No. 187237-37-2) are commercial products. 2-amino-5-aminomethylpyridine can also be prepared according to EP 0607804. 2-amino-5-aminomethylpyridine and 6-amino-3-aminomethyl-5-methylpyridine can be prepared according to preparations D and F of EP 1050534. 2-fluoro-5-aminomethylpyridine (CAS No. 205744-17-8) can be prepared according to Chinese Journal of Chemistry, 2006, 24(4), 521-526. 5-aminomethyl-2-(dimethylamino)pyridine (CAS No. 354824-17-2) is commercially available or can be prepared according to Journal of Agricultural and Food Chemistry, 2008, 56(1), 204-212. 3-fluoro-5-aminomethylpyridine (CAS No. 23586-96-1) and 2-fluoro-3-aminomethylpyridine can be prepared according to WO 2005066126 (preparations 46 and 47). 2-amino-3-methyl-5-aminomethylpyridine (CAS No. 187163-76-4) can be obtained by catalytic hydrogenation of the compound 6-amino-5-methylpyridinecarbonitrile (CAS No. 183428-91-3), the amine functional group being doubly protected with BOC. Likewise, the catalytic hydrogenation of N-(5-cyano-2-pyridinyl)acetamide (CAS No. 100130-61-8) and N-(5-cyano-2-pyridinyl)isobutyramide makes it possible to obtain the aminomethyl equivalents. Catalytic hydrogenation of 6-isopropylaminonicotinonitrile (CAS No. 160017-00-5) and 6-ethylamino-3-pyridinecarbonitrile (CAS No. 1016813-34-5) likewise produces the aminomethyl equivalents. Catalytic hydrogenation of 6-methylamino-3-pyridinecarbonitrile (CAS No. 261715-36-0) makes it possible to access 2-methylamino-5-aminomethylpyridine.

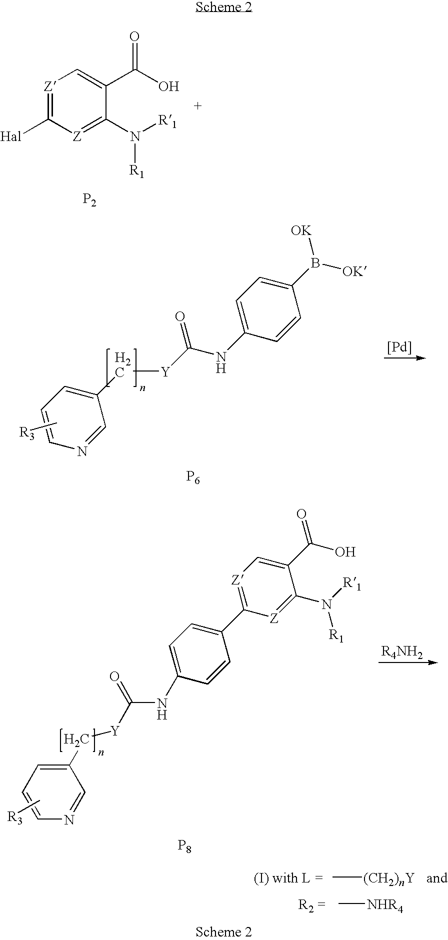

In Scheme 2, the Suzuki coupling (as described above) between P₂ (for example, Hal=Cl when Z and Z′ respectively represent N and CH) and P₆ is first carried out in order to result in P₈ and then the R₄ group is introduced by reacting the acid functional group of P₈ with an excess of amine R₄NH₂ (amidation). An acid activator, such as, for example, BOP, is advantageously used to activate the reaction. In the case where R₄ represents a pyridine group (cf. compounds No. 67 and 68), the activator can, for example, be EDCl (1-(3-dimethylaminopropyl)-3-ethylcarbodiimide hydrochloride).

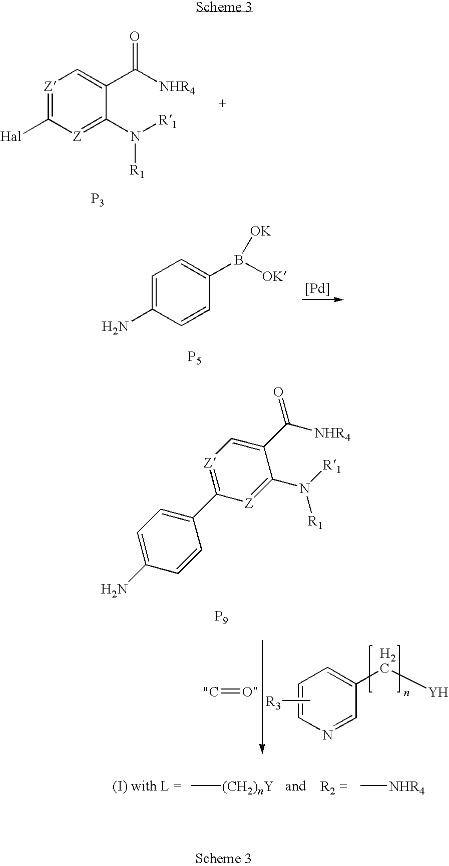

In Scheme 3, the Suzuki coupling of P₃ and P₅ is carried out in order to give P₉ and then P₉ and P₄ are reacted in the presence of an agent which makes it possible to introduce the “C═O” unit and optionally of a base, such as triethylamine. The reaction is carried out in an ethereal solvent, such as THF, preferably at an ambient temperature. Preferably, DSC is used to introduce the “C═O” unit.

Preparation of the Compounds of Formula (I) for which L=—(CH₂)ₙY— and R₂=—OR₄

According to an alternative form of Scheme 2, these compounds are prepared by esterification of P₈ and of R₄OH (Scheme 2′).

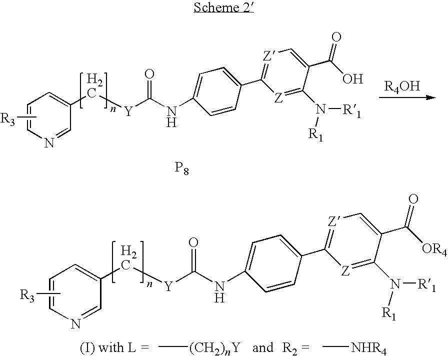

According to an alternative form of Scheme 3, it is also possible to use P′₃ in place of P₃. P′₃ is obtained by esterification of P₂ and of R₄OH (Scheme 3′):

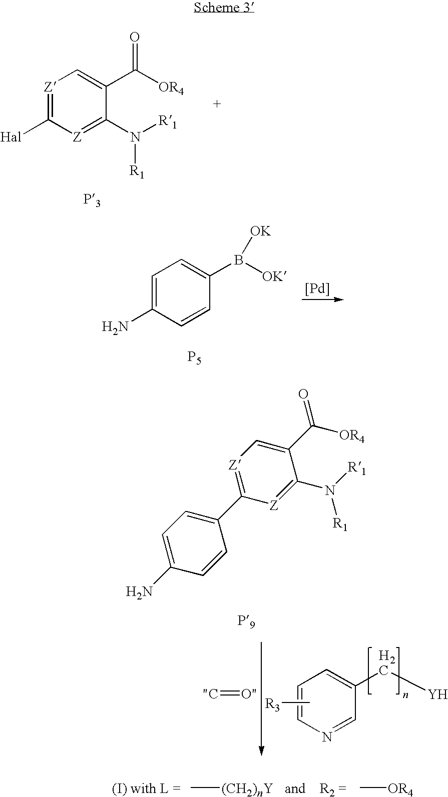

Esterification is known to a person skilled in the art and consists in reacting the acid functional group of P₂ or P₈ with the alcohol R₄OH in the optional presence of a strong acid as catalyst (cf. Practical Organic Chemistry, A. I. Vogel, 3^{rd} ed., page 382) or of an acid activator, such as EDCl.

Preparation of the compounds of formula (I) for which L=—CH═CH— and R₂═NHR₄

These compounds are obtained by coupling of Suzuki type of P₃ (for example, Hal=Cl when Z and Z′ respectively represent N and CH) and of P₁₁. P₁₁ is obtained by an amidation between P₅ and P₁₀. The amidation can advantageously be carried out in the presence of an acid activator, such as, for example, BOP.

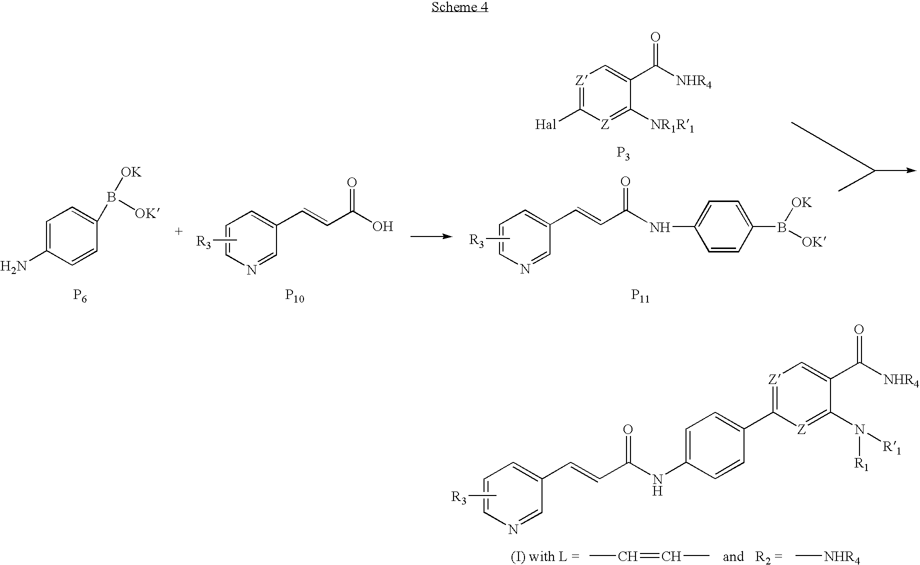

P₁₀ may either be commercially available or be prepared according to methods known to a person skilled in the art. For example, trans-3-(3-pyridyl)acrylic acid is sold by Sigma-Aldrich. P₁₀ can also be prepared according to J. Org. Chem., 1998, 63, 8785-8789, from the corresponding β-formylpyridine.

According to Scheme 5, P₁₀ is reacted with P₉, advantageously in the presence of an acid activator, such as, for example, BOP.

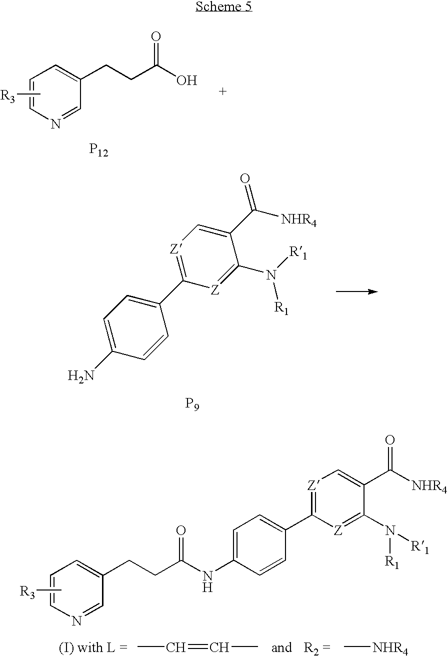

Preparation of the Compounds of Formula (I) for which L=—CH₂CH₂— and R₂═NHR₄

For these compounds, use may be made of the preceding Scheme 4 using P₁₂ in place of P₁₀:

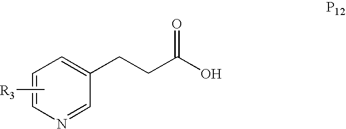

P₁₂ may either be commercially available or be prepared according to methods known to a person skilled in the art. For example, 3-(3-pyridinyl)propanoic acid is sold by Sigma-Aldrich. P₁₂ can also be prepared by hydrogenation of P₁₀ (Journal of Medicinal Chemistry, 1993, 36(22), 3293-9).

Use may also be made of P₁₂ in place of P₁₀ in the preceding Scheme 5.

Preparation of the Compounds of Formula (I) for which L=—CH═CH— or —CH₂CH₂— and R₂=—OR₄

P₁₁ and P′₃ (in place of P₃) are reacted in the preceding Scheme 4 in order to obtain compounds of formula (I) for which L=—CH═CH— and R₂=—OR₄. Likewise, starting from P′₃ and P₁₂, the compounds of formula (I) for which L=—CH₂CH₂— and R₂=—OR₄ are obtained.

The compounds for which A represents a (C₁-C₆)alkoxy group are obtained according to Schemes equivalent to the preceding Schemes starting from an equivalent compound P₁₃.

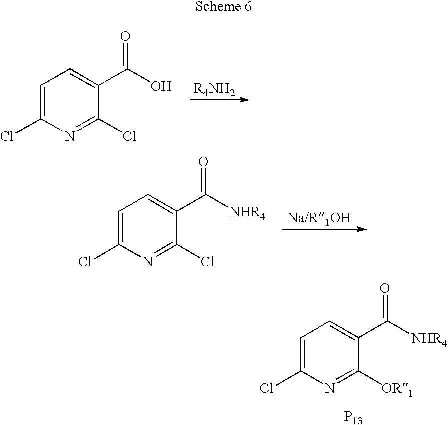

P₁₃ can be obtained according to Scheme 6. Amidation with R₄NH₂ can be carried out in the presence of an acid activator, such as, for example, N,N′-carbonyldiimidazol (CDI) (see in this connection: R. Paul and G. W. Anderson (1960), “N,N′-carbonyldiimidazole, a New Peptide Forming Reagent”, Journal of the American Chemical Society, 82: 4596-4600). The reaction can be carried out in a solvent such as THF. The conditions of Ex. 10.1 may act as a model. The following stage is carried out in the presence of the alkoxide R″₁O⁻. The reaction can be carried out in THF at a temperature of the order of 70° C. The conditions of Ex. 10.2 may act as a model.

### Protection of the Primary or Secondary Amine Functional Group

It may be necessary to use, in at least one of the stages, a protective group (PG) in order to protect one or more chemical functional group, in particular a primary or secondary amine functional group. For example, when R_{c} and R_{d} both represent a hydrogen atom, the amidation of Scheme 2 is carried out using, for R₄NH₂, the compound H₂N—(C₁-C₆)alkyl-NH-PG, where PG advantageously represents BOC (tert-butoxycarbonyl). Thus, for compound No. 32, the compound H₂N—(CH₂)₆—NHBOC was used for R₄NH₂. Likewise, when the heterocycloalkyl group formed by R_{c} and R_{d} represents the piperazinyl group

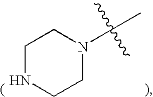

the —NH— functional group thereof can advantageously be protected. In this case, the following compound

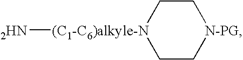

where PG advantageously represents BOC, is used. Likewise, when R₃ represents —NH₂ or —NH-alkyl, the —NH— functional group is preferably protected, advantageously using BOC (see, for example, compounds No. 81, 87, 93, 94 and 98), which makes it possible to increase the yield of desired product.

The functional group(s) is/are subsequently obtained by a stage of deprotection (final or intermediate), the conditions of which depend on the nature of the protected functional group(s) and protective group used. In the case of the protection of the —NH₂ or —NH-functional groups by BOC, the deprotection stage is carried out in an acid medium using, for example, HCl or triflic acid. If appropriate, the associated salt (hydrochloride or triflate) is thus obtained; see compounds No. 5, 32, 94, 104 or 119. Another method of obtaining the salts consists in bringing the compound into contact in its base form with the acid.

In the preceding Schemes, the starting compounds and the reactants, when their method of preparation is not described, are commercially available or described in the literature, or else can be prepared according to methods which are described therein or which are known to a person skilled in the art. A person skilled in the art can also draw as a model on the operating conditions given in the examples which are described below.

According to a 3^{rd} aspect, the invention relates to a pharmaceutical composition comprising a compound as defined above in combination with a pharmaceutically acceptable excipient. The excipient is chosen from the usual excipients known to a person skilled in the art according to the pharmaceutical form and the method of administration desired. The method of administration can, for example, be via the oral route or via the intravenous route.

According to a 4^{th} aspect, the subject-matter of the invention is a medicament which comprises a compound as defined above, and also the use of a compound as defined above in the manufacture of a medicament. It will be of use in treating a pathological condition, in particular cancer.

This medicament can have a therapeutic use, in particular in the treatment or the prevention of diseases caused or exacerbated by the proliferation of cells and in particular tumour cells.

The medicament (and also a compound according to the invention) can be administered in combination with one (or more) anticancers, in particular chosen from:
chemotherapy agents, such as alkylating agents, platinum derivatives, antibiotic agents, antimicrotubule agents, taxoids, anthracyclines, group I and II topoisomerase inhibitors, fluoropyrimidines, cytidine analogues, adenosine analogues, enzymes, and also oestrogenic and androgenic hormones;
antivascular or antiangiogenic agents.

It is also possible to combine a treatment by radiation. This treatment can be administered simultaneously, separately or else sequentially. The treatment will be adapted by the practitioner according to the patient and the tumour to be treated.

According to a 5^{th} aspect, the invention also relates to a method for the treatment of the pathologies indicated above which comprises the administration to a patient of an effective dose of a compound according to the invention or one of its pharmaceutically acceptable salts or its hydrates or its solvates.

### EXAMPLES

The following examples illustrate the preparation of some compounds in accordance with the invention. These examples are not limiting and serve only to illustrate the present invention. The numbers of the compounds exemplified refer to those given in the table below, in which the chemical structures and the physical properties of some compounds according to the invention are illustrated.

The compounds have been analyzed by HPLC-UV-MS coupling (liquid chromatography, ultraviolet (UV) detection and mass detection). The device used is composed of an Agilent chromatographic sequence equipped with an Agilent diode array detector and with a Waters ZQ single quadrupole mass spectrometer or a Waters Quattro-Micro triple quadrupole mass spectrometer.

### Mass Spectrometry Conditions

The liquid phase chromatography/mass spectrometer (LC/MS) spectra were recorded in positive electrospray (ESI) mode, in order to observe the ions resulting from the protonation of compounds analyzed (MH⁺) or from the formation of adducts with other cations, such as Na⁺, K⁺, and the like. The ionization parameters are as follows: cone voltage: 20 V; capillary voltage: 3 kV; source temperature: 120° C.; desolvation temperature: 450° C.; desolvation gas: N₂ at 450 I/h.

The HPLC conditions are chosen from one of the following methods:

| Conditions | A | B | C | D | E |
|---|---|---|---|---|---|
| Column | Symmetry C18 | Symmetry C18 | XTerra MS | Acquity BEH | XTerra C₁₈ |
| | (50 × 2.1 mm; | (50 × 2.1 mm; | C18 | C18 | (2.1 × 50 mm; |
| | 3.5 μm) | 3.5 μm) | (50 × 2.1 mm; | (50 × 2.1 mm; | 3.5 μm) |
| | | | 3.5 μm) | 1.7 μm) | No. 186000400 |
| Eluant A | H₂O + 0.005% | H₂O + 0.005% | AcONH₄ | H₂O + 0.05% | H₂O + 0.005% |
| | TFA at | TFA at | 10 mM at | TFA at | TFA |
| | approximately | approximately | pH ~7 | approximately | |
| | pH 3.1 | pH 3.1 | | pH 3.1/CH₃CN | |
| | | | | (97/3) | |
| Eluant B | CH₃CN + | CH₃CN + | CH₃CN | CH₃CN + | CH₃CN |
| | 0.005% TFA | 0.005% TFA | | 0.035% TFA | |
| Gradient | 100:0 (0 min) | 100:0 (0 min) | 100:0 (0 min) | 100:0 (0 min) | 95% of A to |
| | → 10:90 | → 10:90 | → 10:90 | → 5:95 | 90% of B in |
| | (10 min) → | (20 min) → | (10 min) → | (2.3 min) → | 17 min, then |
| | 100:0 (15 min) | 100:0 (30 min) | 100:0 (20 min) | 5:95 (2.9 min) | 90% of B for |
| | | | | → 100:0 | 5 min |
| | | | | (3 min) → | |
| | | | | 100:0 | |
| | | | | (3.5 min) | |
| T. column | 30° C. | 30° C. | 30° C. | 40° C. | Column not |
| | | | | | thermostatically |
| | | | | | controlled |
| Flow rate | 0.4 ml/min | 0.4 ml/min | 0.4 ml/min | 1 ml/min | 0.3 ml/min |
| Detection | λ = 220 nm | λ = 220 nm | λ = 220 nm | λ = 220 nm | λ = 220 nm |

TFA: trifluoroacetic acid

### Example 1

### 2-ethylamino-N-methyl-6-[4-(3-(pyridin-3-ylmethyl)ureido)phenyl]-nicotinamide (Compound No. 1)

### 1.1: 6-Chloro-2-(ethylamino)nicotinic acid

26.1 g (0.136M) of 2,6-dichloronicotinic acid are mixed in a round-bottomed flask with 180 ml of a 70% aqueous solution of ethylamine in water. The solution is stirred at ambient temperature for 5 days and then the solvent is evaporated under reduced pressure. The residue is taken up in 100 ml of water. The reaction medium is cooled with an ice bath and acidified to pH 3 with the 5N HCl solution. Finally, the precipitate is filtered off and washed with cold water in order to be finally dried under vacuum over P₂O₅ at 60° C. 24.93 g (yield yd=91.4%) of white solid are obtained. M.p. (melting point)=157-159° C.

### 1.2: 6-Chloro-2-ethylamino-N-methylnicotinamide

2.09 ml (15 mm) of triethylamine, 5 ml (10 mm) of a 2N solution of methylamine in THF and 2.06 g (5 mm) of BOP are successively added to a solution of 1.003 g (5 mm) of compound obtained in stage 1.1 in 40 ml of THF. The medium is stirred at ambient temperature for 18 h, followed by evaporation of the solvent under reduced pressure. The residue is taken up in ethyl acetate and then successively washed with water, a 3% solution of KHSO₄ in water, a 10% solution of Na₂CO₃ in water and a saturated NaCl solution. 1.06 g of nicotinamide are obtained. The yield is quantitative. (LC/MS; MH+ 214, retention time tᵣ=7.48 min).

### 1.3: 1-(Pyridin-3-ylmethyl)-3-[4-(4,4,5,5-tetramethyl-1,3,2-dioxaborolan-2-yl)phenyl]urea

57.2 ml (410.8 mm) of triethylamine are introduced dropwise into a mixture of 15 g (68.47 mm) of 4-(4,4,5,5-tetramethyl-1,3,2-dioxaborolan-2-yl)phenylamine and of 12.19 g (41.08 mm) of triphosgene in 15 l of THF cooled with an ice/water bath to a temperature of between 0° C. and 5° C. After stirring at a temperature of between 0° C. and 5° C. for 1 h, 8.29 g (76.68 mm) of 3-(aminomethyl)pyridine are added to a reaction medium. The mixture is stirred for 20 h while allowing the temperature to rise to ambient temperature. The THF is evaporated. The ratio is taken up in water and then extracted with ethyl acetate. The organic phase is subsequently dried over Na₂SO₄, filtered and evaporated. The residue is recrystallized from a minimum amount of ethyl acetate. 13 g (yd=53.8%) of white solid composed of 89% of the expected compound and 11% of the corresponding boronic acid are obtained (LC/MS; MH+ 354 and 272, tᵣ=6.25 and 3.65 min).

### 1.4: 2-Ethylamino-N-methyl-6-[4-(3-(pyridin-3-ylmethyl)ureido)phenyl]nicotinamide

16 ml of saturated NaHCO₃ solution, followed by 0.173 g (0.15 mm) of Pd(PPh₃)₄), are added, at ambient temperature under an argon atmosphere, to a solution of 0.320 g (1.5 mm) of the compound obtained in stage 1.2 and 0.648 g (1.65 mm) of the compound obtained in stage 1.3 in 40 ml of dimethoxyethane and 8 ml of ethanol. The reaction medium is immersed in an oil bath preheated to 100° C. and heating is carried out at this temperature for 3 h. The solvents are evaporated under reduced pressure and the residue is taken up in a dichloromethane (DCM)/water mixture. The precipitate is filtered off. The filtrate is subsequently purified by chromatography on a silica column (DCM:MeOH-10:0.7). After evaporating the solvents, the residue is taken up in ethyl acetate and then filtered. The filtrate is then dried under vacuum at 60° C. 0.387 g of a solid is obtained. The yield is thus 63.7%. M.p.=260-263° C. (LC/MS; MH+ 405, tᵣ=5.61 min). ¹H NMR (d₆-DMSO, 250 MHz): 1.21 (t, 3), 2.75 (d, 3), 3.52 (qd, 2), 4.35 (d, 2), 6.80 (t, 1), 7.09 (d, 1), 7.38 (dd, 1), 7.52 (d, 2), 7.74 (td, 1), 7.93 (d, 1), 8.02 (d, 2), 8.41 (m, 1), 8.47 (m, 1), 8.48 (m, 1), 8.55 (d, 1), 8.88 (s, 1).

### Example 2

### 2-Amino-N-methyl-6-[4-(3-(pyridin-3-ylmethyl)ureido)phenyl]-nicotinamide (Compound No. 3)

### 2.1: 2-Amino-6-chloronicotinic acid

9.6 g (50 mm) of 2,6-dichloronicotinic acid are mixed in a glass autoclave with 60 ml of 32% aqueous ammonia solution. The reaction medium is immersed in an oil bath preheated to 130° C. and heating is carried out at this temperature for 68 h. The solution is allowed to return to ambient temperature. The reaction medium is concentrated under reduced pressure. The residue is taken up in 200 ml of water and ice and acidified to pH 2 with a concentrated HCl of solution. Ethyl acetate is added and the medium is then stirred for 5 minutes and filtered. The aqueous phase is separated by settling and the organic phase is washed with a saturated in NaCl solution. The organic phase is dried over sodium sulphate and filtered, and the solvent is evaporated. 5.83 g of product (Yd: 67.5%) are obtained (LC/MS; MH+ 173, tᵣ=6.03 min).

### 2.2: 2-Amino-6-chloro-N-methylnicotinamide

6.26 ml (45 mm) of triethylamine, 15 ml (30 mm) of a 2N solution of methylamine in THF and 6.17 g (14 mm) of BOP are successively added to a solution of 2.59 g (15 mm) of the compound obtained in stage 1.1 in 50 ml of anhydrous THF. The medium is stirred at ambient temperature for 18 h, followed by evaporation of the solvent under reduced pressure. The residue is taken up in ethyl acetate and then washed successively with water, a 3% solution of KHSO₄ in water, a 10% solution of Na₂CO₃ in water and a saturated NaCl solution. 2.046 g of nicotinamide are obtained. The yield is quantitative. M.p.=204-207° C. (LC/MS; MH+ 186, tᵣ=6.72 min).

### 2.3: 2-Amino-N-methyl-6-[4-(3-(pyridin-3-ylmethyl)ureido)phenyl]nicotinamide

16 ml of saturated NaHCO₃ solution, followed by 0.231 g (0.20 mm) of Pd(PPH₃)₄ are added, at ambient temperature under an argon atmosphere, to a solution of 0.317 g (2 mm) of the compound obtained in stage 2.2 and 0.777 g (2.20 mm) of the compound obtained in stage 1.3 in 40 ml of dimethoxyethane and 8 ml of ethanol. The reaction medium is immersed in an oil bath and heated at 100° C. for 3 h. The solvents are evaporated under reduced pressure. The residue is taken up in a DCM/water mixture. The precipitate is filtered off and then purified by chromatography on a silica column (dichloromethane (DCM):MeOH-10:1). 0.507 g of nicotinamide derivative is obtained. The yield is thus 67.3%. M.p.=234-236° C. (LC/MS; MH+ 376, tᵣ=4.47 min). ¹H NMR (d₆-DMSO, 400 MHz): 2.75 (d, 3), 4.33 (d, 2), 6.79 (t, 1), 7.10 (d, 1), 7.15 (bs, 2), 7.36 (dd, 1), 7.49 (d, 2), 7.72 (td, 1), 7.91 (d, 1), 7.95 (d, 2), 8.34 (q, 1), 8.46 (d, 1), 8.53 (bs, 1), 8.84 (s, 1).

### Example 3

### 2-(2-(Dimethylamino)ethylamino)-N-methyl-6-[4-(3-(pyridin-3-ylmethyl)-ureido)phenyl]nicotinamide (Compound No. 7)

### 3.1: 6-Chloro-2-(2-(dimethylamino)ethylamino)nicotinic acid hydrochloride

24.0 g (0.125 m) of 2,6-dichloronicotinic acid are mixed in a round-bottomed flask with 124.53 ml of N,N-dimethylaminoethylamine. The solution is then stirred at ambient temperature for 6 days. The excess amine is subsequently evaporated under reduced pressure. The residue is taken up in the minimum amount of water. The reaction medium is cooled with an ice bath and acidified to pH 3 with a 5N HCl solution. Finally, the precipitate is filtered off and washed with cold water in order to be finally dried under vacuum over P₂O₅ at 60° C. 26 g (yd=87.7%) of white solid are obtained. M.p.=170-172° C. (LC/MS, MH+ 244, tᵣ=4.73 min).

### 3.2: 6-Chloro-2-(2-(dimethylamino)ethylamino)-N-methylnicotinamide

0.62 ml (4.9 mm) of triethylamine, 1.64 ml (3.3 mm) of a 2N solution of methylamine in THF and 0.68 g (1.52 mm) of BOP are successively added to a solution of 0.400 g (1.6 mm) of the compound obtained in stage 3.1 in 20 ml of THF and two drops of DMF. The medium is stirred at ambient temperature overnight, followed by evaporation of the solvent under reduced pressure. The residue is taken up in ethyl acetate and then successively washed with water, a 3% solution of KHSO₄ in water, a 10% solution of Na₂CO₃ in water and a saturated NaCl solution. 0.3 g (yd=71%) of nicotinamide derivative is obtained. (LC/MS; MH+ 257, tᵣ=4.24 min).

### 3.3: 2-(2-(Dimethylamino)ethylamino)-N-methyl-6-[4-(3-(pyridin-3-ylmethyl)ureido)-phenyl]nicotinamide

20 ml of saturated NaHCO₃ solution, followed by 0.135 g (0.12 mm) of Pd(PPh₃)₄, are added, at ambient temperature under an argon atmosphere, to a solution in a three-necked flask of 0.300 g (1.2 mm) of the compound obtained in stage 3.2 and 0.454 g (1.29 mm) of the compound obtained in stage 1.3 in 40 ml of dimethoxyethane and 8 ml of ethanol. The mixture is heated at 100° C. for 3 h. The solvents are evaporated under reduced pressure and the residue is taken up in water. The precipitate is filtered off and then purified by flash chromatography (DCM; MeOH 10-30%; NH₄OH 1%). 0.070 g of solid is obtained. The yield is thus 13.8%. M.p.=163-165° C. (LC/MS; MH+ 448, tᵣ=4.53 min). ¹H NMR (d₆-DMSO, 250 MHz): 2.22 (s, 6), 2.50 (m, 2), 2.75 (d, 3), 3.59 (q, 2), 4.34 (d, 2), 6.82 (t, 1), 7.08 (d, 1), 7.37 (dd, 1), 7.51 (d, 2), 7.73 (d, 1), 7.92 (d, 1), 8.01 (d, 2), 8.36 (q, 1), 8.46 (dd, 1), 8.54 (s, 1), 8.58 (t, 1), 8.88 (s, 1).

### Example 4

### N-(2-(Diisopropylamino)ethyl)-2-ethylamino-6-[4-(3-(pyridin-3-ylmethyl)ureido)phenyl]nicotinamide (Compound No. 8)

### 4.1: 2-Ethylamino-6-[4-(3-(pyridin-3-ylmethyl)ureido)phenyl]nicotinic acid

50 ml of saturated NaHCO₃ solution, followed by 1.152 g (1.00 mm) of Pd(PPh₃)₄, are added, at ambient temperature under an argon atmosphere, to a solution in a three-necked flask of 2.0 g (9.97 mm) of the compound obtained in stage 1.1 and 3.873 g (10.97 mm) of the compound obtained in stage 1.3 in 200 ml of dimethoxyethane and 40 ml of ethanol. The mixture is heated at 90° C. for 20 h. The solvents are evaporated under reduced pressure and the residue is taken up in an ethyl acetate/water mixture. The aqueous phase is separated by settling and then acidified to pH=6 with a concentrated HCl solution. The precipitate is filtered off, washed with water and dried in an oven. 3.8 g of solid are obtained. Yd: 97.4%. M.p.=216-218° C. (LC/MS; MH+ 392, tᵣ=5.20 min).

### 4.2: N-(2-(Diisopropylamino)ethyl)-2-ethylamino-6-[4-(3-(pyridin-3-ylmethyl)ureido)-phenyl]nicotinamide

0.27 ml (1.92 mm) of triethylamine, 0.22 ml (1.28 mm) of 2-diisopropylaminoethylamine and 0.263 g (0.60 mm) of BOP are successively added to a solution of 0.250 g (0.64 mm) of the compound obtained in stage 4.1 in 20 ml of THF. The reaction medium is stirred at ambient temperature for 3 days, followed by evaporation of the solvent under reduced pressure. The residue is taken up in DCM and then successively washed with water and a saturated NaCl solution. The organic phase is finally dried and concentrated. The residue is purified by flash chromatography (DCM; MeOH 5-30%; NH₄OH 1%). 0.25 g (yd=75.5%) of white solid is obtained. M.p.=160-162° C. (LC/MS; MH+ 518, tᵣ=5.32 min). ¹H NMR (d₆-DMSO 250 MHz): 0.98 (d, 12), 1.21 (t, 3), 2.52 (m, 2), 2.97 (m, 2), 3.17 (m, 2), 3.52 (m, 2), 4.34 (d, 2), 6.79 (t, 1), 7.08 (d, 1), 7.36 (dd, 1), 7.50 (d, 2), 7.72 (td, 1), 7.92 (d, 1), 8.00 (d, 2), 8.33 (t, 1), 8.46 (m, 2), 8.54 (s, 1), 8.86 (s, 1).

### Example 5

### N-Methyl-2-[(pyridin-4-ylmethyl)amino]-6-[4-(3-(pyridin-3-ylmethyl)-ureido)phenyl]nicotinamide (Compound No. 15)

### 5.1: 6-Chloro-2-[(pyridin-4-ylmethyl)amino]nicotinic acid

A solution of 1.2 g (6.25 mm) of 2,6-dichloronicotinic acid and of 1.91 ml (18.75 mm) of 4-pyridylmethylamine in 10 ml of isopropanol is heated in a glass autoclave at 90° C. for 12 h. The precipitate is filtered off and washed with ethyl acetate. The solvent is evaporated under reduced pressure. The residue is taken up in 2 ml of water. The reaction medium is acidified using acetic acid until precipitation has occurred. The precipitate is filtered off and then washed with cold water in order to be finally dried in an oven over P₂O₅. 1.1 g (yd=66.7%) of white solid are obtained. M.p.=217-220° C. (LC/MS; MH+ 264, tᵣ=4.99 min).

### 5.2: 6-Chloro-N-methyl-2-[(pyridin-4-ylmethyl)amino]nicotinamide

0.47 ml (4.6 mm) of triethylamine, 1.52 ml (3.0 mm) of a 2N solution of methylamine in THF and 0.497 g (1.12 mm) of BOP are successively added to a solution of 0.400 g (1.5 mm) of the compound obtained in stage 5.1 in 20 ml of THF. The medium is stirred at ambient temperature for 18 h, followed by evaporation of the solvent under reduced pressure. The residue is taken up in DCM and then successively washed with water, a 3% solution of KHSO₄ in water, a 10% solution of Na₂CO₃ in water and a saturated NaCl solution. The organic phase is dried and the DCM is evaporated. The residue is purified by flash chromatography (DCM; MeOH 1-5%). 0.3 g of nicotinamide (yd=71.4%) is obtained (LC/MS; MH+ 277, tᵣ=5.04 min).

### 5.3: N-Methyl-2-[(pyridin-4-ylmethyl)amino]-6-[4-(3-(pyridin-3-ylmethyl)ureido)phenyl]-nicotinamide

20 ml of a saturated NaHCO₃ solution, followed by 0.173 g (0.15 mm) of Pd(PPh₃)₄, are added, at ambient temperature under an argon atmosphere, to a solution of 0.300 g (1.1 mm) of the compound obtained in stage 5.2 and 0.421 g (1.19 mm) of the compound obtained in stage 1.3 in 40 ml of dimethoxyethane and 8 ml of ethanol. The mixture is heated at 100° C. for 6 h. The solvents are evaporated under reduced pressure and the residue is taken up in a DCM/water mixture. The precipitate is filtered off. The organic phase, after extraction, is concentrated. The precipitate and the residue are subsequently purified by flash chromatography (DCM; MeOH 1-15%). 0.4 g of solid is obtained. The yield is thus 80%. M.p.=218-219° C. (LC/MS; MH+ 468, tᵣ=4.96 min). ¹H NMR (d₆-DMSO, 400 MHz): 2.78 (s, 3), 4.33 (m, 2), 4.75 (m, 2), 6.78 (q, 1), 7.13 (m, 1), 7.35 (m, 3), 7.44 (m, 2), 7.71 (m, 1), 7.84 (m, 2), 7.97 (m, 1), 8.49 (m, 4), 8.53 (m, 1), 8.80 (m, 1), 9.03 (m, 1).

### Example 6

### 6-{-4-[3-(6-Aminopyridin-3-ylmethyl)ureido]phenyl}-2-ethylamino-N-methylnicotinamide (Compound No. 21)

### 6.1: 6-(4-Aminophenyl)-2-ethylamino-N-methylnicotinamide

20 ml of saturated NaHCO₃ solution, followed by 0.325 g (0.28 mm) of Pd(PPh₃)₄, are added, at ambient temperature under an argon atmosphere, to a solution of 0.600 g (2.81 mm) of the compound obtained in stage 1.2 and 0.677 g (3.1 mm) of 4-(4,4,5,5-tetramethyl-1,3,2-dioxaborolan-2-yl)phenylamine in 40 ml of dimethoxyethane and 8 ml of ethanol. The mixture is heated at 90° C. for 3 h. The solvents are evaporated under reduced pressure and the residue is taken up in a DCM/water mixture. The precipitate is filtered off. The organic phase, after washing with water and a saturated NaCl solution, is dried and concentrated. The filtrate and the residue are subsequently purified by flash chromatography (DCM; MeOH 0-1%). 0.680 g of white solid is obtained. The yield is thus 89.5%. (LC/MS; MH+ 271, tᵣ=6.01 min).

### 6.2: 6-{-4-[3-(6-Aminopyridin-3-ylmethyl)ureido]phenyl}-2-ethylamino-N-methylnicotinamide

0.369 g (3.02 mm) of dimethylaminopyridine and 0.773 g (3.02 mm) of disuccinimidyl carbonate are added, at ambient temperature under an argon atmosphere, to a solution of 0.680 g (2.52 mm) of the compound obtained in stage 6.1 in 80 ml of anhydrous THF and the mixture is then stirred for 12 h. After the addition of 2.10 ml (15.09 mm) of triethylamine and 0.482 g (3.02 mm) of 5-aminomethylpyridin-2-ylamine, the mixture is stirred at ambient temperature for 18 h. The reaction medium is subsequently concentrated. The residue is taken up in water and DCM and then filtered. The insoluble material is again washed with water and DCM in order to be finally dried in an oven. The product is purified by flash chromatography (DCM; MeOH 1-10%). 0.45 g (yd 42.6%) of product is obtained. M.p.=223-226° C. (LC/MS; MH+ 420, tᵣ=5.26 min). ¹H NMR (d₆-DMSO, 250 MHz): 1.16 (t, 3), 2.67 (d, 3), 3.47 (m, 2), 4.06 (d, 2), 5.80 (bs, 2), 6.38 (d, 1), 6.47 (t, 1), 7.03 (d, 1), 7.31 (dd, 1), 7.45 (d, 2), 7.82 (d, 1), 7.88 (d, 1), 7.96 (d, 2), 8.34 (q, 1), 8.42 (t, 1), 8.66 (s, 1).

### Example 7

### N-Methyl-2-phenylamino-6-[4-(3-(pyridin-3-ylmethyl)ureido)phenyl]-nicotinamide (Compound No. 22)

### 7.1: 6-Chloro-2-(phenylamino)nicotinic acid

1 ml (10.9 mm) of aniline is dissolved in 15 ml of anhydrous THF in a three-necked flask under argon and 16.7 ml (16.7 mm) of lithium bis(trimethylsilyl)amide (1M in THF) are added dropwise at a temperature of −75° C. This medium is stirred at this temperature for 1 h. 1 g (5.2 mm) of 2,6-dichloronicotinic acid dissolved in 10 ml of anhydrous THF is added to the reaction medium. The medium is allowed to return to ambient temperature and stirred at this temperature for 12 h. 2-3 ml of water are added to the reaction medium. It is then cooled in an ice bath and acidified to pH 2 with a 5N HCl solution. Extraction is carried out with ethyl acetate. The aqueous phase is subsequently extracted several times with ethyl acetate. The organic phases are subsequently washed with water and saturated NaCl solution. The organic phase is dried and then concentrated. (The residue is purified by flash chromatography). 1.1 g (85.3%) of white solid are obtained. M.p.=181-185° C. (LC/MS; MH+ 249, tᵣ=6.99 min).

### 7.2: 6-Chloro-N-methyl-2-(phenylamino)nicotinamide

0.84 ml (6 mm) of triethylamine, 2.01 ml (4.0 mm) of a 2N solution of methylamine in THF and 0.658 g (1.5 mm) of BOP are successively added to a solution of 0.500 g (2.01 mm) of the compound obtained in stage 7.1 in 20 ml of THF. The medium is stirred at ambient temperature for 18 h, followed by evaporation of the solvent under reduced pressure. The residue is taken up in DCM and then successively washed with water and a saturated NaCl solution. The organic phase is dried and then concentrated. The residue is purified by flash chromatography (DCM:Heptane-1:1). 0.35 g of nicotinamide is obtained. (Yd=66.5%). (LC/MS; MH+ 262, tᵣ=9.49 min).

### 7.3: N-Methyl-2-phenylamino-6-[4-(3-(pyridin-3-ylmethyl)ureido)phenyl]nicotinamide

15 ml of saturated NaHCO₃ solution, followed by 0.155 g (0.13 mm) of Pd(PPh₃)₄, are added, at ambient temperature under an argon atmosphere, to a solution of 0.350 g (1.3 mm) of the compound obtained in stage 7.2 and 0.520 g (1.5 mm) of the compound obtained in stage 1.3 in 40 ml of dimethoxyethane and 8 ml of ethanol. The mixture is heated at 90° C. for 4 h. The solvents are evaporated under reduced pressure and the residue is taken up in a DCM/water mixture. The precipitate is filtered off. The organic phase, after washing with water and a saturated NaCl solution, is dried and concentrated. The precipitate and the residue are subsequently purified by flash chromatography (DCM; MeOH 1-10%). 0.530 g of white solid is obtained. The yield is thus 87.6%. M.p.=234-236° C. (LC/MS; MH+ 453, tᵣ=6.70 min). ¹H NMR (d₆-DMSO, 250 MHz): 2.77 (d, 3), 4.30 (d, 2), 6.79 (t, 1), 6.94 (t, 1), 7.27-7.38 (unresolved peak, 4), 7.52 (d, 2), 7.69 (td, 1), 7.74 (d, 2), 7.99 (d, 2), 8.09 (d, 1), 8.43 (d, 1), 8.51 (d, 1), 8.67 (q, 1), 8.85 (s, 1), 11.15 (s, 1).

### Example 8

### [4-(6-Ethylamino-5-(methylcarbamoyl)pyridin-2-yl)phenyl]carbamic acid pyridin-3-ylmethyl ester (compound No. 29)

### 8.1: Pyridin-3-ylmethyl [4-(4,4,5,5-tetramethyl-1,3,2-dioxaborolan-2-yl)phenyl]carbamate

5.72 ml (41.08 mm) of triethylamine are introduced dropwise into a mixture of 1.5 g (6.85 mm) of 4-(4,4,5,5-tetramethyl-1,3,2-dioxaborolan-2-yl)phenylamine and of 1.219 g (4.11 mm) of triphosgene in 200 ml of THF, cooled with an ice/water bath to a temperature of between 0° C. and 5° C. After stirring at a temperature of between 0° C. and 5° C. for 1 h, 0.837 g (7.67 mm) of 3-pyridylcarbinol is added to the reaction medium. The reaction medium is stirred for 20 h while allowing the temperature to rise to ambient temperature. The THF is evaporated. The residue is taken up in water and then extracted with ethyl acetate. The organic phase is washed with H₂O and then with an H₂O/NaCl solution in order to be subsequently dried over Na₂SO₄, filtered and evaporated. The residue is subsequently purified by flash chromatography (DCM; MeOH 1-5%). 2.0 g (yd=82.5%) of white solid composed of 76% of the expected compound and 24% of the corresponding boronic acid are obtained (LC/MS; MH+ 355 and 273, tᵣ=8.62 and 5.78 min).

### 8.2: [4-(6-Ethylamino-5-(methylcarbamoyl)pyridin-2-yl)phenyl]carbamic acid pyridin-3-ylmethyl ester

15 ml of saturated NaHCO₃ solution, followed by 0.135 g (0.12 mm) of Pd(PPh₃)₄, are added, at ambient temperature under an argon atmosphere, to a solution of 0.250 g (1.17 mm) of the compound obtained in stage 1.2 and 0.456 g (1.29 mm) of the compound obtained in stage 8.1 in 38 ml of dimethoxyethane and 7 ml of ethanol. The reaction medium is immersed in an oil bath preheated to 90° C. and heating is carried out at this temperature for 3 h. The solvents are evaporated under reduced pressure and the residue is taken up in a DCM/H₂O mixture. The precipitate is filtered off. The filtrate is subsequently purified by flash chromatography on a silica column (DCM; MeOH 5-10%). After evaporating the solvents, the residue is taken up in ethyl acetate and then filtered. The filtrate is then dried under vacuum at 60° C. 0.230 g of solid is obtained. The yield is thus 48.5%. M.p.=234-235° C. (LC/MS; MH+ 406, tᵣ=6.74 min).

### Example 9

### 2-Ethylamino-N-(2-(piperidin-1-yl)ethyl)-6-[4-(3-(pyridin-3-ylmethyl)-ureido)phenyl]nicotinamide (compound No. 13)

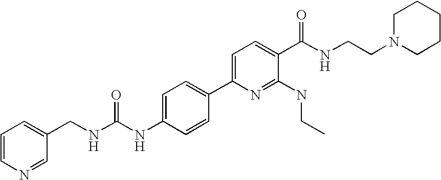

0.27 ml (1.92 mm) of triethylamine, 0.18 ml (1.28 mm) of 2-(piperidin-1-yl)ethylamine and 0.263 g (0.60 mm) of BOP are successively added to a solution of 0.25 g (0.64 mm) of the compound obtained in stage 4.1 in 20 ml of THF. The mixture is stirred at ambient temperature for 18 h. The medium is concentrated and then the residue is taken up in water. Extraction is carried out with DCM and washing is carried out successively with water and then a saturated sodium chloride solution. The organic phase is dried on sodium sulphate, filtered and evaporated. The residue is purified by flash chromatography (DCM; MeOH 1-20%). 0.23 g (yd=71.9%) is obtained. M.p.=164-165° C. LC/MS; MH+ 502, tᵣ=5.31 min. ¹H NMR (d₆-DMSO, 250 MHz): 1.21 (t, 3), 1.29-1.56 (unresolved peak, 6), 2.33-2.48 (unresolved peak, 6), 3.30 (m, 2), 3.52 (m, 2), 4.36 (d, 2), 6.79 (t, 1), 7.09 (d, 1), 7.37 (t, 1), 7.51 (d, 2), 7.73 (d, 1), 7.92 (d, 1), 8.00 (d, 2), 8.33 (t, 1), 8.41 (t, 1), 8.46 (d, 1), 8.54 (s, 1), 8.86 (s, 1).

### Example 10

### 2-Ethoxy-N-methyl-6-[4-(3-(pyridin-3-ylmethyl)ureido)phenyl]-nicotinamide (Compound No. 63)

### 10.1: 2,6-Dichloro-N-methylnicotinamide

1.0 g (5.2 mmol) of 2,6-dichloronicotinic acid is dissolved in 10 ml of anhydrous THF in a 25 ml round-bottomed flask under a nitrogen atmosphere. 930 mg (5.7 mmol) of N,N′-carbonyldiimidazole are added and the mixture is stirred at ambient temperature for 30 min. 2.8 ml (5.7 mmol) of a 2.0M solution of methylamine in THF are added and the mixture is stirred at ambient temperature for 4 h. The mixture is hydrolysed with a saturated aqueous NH₄Cl solution (10 ml) and extracted with ethyl acetate (4×10 ml). The organic phases are combined and then washed with 10 ml of a saturated aqueous NaCl solution. After separation, the organic phase is dried over MgSO₄ and filtered, and the solvent is evaporated under reduced pressure. The residue is purified by flash chromatography on a silica column (40-63 μm) (eluent: AcOEt). The pure fractions are collected and then the solvent is evaporated under reduced pressure in order to obtain 380 mg (1.8 mmol) of the compound in the form of a white powder. Yd: 36%. ¹H NMR, CDCl₃, 300 MHz: 2.98 (d, J=4.9 Hz, 3H), 6.77 (bs, 1H), 7.30 (d, J=8.0 Hz, 1H), 7.95 (d, J=8.0 Hz, 1H).

### 10.2: 2-Chloro-6-ethoxy-N-methylnicotinamide

380 mg (1.8 mmol) of compound 10.1 are dissolved in 10 ml of absolute ethanol in a 25 ml round-bottomed flask under a nitrogen atmosphere. 47 mg (2.0 mmol) of sodium are added and then the mixture is stirred at 70° C. for 16 h. The solvent is evaporated under reduced pressure and the residue is taken up in 25 ml of DCM. The precipitate is filtered off, triturated in ethyl ether and dried. 300 mg (1.4 mmol) of compound are isolated in the form of a white solid. Yd: 74%. ¹H NMR, CDCl₃ (300 MHz): 1.40 (t, J=7.1 Hz, 3H), 2.92 (d, J=6.7 Hz, 3H), 4.47 (q, J=7.1 Hz, 2H), 6.95 (d, J=8.0 Hz, 1H), 7.73 (bs, 1H), 8.36 (d, J=8.0 Hz, 1H).

### 10.3: 6-(4-Aminophenyl)-2-ethoxy-N-methylnicotinamide

300 mg (1.4 mmol) of compound 10.2 are dissolved in a mixture of 40 ml of DME and 10 ml of ethanol in a 100 ml round-bottomed flask. 340 mg (1.5 mmol) of p-aniline boronic ester are added, followed by 15 ml of a saturated aqueous NaHCO₃ solution. The mixture is degassed using a stream of nitrogen, then 162 mg (0.1 mmol) of Pd(PPh₃)₄ are added and the mixture is heated at reflux for 16 h. After returning to ambient temperature, the mixture is filtered through a filter paper and the solvents are evaporated under reduced pressure. The residue is taken up in 25 ml of water and then extracted with 3×25 ml of AcOEt. The organic phases are combined and then washed with 25 ml of a saturated aqueous NaCl solution. After separation, the organic phase is dried over MgSO₄ and filtered, and the solvent is evaporated under reduced pressure. The residue is purified by flash chromatography on a silica column (40-63 μm) (eluent: EtOAc). The pure fractions are collected and then the solvent is evaporated under reduced pressure in order to obtain 380 mg (1.4 mmol) of compound in the form of a pale yellow powder. Yd: quantitative. ¹H NMR, CDCl₃ (300 MHz): 1.51 (t, J=7.1 Hz, 3H), 3.02 (d, J=4.8 Hz, 3H), 3.90 (bs, 2H), 4.67 (q, J=7.1 Hz, 2H), 6.73 (d, J=8.7 Hz, 2H), 7.37 (d, J=8.0 Hz, 1H), 7.90 (d, J=8.7 Hz, 2H), 8.01 (bs, 1H), 8.49 (d, J=8.0 Hz, 1H).

### 10.4: 2-Ethoxy-N-methyl-6-[4-(3-(pyridin-3-ylmethyl)ureido)phenyl]nicotinamide

380 mg (1.4 mmol) of compound 10.3 are dissolved in 50 ml of anhydrous THF in a 100 ml round-bottomed flask under a nitrogen atmosphere. 540 mg (2.1 mmol) of N,N′-disuccinimidyl carbonate and 256 mg (2.1 mmol) of dimethylaminopyridine are added and then the mixture is stirred at ambient temperature for 16 h. 585 μl (4.2 mmol) of triethylamine and a solution of 230 mg (2.1 mmol) of pyridin-3-ylmethylamine dissolved in 10 ml of anhydrous THF are added and then the mixture is stirred at ambient temperature for 8 h. The solvent is evaporated under reduced pressure. The residue is purified by flash chromatography on a silica column (40-63 μm) (eluent: DCM/MeOH, 90/10). The pure fractions are collected and then the solvent is evaporated under reduced pressure in order to obtain 20 mg (0.05 mmol) of the desired compound in the form of a white powder. Yd: 3%; M.p.=200° C. ¹H NMR, CDCl₃ (300 MHz): 1.44 (t, J=7.0 Hz, 3H), 2.84 (d, J=4.7 Hz, 3H), 4.34 (d, J=5.8 Hz, 2H), 4.60 (q, J=7.0 Hz, 2H), 6.81 (t, J=5.8 Hz, 1H), 7.37 (m, 1H), 7.54 (d, J=8.8 Hz, 2H), 7.59 (d, J=8.0 Hz, 1H), 7.72 (d, J=7.8 Hz, 1H), 8.03 (d, J=8.8 Hz, 2H), 8.12 (m, 1H), 8.20 (d, J=8.0 Hz, 1H), 8.46 (m, 1H), 8.54 (s, 1H), 8.91 (s, 1H).

### Example 11

### 4-Ethylamino-2-[4-(3-(pyridin-3-ylmethyl)ureido)phenyl]pyrimidine-5-carboxylic acid (2-(piperidin-1-yl)ethyl)amide (Compound No. 80)

### 11.1: 2,4-Dichloropyrimidine-5-carbonyl chloride

2,4-Dihydroxypyrimidine-5-carboxylic acid (10 g, 64 mmol) is dispersed in POCl₃ (45 ml) at 0° C. PCl₅ (46.6 g, 224 mmol) is carefully added and the mixture is stirred under gentle reflux for 16 h. The slightly yellow solution is evaporated under reduced pressure and the solid is washed with toluene, and the solution is filtered and the filtrate evaporated to give 13.4 g (yd: 99%) of the compound. ¹H NMR, d₆-DMSO (300 MHz): 9.13 (s, 1H).

### 11.2: 2,4-Dichloropyrimidine-5-carboxylic acid ethyl ester

Compound 11.1 (13.5 g, 64 mmol) is dissolved in THF (100 ml). Ethanol (15 ml) is added and the mixture is stirred at ambient temperature for 10 min. The solvents are evaporated and an oil is recovered and hydrolysed with a saturated K₂CO₃ solution and extracted with AcOEt (3×250 ml). The organic phase is washed with an NaCl solution (100 ml) and dried over Na₂SO₄. After filtering and evaporating, an orange oil is recovered (14 g, yd: 99%). ¹H NMR, d₆-DMSO (300 MHz): 9.16 (s, 1H), 4.37 (q, 2H, J=7.11 Hz), 1.34 (t, 3H, J=7.11 Hz).

### 11.3: 2-Chloro-4-(ethylamino)pyrimidine-5-carboxylic acid ethyl ester

Compound 11.2 (14 g, 63.3 mmol) is dissolved in 150 ml of THF. Triethylamine (13 ml, 94.95 mmol) and a solution of ethylamine in THF (32 ml, 63.3 mmol) are added. The mixture is stirred at ambient temperature for 16 h. It is filtered and the solvent is evaporated. The residue is purified by column chromatography (40-63 μm, eluent: AcOEt/cyclohexane:20/80). The fractions are recovered and the solvent is evaporated. A white solid is obtained (9.2 g, yd: 63%). ¹H NMR d₆-DMSO (300 MHz): 8.59 (s, 1H), 8.50 (bs, 1H), 4.30 (q, 2H, J=7.08 Hz), 3.47 (m, 2H, J=7.08 Hz), 1.15 (t, 3H, J=7.17 Hz).

### 11.4: 2-Chloro-4-(ethylamino)pyrimidinecarboxylic acid

Compound 11.3 (9.2 g, 40 mmol) is dissolved in THF (250 mg). Water and then LiOH.H₂O 2.5 g, 60 mmol) are added and the mixture is left stirring at ambient temperature for 16 h. The solvent is evaporated and a 1N HCl solution is added until precipitation is complete. After filtration, the solid is dried at 60° C. overnight. 8.0 g (yd: 99%) of the compound are obtained in the form of a white solid. ¹H NMR, d₆-DMSO (300 MHz): 8.65 (bs, 1H), 8.55 (s, 1H), 3.45 (m, 2H), 1.15 (t, 3H, J=7.17 Hz).

### 11.5: 4-Ethylamino-2-[4-(3-pyridin-3-ylmethyl)ureido)phenyl]-pyrimidine-5-carboxylic acid

1.613 g (8 mm) of the compound obtained in stage 11.4, 3.11 g (8.8 mm) of the compound obtained in stage 8.1, 160 ml of DME, 32 ml of ethanol and 40 ml of saturated NaHCO₃ solution are placed in a three-necked flask under an argon atmosphere. The mixture is degassed for 30 min and then 0.925 g (0.8 mm) of Pd(PPh₃)₄ is added. The mixture is heated at 100° C. for 6 h. The solvents are evaporated and the residue is taken up in water. The pH is adjusted to 3-4 with a 1N HCl solution. The precipitate is filtered off and dried under vacuum over P₂O₅. The precipitate is taken up in 400 ml of methanol at reflux and allowed to cool. The product is filtered off and dried under vacuum. 859 mg are obtained and are used as is in the following stage (LC/MS; MH+ 393, tᵣ=4.90 min).

### 11.6: 4-Ethylamino-2-[4-(3-(pyridin-3-ylmethyl)ureido)phenyl]pyrimidine-5-carboxylic acid (2-(piperidin-1-yl)ethyl)amide

0.44 g (1.12 mm) of the compound obtained in stage 11.5 are placed in 30 ml of THF in a round-bottomed flask. 0.47 ml (3.36 mm) of triethylamine, 0.32 ml (2.24 mm) of 2-(piperidin-1-yl)ethylamine and 0.496 g (1.12 mm) of BOP are added. The mixture is stirred at ambient temperature for 18 h. The solvents are evaporated and the residue is taken up in ethyl acetate. The organic phase is washed with water and then a saturated NaCl solution. It is dried over Na₂SO₄, filtered and evaporated. The residue is purified by flash chromatography (DCM:MeOH 99:1 to 80:20). 220 mg are obtained. Yd: 33.6% (LC/MS; MH+ 503, tᵣ=4.71 min). ¹H NMR (250 MHz, d₆-DMSO) δ ppm: 1.21 (t, 3), 1.44 (m, 2), 1.60 (m, 4), 2.70 (m, 6), 3.46 (m, 2), 3.58 (quint, 2), 4.35 (d, 2), 6.95 (t, 1), 7.38 (dd, 1), 7.54 (d, 2), 7.74 (dt, 1), 8.27 (d, 2), 8.47 (m, 1), 8.55 (d, 1), 8.72 (m, 3), 9.11 (s, 1).

### Example 12

### 6-{-4-[3-(6-(Aminopyridin)-3-ylmethyl)ureido]phenyl}-2-ethylamino-N-(2-(piperidin-1-yl)ethyl)nicotinamide (Compound No. 81)

### 12.1: 6-Chloro-2-ethylamino-N-(2-(piperidin-1-yl)ethyl)nicotinamide

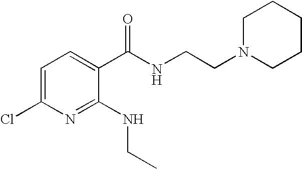

5.0 g (24.92 mm) of 6-chloro-2-(ethylamino)nicotinic acid (Ex. 1.1) are dissolved in 300 ml of THF in a round-bottomed flask. 10.41 ml (74.77 mm) of triethylamine, then 7.08 ml (49.84 mm) of 1-(2-aminoethyl)piperidine and subsequently 11.02 g (24.92 mm) of BOP are added. The mixture is stirred at ambient temperature for 15 h. The solvent is evaporated and the residue is taken up in ethyl acetate. The organic phase is washed with water and then a saturated NaCl solution. It is dried over Na₂SO₄, filtered and evaporated. The residue is purified by flash chromatography (gradient CH₂Cl₂-MeOH 1 to 10%). 7.5 g (yd: 96.8%) are obtained (LC/MS; MH+ 311, tᵣ=1.01 min).

### 12.2: 6-(4-Aminophenyl)-2-ethylamino-N-(2-(piperidin-1-yl)ethyl)nicotinamide

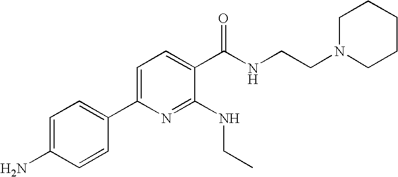

6.0 g (19.3 mm) of the compound obtained in stage 12.1, 4.65 g (21.23 mm) of 4-(4,4,5,5-tetramethyl-1,3,2-dioxaborolan-2-yl)aniline, 400 ml of DME, 60 ml of ethanol and 250 ml of a saturated NaHCO₃ solution are placed in a three-necked flask under an argon atmosphere. The mixture is degassed for 30 min and then 2.23 g (1.93 mm) of Pd(PPh₃)₄ are added. The mixture is brought to reflux for 10 h. The solvents are evaporated and the residue is taken up in CH₂Cl₂. The organic phase is washed with water and then a saturated NaCl solution. The organic phase is dried over Na₂SO₄, filtered and evaporated. The residue is purified by flash chromatography (gradient CH₂Cl₂-MeOH 1 to 15%). 6.4 g (yd: 90.2%) are obtained (LC/MS; MH+ 368, tᵣ=0.65 min).

### 12.3: [5-(3-{4-[6-Ethylamino-5-(2-(piperidin-1-yl)ethylcarbamoyl)pyridin-2-yl]phenyl}-ureidomethyl)pyridin-2-yl]carbamic acid tert-butyl ester

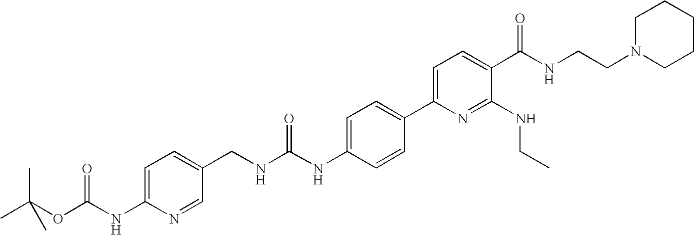

0.8 g (2.18 mm) of the compound obtained in stage 12.2 are placed in 80 ml of THF in a round-bottomed flask. 0.67 g (2.61 mm) of DSC and 0.319 g (2.61 mm) of DMAP are added. The mixture is stirred at ambient temperature for 18 h. 0.91 ml (6.53 mm) of triethylamine and 0.583 g (2.61 mm) of (5-(aminomethyl)pyridin-2-yl)carbamic acid tert-butyl ester are subsequently added and the mixture is stirred at ambient temperature for 15 h. The solvents are evaporated and filtration is carried out. Purification is carried out by flash chromatography (gradient CH₂Cl₂-MeOH 1 to 20%). 1 g (yd: 74.5%) is obtained. (LC/MS; MH+ 617, tᵣ=6.6 min).

### 12.4: 6-{-4-[3-(6-(Aminopyridin)-3-ylmethyl)ureido]phenyl}-2-ethylamino-N-(2-(piperidin-1-yl)ethyl)nicotinamide

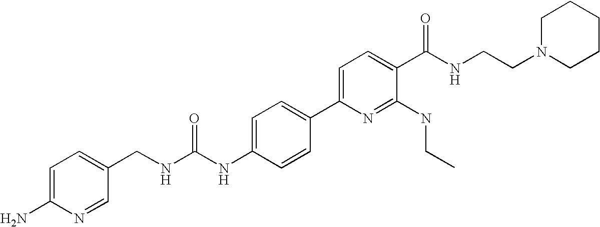

0.8 g (1.3 mm) of the compound obtained in stage 12.3 is dissolved in 20 ml of CH₂Cl₂. 11.35 ml (45.4 mm) of a 4M solution of HCl in dioxane are added. The mixture is stirred at ambient temperature for 18 h. It is concentrated. The residue is taken up in an Na₂CO₃ solution, filtered and washed with water. It is dried under vacuum over P₂O₅. 0.38 g (yd: 53%) is obtained. LC/MS; MH+ 517, tᵣ=4.94 min. ¹H NMR (250 MHz, d₆-DMSO) δ ppm: 1.21 (t, 3H), 1.29-1.61 (m, 6H), 2.32-2.47 (m, 6H), 3.24-3.39 (m, 2H), 3.44-3.58 (m, 2H), 4.10 (d, 2H), 5.84 (s, 2H), 6.42 (d, 1H), 6.51 (t, 1H), 7.09 (d, 1H), 7.35 (d, 1H), 7.50 (d, 2H), 7.87 (s, 1H), 7.94 (d, 1H), 8.01 (d, 2H), 8.35 (t, 1H), 8.42 (t, 1H), 8.71 (s, 1H).

### Example 13

### 2-Ethylamino-N-(2-piperazin-1-yl)ethyl)-6-[4-(3-(pyridin-3-ylmethyl)-ureido)phenyl]nicotinamide (compound No. 5)

¹H NMR (d₆-DMSO, 400 MHz): δ 1.22 (t, 3), 3.25 (t, 2), 3.30-3.48 (unresolved peak, 8), 3.54 (q, 2), 3.58 (t, 2), 4.47 (d, 2), 7.12 (d, 1), 7.18 (t, 1), 7.53 (d, 2), 7.86 (dd, 1), 7.98 (d, 1), 8.02 (d, 2), 8.29 (d, 1), 8.41 (unresolved peak, 2), 8.63 (t, 1), 8.74 (d, 1), 8.78 (s, 1), 9.22 (s, 1), 9.27 (unresolved peak, 3).

### Example 14

### 2-((Cyclopropylmethyl)amino)-N-methyl-6-[4-(3-(pyridin-3-ylmethyl)-ureido)phenyl]nicotinamide (compound No. 48)

¹H NMR, d₆-DMSO (300 MHz) δ 0.24 (m, 2H), 0.45 (m, 2H), 1.06 (m, 1H), 2.73 (d, J=4.1 Hz, 3H), 3.35 (t, J=6.1 Hz, 2H), 4.31 (d, J=5.1 Hz, 2H), 6.76 (t, J=6.0 Hz, 1H), 7.05 (d, J=8.0 Hz, 1H), 7.33 (t, J=5.2 Hz, 1H), 7.48 (d, J=8.6 Hz, 2H), 7.69 (d, J=7.8 Hz, 1H), 7.90 (d, J=8.1 Hz, 1H), 7.97 (d, J=8.6 Hz, 2H), 8.36 (m, 1H), 8.43 (m, 1H), 8.51 (m, 1H), 8.58 (t, J=5.1 Hz, 1H), 8.82 (s, 1H).

### Example 15

### N-Methyl-6-[4-(3-(pyridin-3-ylmethyl)ureido)phenyl]-2-(pyrrolidin-1-yl)-nicotinamide (compound No. 49)

¹H NMR, d₆-DMSO (300 MHz) δ 1.85 (m, 4H), 2.72 (d, J=4.6 Hz, 3H), 3.40 (m, 4H), 4.32 (d, J=5.8 Hz, 2H), 6.74 (t, J=5.9 Hz, 1H), 7.08 (d, J=7.7 Hz, 1H), 7.32-7.37 (m, 1H), 7.49 (m, 3H), 7.70 (m, 1H), 7.95 (d, J=8.8 Hz, 2H), 8.16 (m, 1H), 8.44 (m, 1H), 8.52 (m, 1H), 8.78 (s, 1H).

### Example 16

### N-Methyl-6-[4-(3-(pyridin-3-ylmethyl)ureido)phenyl]-2-[(tetrahydrofuran-2-ylmethyl)amino]nicotinamide (compound No. 50)

¹H NMR, d₆-DMSO (300 MHz) δ 1.59-1.63 (m, 1H), 1.80-1.93 (m, 3H), 2.74 (d, J=4.4 Hz, 3H), 3.52-3.56 (m, 1H), 3.63-3.69 (m, 2H), 3.75-3.85 (m, 1H), 4.03-4.06 (m, 1H), 4.33 (d, J=5.8 Hz, 2H), 6.76 (t, J=6.0 Hz, 1H), 7.08 (d, J=8.1 Hz, 1H), 7.33-7.38 (m, 1H), 7.50 (d, J=8.8 Hz, 2H), 7.69-7.73 (m, 1H), 7.92 (d, J=8.1 Hz, 1H), 7.99 (d, J=8.8 Hz, 2H), 8.37 (m, 1H), 8.45 (m, 1H), 8.53 (m, 1H), 8.66 (t, J=5.3 Hz, 1H), 8.83 (s, 1H).

### Example 17

### 2-(2-Methoxyethylamino)-N-methyl-6-[4-(3-(pyridin-3-ylmethyl)ureido)-phenyl]nicotinamide (compound No. 51)

¹H NMR, d₆-DMSO (300 MHz) δ 2.75 (d, J=4.3 Hz, 3H), 3.31 (s, 3H), 3.55 (t, J=5.2 Hz, 2H), 3.68 (m, 2H), 4.35 (d, J=5.7 Hz, 2H), 6.78 (t, J=5.7 Hz, 1H), 7.10 (d, J=8.1 Hz, 1H), 7.35-7.39 (m, 1H), 7.51 (d, J=8.7 Hz, 2H), 7.73 (m, 1H), 7.94 (d, J=8.1 Hz, 1H), 8.00 (d, J=8.7 Hz, 2H), 8.39 (m, 1H), 8.47 (m, 1H), 8.54 (m, 1H), 8.62 (t, J=5.0 Hz, 1H), 8.84 (s, 1H).

### Example 18

### 2-(2-Hydroxyethylamino)-N-methyl-6-[4-(3-(pyridin-3-ylmethyl)ureido)-phenyl]nicotinamide (compound No. 52)

¹H NMR, d₆-DMSO (300 MHz) δ 2.74 (d, J=4.4 Hz, 3H), 3.55-3.62 (m, 4H), 4.33 (d, J=5.8 Hz, 2H), 4.77 (t, J=4.9 Hz, 1H), 6.78 (t, J=5.8 Hz, 1H), 7.07 (d, J=8.0 Hz, 1H), 7.34-7.39 (m, 1H), 7.50 (d, J=8.8 Hz, 2H), 7.71 (m, 1H), 7.91 (d, J=8.1 Hz, 1H), 7.99 (d, J=8.8 Hz, 2H), 8.38 (m, 1H), 8.45 (m, 1H), 8.53 (m, 1H), 8.61 (m, 1H), 8.85 (s, 1H).

### Example 19

### 4′-[3-(6-Aminopyridin-3-ylmethyl)ureido]-3-(ethylamino)biphenyl-4-carboxylic acid (2-(piperidin-1-yl)ethyl)amide (Compound No. 105)

### 19.1: 4-Chloro-2-(ethylamino)benzoic acid

1.19 ml (20.94 mmol) of ethylamine as a 70% aqueous solution, 0.7 g (5.24 mmol) of potassium carbonate, 0.066 g (1.05 mmol) of copper powder and 0.42 ml (5.24 mmol) of pyridine are added to a suspension in water (20 ml) of 2 g (10.47 mmol) of 2,4-dichlorobenzoic acid. The medium is heated at 130° C. for 5 h and then stirred at ambient temperature for 48 h. The reaction medium is filtered and then a 5N HCl solution is added until the compound has precipitated. The product is filtered off and then dried in an oven in the presence of P₂O₅. 1.7 g (Yd=85%) of a white powder are obtained. LC/MS; MH⁺=200, tᵣ=8.72 min (conditions: C).

### 19.2: 4-Chloro-2-ethylamino-N-(2-(piperidin-1-yl)ethyl)benzamide

0.85 ml (6.01 mmol) of 2-(piperidin-1-yl)ethylamine, 1.96 g (6.01 mmol) of BOP and 1.54 ml (15.02 mmol) of triethylamine are added to a solution of 1 g (5.01 mmol) of 4-chloro-2-(ethylamino)benzoic acid in THF (20 ml). The mixture is stirred at ambient temperature for 12 h. The solvent is evaporated under reduced pressure. The residue is taken up in dichloromethane and washed successively with water and a saturated NaCl solution, and then the organic phase is dried on sodium sulphate. The residue is purified by flash chromatography (gradient: CH₂Cl₂ 100% to CH₂Cl₂/MeOH 90%/10%). 1.4 g (Yd=90%) of a white solid are obtained. LC/MS; MH⁺=310, tᵣ=4.33 min (conditions: A).

### 19.3: (5-{3-[3′-Ethylamino-4′-(2-(piperidin-1-yl)ethylcarbamoyl)biphenyl-4-yl]-ureidomethyl}pyridin-2-yl)carbamic acid tert-butyl ester

0.68 g (1.45 mmol) of 2-(5-{3-[4-(4,4,5,5-tetramethyl-1,3,2-dioxaborolan-2-yl)phenyl]-ureidomethyl}pyridin-2-yl)carbamic acid tert-butyl ester and 0.26 g (6.01 mmol) of potassium carbonate are added to a solution of 0.3 g (0.97 mmol) of 4-chloro-2-ethylamino-N-(2-(piperidin-1-yl)ethyl)benzamide in a toluene/water mixture (18/2 ml). The medium is stirred at ambient temperature and under argon for 30 min and then 0.034 g (0.05 mmol) of bis(di(tert-butyl)(4-dimethylaminophenyl)phospine)dichloropalladium(II) is added. The reaction medium is stirred at reflux and under argon for 5 h. The solvent is evaporated under reduced pressure. The residue is taken up in dichloromethane and successively washed with water and a saturated NaCl solution, and then the organic phase is dried over sodium sulphate. The residue is purified by flash chromatography (gradient: CH₂Cl₂ 100% to CH₂Cl₂/MeOH 80%/20%). 0.31 g (Yd=52%) of a yellow solid is obtained. LC/MS; MH⁺=616, tᵣ=4.13 min (conditions: A).

### 19.4: 4′-[3-(6-Aminopyridin-3-ylmethyl)ureido]-3-(ethylamino)biphenyl-4-carboxylic acid (2-(piperidin-1-yl)ethyl)amide

0.59 g (16.24 mmol) of a solution of hydrochloric acid in ether is added to a solution in dichloromethane (15 ml) of 0.2 g (0.32 mmol) of (5-{3-[3′-ethylamino-4′-(2-(piperidin-1-yl)-ethylcarbamoyl)biphenyl-4-yl]ureidomethyl}pyridin-2-yl)carbamic acid tert-butyl ester. The medium is stirred at ambient temperature for 2 h. The solvent is evaporated under reduced pressure. The residue is taken up in dichloromethane and successively washed with a saturated K₂CO₃ solution, water and a saturated NaCl solution, and then the organic phase is dried over sodium sulphate. The organic phases are combined and then the solvents are evaporated under reduced pressure. 0.1 g (Yd=45%) of a yellow solid is obtained. LC/MS; MH⁺=516, tᵣ=6.43 min (conditions: C). ¹H NMR (400 MHz, d₆-DMSO) δ ppm 1.23 (t, 3H), 1.34-1.78 (m, 6H), 2.47-3.07 (m, 6H), 3.17-3.27 (m, 2H), 3.40-3.56 (m, 2H), 4.11 (d, 2 H), 5.82 (s, 2H), 6.43 (d, 1H), 6.55 (t, 1H), 6.78-6.85 (m, 2H), 7.34 (d, 1H), 7.49 (d, 2H), 7.58 (d, 2H), 7.63 (d, 1H), 7.82-7.94 (m, 2H), 8.37 (br. s., 1H), 8.74 (s, 1H).

TABLE I

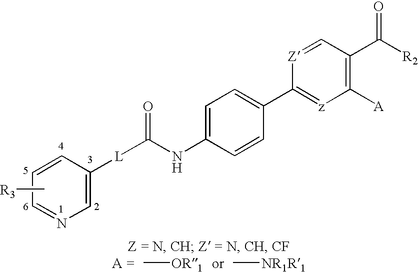

| Compound | | | | | | | | MS | LC | M.p. (° C.) | Synthetic |
|---|---|---|---|---|---|---|---|---|---|---|---|
| No. | R₃ | L | Z/Z′ | R₁ | R′₁ | R″₁ | R₂ | (MH⁺) | (Method) | or NMR | scheme |
| 1 | H | CH₂NH | N/CH | Et | H | | —NHMe | 405 | 5.61(A) | 260-263 + | Scheme 1 |
| | | | | | | | | | | NMR (Ex. | (Ex. 1) |
| | | | | | | | | | | 1) | |
| 2 | H | CH₂NH | N/CH | Et | H | | 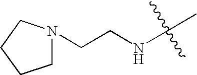 | 488 | 5.10(A) | 186-187 | Scheme 2 |
| 3 | H | CH₂NH | N/CH | H | H | | —NHMe | 377 | 4.47(A) | 234-236 + | Scheme 1 |
| | | | | | | | | | | NMR (Ex. | (Ex. 2) |
| | | | | | | | | | | 2) | |
| 4 | H | CH₂NH | N/CH | Et | H | | 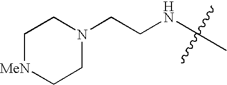 | 517 | 4.73(A) | 200-202 | Scheme 2 |
| 5 | H | CH₂NH | N/CH | Et | H | | 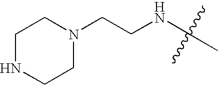 | 503 | 4.66(A) | NMR (Ex. 13) | Scheme 2- in the form of a salt with CF₃SO₃⁻ |
| 6 | H | CH₂NH | N/CH | 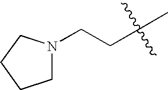 | H | | —NHMe | 474 | 4.62(A) | 166-168 | Scheme 1 |
| 7 | H | CH₂NH | N/CH | 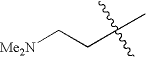 | H | | —NHMe | 488 | 4.53(A) | 163-165 + NMR (Ex. 3) | Scheme 1 (Ex. 3) |
| 8 | H | CH₂NH | N/CH | Et | H | | 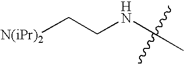 | 518 | 5.32(A) | 160-162 + NMR | Scheme 2 |
| 9 | H | CH₂NH | N/CH | Et | H | | 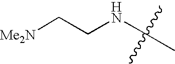 | 462 | 5.01(A) | 173-175 | Scheme 2 |
| 10 | H | CH₂NH | N/CH | Et | H | | 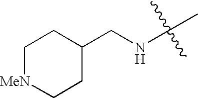 | 502 | 5.10(A) | 219-221 | Scheme 2 |
| 11 | H | CH₂NH | N/CH | 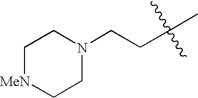 | H | | —NHMe | 503 | 4.69(A) | 180-181 | Scheme 1 |
| 12 | H | CH₂NH | N/CH | 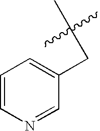 | H | | —NHMe | 468 | 5.06(A) | 224-225 | Scheme 1 |
| 13 | H | CH₂NH | N/CH | Et | H | | 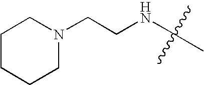 | 502 | 5.31(A) | 164-165 + NMR (Ex. 9) | Scheme 2 (Ex. 9) |
| 14 | H | CH₂NH | N/CH | 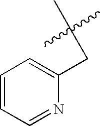 | H | | —NHMe | 468 | 4.97(A) | 236-237 | Scheme 1 |
| 15 | H | CH₂NH | N/CH | 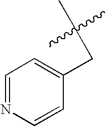 | H | | —NHMe | 468 | 4.96(A) | 218-219 + NMR (Ex. 5) | Scheme 1 (Ex. 5) |
| 16 | H | CH₂NH | N/CH | Et | H | | —NH₂ | 391 | 5.49(A) | 240-242 | Scheme 1 |
| 17 | H | CH₂NH | N/CH | 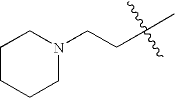 | H | | —NHMe | 488 | 4.86(A) | 187-189 | Scheme 1 |
| 18 | H | CH₂NH | N/CH | Et | H | | 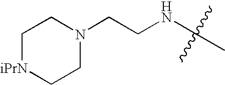 | 545 | 5.03(A) | 181-183 | Scheme 2 |
| 19 | H | CH₂NH | N/CH | 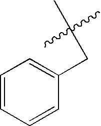 | H | | —NHMe | 467 | 6.81(A) | 231-232 | Scheme 1 |
| 20 | H | CH₂NH | N/CH | Et | H | | 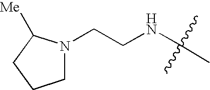 | 502 | 6.50(B) | 161-163 | Scheme 2 |
| 21 | NH₂ | CH₂NH | N/CH | Et | H | | —NHMe | 420 | 5.26(A) | 223-226 + | Scheme 3 |
| | | | | | | | | | | NMR (Ex. | (Ex. 6) |
| | | | | | | | | | | 6) | |
| 22 | H | CH₂NH | N/CH | 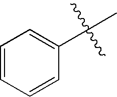 | H | | —NHMe | 453 | 6.70(A) | 234-236 + NMR (Ex. 7) | Scheme 1 (Ex. 7) |
| 23 | H | CH₂NH | N/CH | 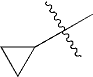 | H | | —NHMe | 417 | 5.19(A) | 210-214 | Scheme 2 |
| 24 | H | CH₂NH | N/CH | H | H | | —NH₂ | 363 | 4.18(A) | 261-263 | Scheme 1 |
| 25 | H | CH₂NH | N/CH | Et | Et | | —NHMe | 433 | 5.01(A) | 200-204 | Scheme 1 |
| 26 | H | CH₂NH | N/CH | Et | H | | —NHCH₂CH₂OH | 435 | 6.81(B) | 196-198 | Scheme 2 |
| 27 | H | CH₂NH | N/CH | Et | H | | —NHCH₂CH₂OMe | 449 | 5.78(A) | 202-204 | Scheme 2 |
| 28 | H | CH₂NH | N/CH | Et | H | | —OEt | 420 | 7.50(A) | 195-197 | Scheme 1 |
| 29 | H | CH₂O | N/CH | Et | H | | —NHMe | 406 | 6.74(A) | 234-235 | Scheme 1 |
| | | | | | | | | | | | (Ex. 8) |
| 30 | H | CH₂CH₂NH | N/CH | Et | H | | —NHMe | 419 | 5.47(A) | 203-205 | Scheme 1 |
| 31 | H | CH₂NH | N/CH | Et | H | | 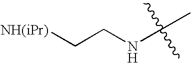 | 476 | 4.90(A) | 170-171 | Scheme 2 |
| 32 | H | CH₂NH | N/CH | Et | H | | 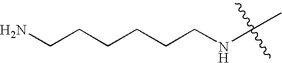 | 490 | 5.01(A) | | Scheme 2 in the form of a salt with CF₃SO₃⁻ |
| 33 | H | CH₂NH | N/CH | 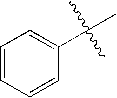 | H | | 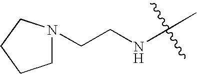 | 536 | 5.53(A) | 172-173 | Scheme 1 |
| 34 | H | CH₂NH | N/CH | Et | H | | —NHC(CH₂OH)₃ | 495 | 5.07(A) | 120 | Scheme 2 |
| 35 | H | CH₂NH | N/CH | iPr | H | | —NHMe | 419 | 6.06(A) | 211-212 | Scheme 1 |
| 36 | H | CH₂NH | N/CH | 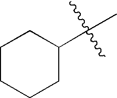 | H | | —NHMe | 459 | 6.89(A) | 132-134 | Scheme 1 |
| 37 | H | CH₂NH | N/CH | 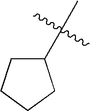 | H | | —NHMe | 445 | 6.54(A) | 128-129 | Scheme 1 |
| 38 | H | CH₂NH | N/CH | 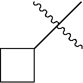 | H | | —NHMe | 431 | 6.14(A) | 214-216 | Scheme 1 |
| 39 | H | CH₂NH | N/CH | 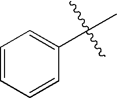 | H | | 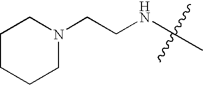 | 550 | 5.68(A) | 167-169 | Scheme 1 |
| 40 | H | CH₂NH | N/CH | Et | H | | 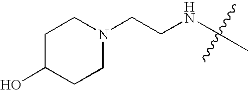 | 518 | 4.79(A) | 123-125 | Scheme 2 |
| 41 | H | CH₂NH | N/CH | Et | H | | 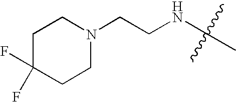 | 538 | 5.20(A) | 158-160 | Scheme 2 |
| 42 | H | CH₂NH | N/CH | Et | H | | 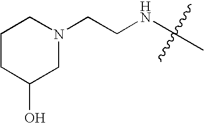 | 518 | 4.84(A) | 108-110 | Scheme 2 |
| 43 | H | CH₂NH | N/CH | Et | H | | 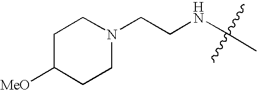 | 532 | 5.01(A) | 195-197 | Scheme 2 |
| 44 | H | CH₂NH | N/CH | 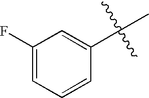 | H | | 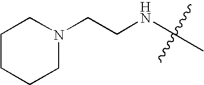 | 568 | 5.85(A) | 214-216 | Scheme 1 |
| 45 | H | CH₂NH | N/CH | 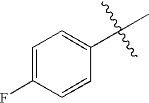 | H | | 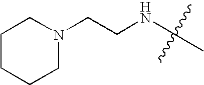 | 568 | 5.78(A) | 223-224 | Scheme 1 |
| 46 | H | CH₂NH | N/CH | 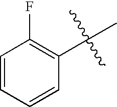 | H | | 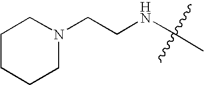 | 568 | 5.86(A) | 207-209 | Scheme 1 |
| 47 | H | CH₂NH | N/N | Et | H | | —NHMe | 406 | 4.88(A) | | Scheme 2 |
| 48 | H | CH₂NH | N/CH | 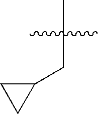 | H | | —NHMe | 431 | | NMR (Ex. 14) | Scheme 1 |
| 49 | H | CH₂NH | N/CH | 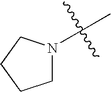 | | | —NHMe | 431 | | NMR (Ex. 15) | Scheme 1 |
| 50 | H | CH₂NH | N/CH | 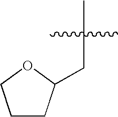 | H | | —NHMe | 461 | | NMR (Ex. 16) | Scheme 1 |
| 51 | H | CH₂NH | N/CH | —CH₂CH₂OMe | H | | —NHMe | 435 | | NMR | Scheme 1 |
| | | | | | | | | | | (Ex. 17) | |
| 52 | H | CH₂NH | N/CH | —CH₂CH₂OH | H | | —NHMe | 421 | | NMR | Scheme 1 |
| | | | | | | | | | | (Ex. 18) | |
| 53 | H | CH₂NH | N/CH | 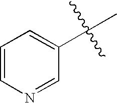 | H | | —NHMe | 454 | 4.92(A) | 213-215 | Scheme 1 |
| 54 | H | CH₂NH | N/CH | 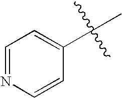 | H | | —NHMe | 454 | 4.82(A) | 320-322 | Scheme 1 |
| 55 | H | CH₂NH | CH/N | Et | H | | —NHMe | 405 | 4.56(A) | 212-215 | Scheme 1 |
| 56 | H | CH₂CH₂ | N/CH | Et | H | | —NHMe | 404 | 5.74(A) | 233-235 | Scheme 5 |
| 57 | H | CH₂NH | N/CH | 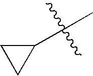 | H | | 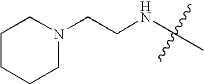 | 514 | 4.89(A) | | Scheme 2 |
| 58 | H | CH₂NH | N/CH | 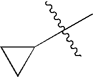 | H | | 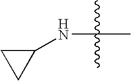 | 443 | 5.57(A) | | Scheme 2 |
| 59 | H | CH₂NH | N/CH | 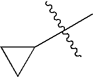 | H | | —NH(ⁿBu) | 459 | 6.47(A) | | Scheme 2 |
| 60 | H | CH₂NH | N/CH | 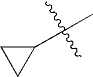 | H | | 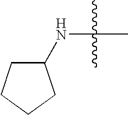 | 471 | 6.48(A) | | Scheme 2 |
| 61 | H | CH₂NH | N/CH | 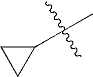 | H | | —NHEt | 431 | 5.48(A) | | Scheme 2 |
| 62 | H | CH₂NH | CH/CH | Et | H | | —NHMe | 404 | 5.58(A) | | Scheme 1 |
| 63 | H | CH₂NH | N/CH | | | —OEt | —NHMe | | | 200 + | Ex. 10 |
| | | | | | | | | | | NMR (Ex. | |
| | | | | | | | | | | 10) | |
| 64 | H | CH₂NH | N/CH | Et | H | | 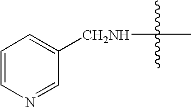 | 482 | 5.20(A) | 209-211 | Scheme 2 |
| 65 | H | CH₂NH | N/CH | Et | H | | 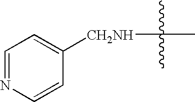 | 482 | 5.03(A) | 206 | Scheme 2 |
| 66 | H | CH₂NH | N/CH | Et | H | | 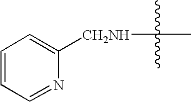 | 482 | 5.52(A) | | Scheme 2 |
| 67 | H | CH₂NH | N/CH | Et | H | | 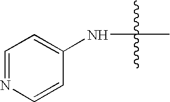 | 468 | 5.28(A) | | Scheme 2 |
| 68 | H | CH₂NH | N/CH | Et | H | | 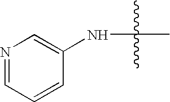 | 468 | 8.01(B) | 246-247 | Scheme 2 |
| 69 | H | CH₂NH | N/CH | Et | H | | 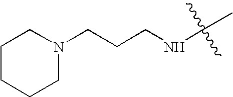 | 516 | 5.16(A) | | Scheme 2 |
| 70 | H | CH₂NH | N/CH | Et | H | | 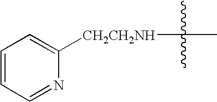 | 496 | 5.33(A) | 175-177 | Scheme 2 |
| 71 | H | CH₂NH | N/CH | Et | H | | 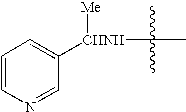 | 496 | 5.42(A) | 175-176 | Scheme 2 |
| 72 | H | CH₂NH | N/CH | Et | H | | 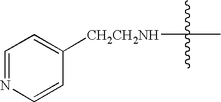 | 496 | 5.03(A) | | Scheme 2 |
| 73 | H | CH═CH | N/CH | Et | H | | —NHMe | 402 | 7.06(A) | 295-297 | Scheme 4 |
| 74 | H | CH═CH | N/CH | Et | H | | 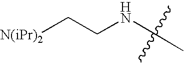 | 515 | 7.63(C) | 224-226 | Scheme 4 |
| 75 | H | CH═CH | N/CH | Et | H | | 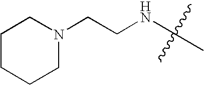 | 499 | 5.93(A) | 235-237 | Scheme 4 |
| 76 | H | CH₂NH | N/CH | Et | H | | 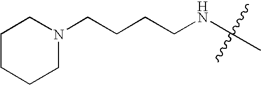 | 530 | 5.04(A) | | Scheme 2 |
| 77 | H | CH₂NH | N/CH | Et | H | | 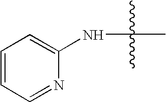 | 468 | 6.51(A) | | Scheme 2 |
| 78 | H | CH₂NH | N/CF | Et | H | | —NHMe | 423 | 6.09(A) | | Scheme 2 |
| 79 | H | CH₂NH | N/CF | Et | H | | 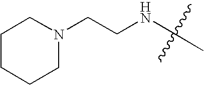 | 520 | 3.09(E) | | Scheme 2 |
| 80 | H | CH₂NH | N/N | Et | H | | 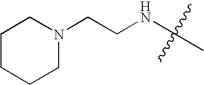 | 503 | 4.71(A) | NMR (Ex. 11) | Scheme 2 (Ex. 11) |
| 81 | 6-NH₂ | CH₂NH | N/CH | Et | H | | 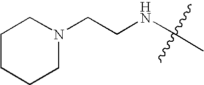 | 517 | 4.94(A) | NMR (Ex. 12) | Scheme 3 (Ex. 12) |
| 82 | 2-F | CH₂NH | N/CH | Et | H | | 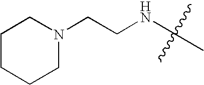 | 520 | 6.1(A) | | Scheme 3 |
| 83 | 6-Me | CH₂NH | N/CH | Et | H | | 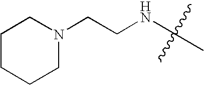 | 516 | 4.99(A) | | Scheme 3 |
| 84 | 2,5,6-F | CH₂NH | N/CH | Et | H | | 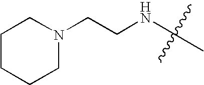 | 556 | 6.8(A) | | Scheme 3 |
| 85 | 5-Me | CH₂NH | N/CH | Et | H | | 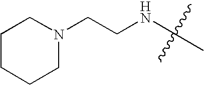 | 516 | 5.16(A) | | Scheme 3 |
| 86 | 2-OMe | CH₂NH | N/CH | Et | H | | 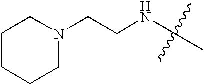 | 532 | 6.34(A) | | Scheme 3 |
| 87 | 5-NH₂ | CH₂NH | N/CH | Et | H | | 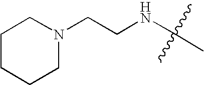 | 517 | 4.88(A) | | Scheme 3 |
| 88 | 5-F | CH₂NH | N/CH | Et | H | | 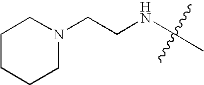 | 520 | 6.01 | | Scheme 3 |
| 89 | 6-F | CH₂NH | N/CH | Et | H | | 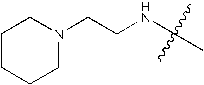 | 520 | 6.14(A) | | Scheme 3 |
| 90 | 6-NMe₂ | CH₂NH | N/CH | Et | H | | 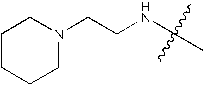 | 545 | 5.07(A) | | Scheme 3 |
| 91 | 6-CN | CH₂NH | N/CH | Et | H | | 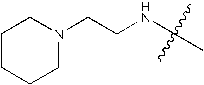 | 527 | 7.42(C) | | Scheme 3 |
| 92 | 6-NH-Boc | CH₂NH | N/CH | Et | H | | 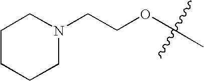 | 618 | 1.09(D) | | Scheme 3′ |
| 93 | 6-NH₂ | CH₂NH | N/CH | Et | H | | 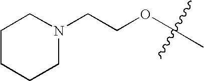 | 518 | 1.35(D) | | Scheme 3′ |
| 94 | 6-NH-Me | CH₂NH | N/CH | Et | H | | 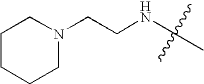 | 531 | 0.74(D) | | Scheme 3 in the trihydrochlor- ide form |
| 95 | 5-Me, 6- NH₂ | CH₂NH | N/CH | Et | H | | 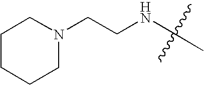 | 531 | 0.75(D) | | Scheme 3 |
| 96 | 6-NH₂ | CH₂NH | N/CH | Et | H | | 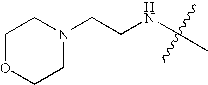 | 519 | 0.65(D) | | Scheme 2 |
| 97 | 6-NH₂ | CH₂NH | N/CH | Et | H | | 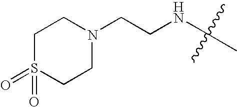 | 567 | 0.7(D) | | Scheme 2 |
| 98 | 6-NH₂ | CH₂NH | N/CH | 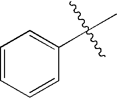 | H | | 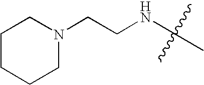 | 565 | 0.95(D) | | Scheme 3 |
| 99 | 6-NH₂ | CH₂NH | N/CH | 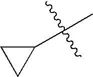 | H | | 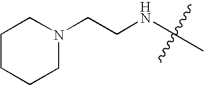 | 529 | 0.65(D) | | Scheme 3 |
| 100 | 6-NH₂ | CH₂NH | N/CH | Et | H | | 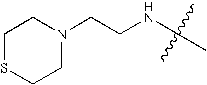 | 535 | 0.71(D) | | Scheme 2 |
| 101 | 6- NHCOMe | CH₂NH | N/CH | Et | H | | 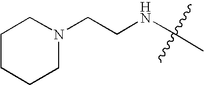 | 559 | 0.71(D) | | Scheme 3 |
| 102 | 6-NH₂ | CH═CG | N/CH | Et | H | | 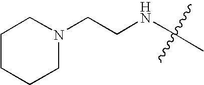 | 514 | 0.77(D) | | Scheme 5 |
| 103 | 6-NH₂ | CH₂NH | N/CH | Et | H | | 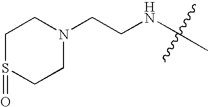 | 551 | 0.58(D) | | Scheme 2 |
| 104 | 6-NH₂ | CH₂NH | N/CH | Et | H | | —NHCH₂CH₂NH-iPr | 491 | 5.12(A) | | Scheme 1 in the trihydrochlor- ide form |
| 105 | 6-NH₂ | CH₂NH | CH/CH | Et | H | | 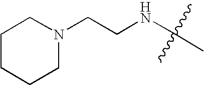 | 516 | 6.43(A) | NMR (Ex. 19) | Scheme 1 (Ex. 19) |
| 106 | 5-Me, 6- NH₂ | CH₂NH | CH/CH | Et | H | | 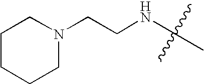 | 530 | 0.77(D) | | Scheme 3 |
| 107 | 6-NHCO- iPr | CH₂NH | N/CH | Et | H | | 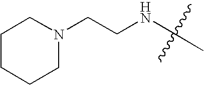 | 587 | 0.88(D) | | Scheme 3 |
| 108 | 6-NH-iPr | CH₂NH | N/CH | Et | H | | 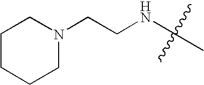 | 559 | 0.79(D) | | Scheme 3 |
| 109 | 6-NH-Et | CH₂NH | N/CH | Et | H | | 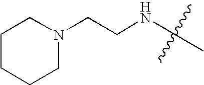 | 545 | 0.75(D) | | Scheme 3 |
| 110 | 6- NHCOO- tBu | CH₂NH | N/CH | Et | H | | 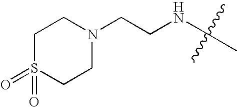 | 667 | 0.94(D) | | Scheme 3 |
| 111 | 6- NHCOO- tBu | CH₂NH | N/CH | Et | H | | 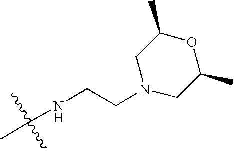 | 647 | 0.99(D) | | Scheme 3 |
| 112 | 6-NH₂ | CH₂NH | N/CH | 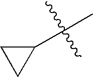 | H | | 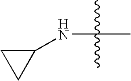 | 458 | 0.75(D) | | Scheme 2 |
| 113 | 6-NH₂ | CH₂NH | N/CH | 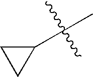 | H | | —NH-nBu | 474 | 0.96(D) | | Scheme 3 |
| 114 | 6-NH₂ | CH₂NH | N/CH | Et | H | | 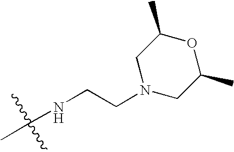 | 547 | 0.73(D) | | Scheme 3 |
| 115 | 6-NH₂ | CH₂NH | N/CH | Et | H | | —NHCH₂CH₂OH | 450 | 0.63(D) | | Scheme 2 |
| 116 | 6-NH₂ | CH₂NH | N/CH | 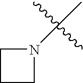 | | | 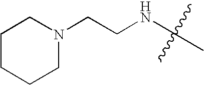 | 529 | 5.91(C) | | Scheme 3 |
| 117 | 6-NH₂ | CH₂NH | N/CH | 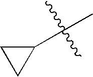 | H | | 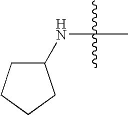 | 486 | 0.98(D) | | Scheme 2 |
| 118 | 6-NH₂ | CH₂NH | N/CH | 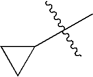 | H | | —NH-Et | 446 | 0.74(D) | | Scheme 2 |
| 119 | 6-NH₂ | CH₂NH | CH/CH | 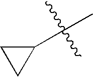 | H | | 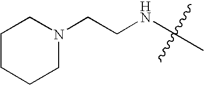 | 528 | 0.85(D) | | Scheme 1 in the trihydrochlor- ide form |
| 120 | 6-NH₂ | CH₂NH | N/CH | Et | H | | —NHCH₂CH₂OMe | 464 | 0.76(D) | | Scheme 3 |
| 121 | 6-NH₂ | CH₂NH | CH/CH | Et | H | | 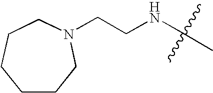 | 531 | 0.76(D) | | Scheme 3 |
| 122 | 6-NH₂ | CH₂NH | CH/CH | Et | H | | 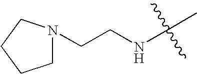 | 503 | 0.65(D) | | Scheme 3 |
| 123 | 6-OH | CH₂NH | N/CH | Et | H | | 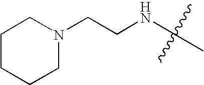 | 518 | 5.52(D) | | Scheme 3 |
| 124 | 2-NH₂ | CH₂NH | N/CH | Et | H | | 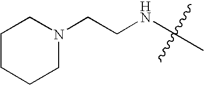 | 517 | 5.0(D) | | Scheme 3 |
| 125 | 6-NH₂ | CH₂NH | N/CH | Et | H | | 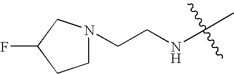 | 521 | 0.65(D) | | Scheme 2 |
| 126 | 6-NH₂ | CH₂NH | N/N | Et | H | | 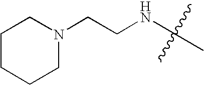 | 518 | 0.64(D) | | Scheme 2 |
| 127 | 6-NH₂ | CH₂NH | N/N | 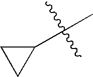 | H | | 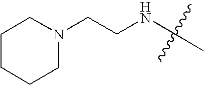 | 530 | 0.67(D) | | Scheme 2 |

| | | | | | | | | |
|---|---|---|---|---|---|---|---|---|
| 128 | 6-NH₂ | CH₂NH | N/CH | 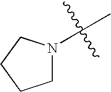 | 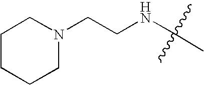 | 543 | 6.19(C) | Scheme 3 |
| 129 | 6-NH₂ | CH₂NH | N/CH | 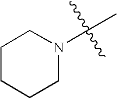 | 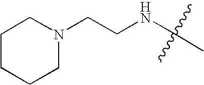 | 557 | 0.71(D) | Scheme 3 |

nBu: n-butyl;
tBu: tert-butyl;
Pr: isopropyl
for R₃: 6-NH₂ means —NH₂ in the 6 position on the pyridine ring as indicated;
2-F means —F in the 5 position on the pyridine ring

The compounds in Table I have as chemical name (obtained from the Autonom® software):
2-Ethylamino-N-methyl-6-[4-(3-pyridin-3-ylmethyl-ureido)-phenyl]-nicotinamide (Compound n° 1)
2-Ethylamino-6-[4-(3-pyridin-3-ylmethyl-ureido)-phenyl]-N-(2-pyrrolidin-1-yl-ethyl)-nicotinamide (n° 2)
2-Amino-N-methyl-6-[4-(3-pyridin-3-ylmethyl-ureido)-phenyl]-nicotinamide (n° 3)
2-Ethylamino-N-[2-(4-methyl-piperazin-1-yl)-ethyl]-6-[4-(3-pyridin-3-ylmethyl-ureido)-phenyl]-nicotinamide (n° 4)
2-Ethylamino-N-(2-piperazin-1-yl-ethyl)-6-[4-(3-pyridin-3-ylmethyl-ureido)-phenyl]-nicotinamide (n° 5)
N-Methyl-6-[4-(3-pyridin-3-ylmethyl-ureido)-phenyl]-2-(2-pyrrolidin-1-yl-ethylamino)-nicotinamide (n° 6)
2-(2-Dimethylamino-ethylamino)-N-methyl-6-[4-(3-pyridin-3-ylmethyl-ureido)-phenyl]-nicotinamide (n° 7)
N-(2-Diisopropylamino-ethyl)-2-ethylamino-6-[4-(3-pyridin-3-ylmethyl-ureido)-phenyl]-nicotinamide (n° 8)
N-(2-Dimethylamino-ethyl)-2-ethylamino-6-[4-(3-pyridin-3-ylmethyl-ureido)-phenyl]-nicotinamide (n° 9)
2-Ethylamino-N-(1-methyl-piperidin-4-ylmethyl)-6-[4-(3-pyridin-3-ylmethyl-ureido)-phenyl]-nicotinamide (n° 10)
N-Methyl-2-[2-(4-methyl-piperazin-1-yl)-ethylamino]-6-[4-(3-pyridin-3-ylmethyl-ureido)-phenyl]-nicotinamide (n° 11)
N-Methyl-2-[(pyridin-3-ylmethyl)-amino]-6-[4-(3-pyridin-3-ylmethyl-ureido)-phenyl]-nicotinamide (n° 12)
2-Ethylamino-N-(2-piperidin-1-yl-ethyl)-6-[4-(3-pyridin-3-ylmethyl-ureido)-phenyl]-nicotinamide (n° 13)
N-Methyl-2-[(pyridin-2-ylmethyl)-amino]-6-[4-(3-pyridin-3-ylmethyl-ureido)-phenyl]-nicotinamide (n° 14)
N-Methyl-2-[(pyridin-4-ylmethyl)-amino]-6-[4-(3-pyridin-3-ylmethyl-ureido)-phenyl]-nicotinamide (n° 15)
2-Ethylamino-6-[4-(3-pyridin-3-ylmethyl-ureido)-phenyl]-nicotinamide (n° 16)
N-Methyl-2-(2-piperidin-1-yl-ethylamino)-6-[4-(3-pyridin-3-ylmethyl-ureido)-phenyl]-nicotinamide (n° 17)
2-Ethylamino-N-[2-(4-isopropyl-piperazin-1-yl)-ethyl]-6-[4-(3-pyridin-3-ylmethyl-ureido)-phenyl]-nicotinamide (n° 18)
2-Benzylamino-N-methyl-6-[4-(3-pyridin-3-ylmethyl-ureido)-phenyl]-nicotinamide (n° 19)
2-Ethylamino-N-[2-(2-methyl-pyrrolidin-1-yl)-ethyl]-6-[4-(3-pyridin-3-ylmethyl-ureido)-phenyl]-nicotinamide (n° 20)
6-{-4-[3-(6-Amino-pyridin-3-ylmethyl)-ureido]-phenyl}-2-ethylamino-N-methyl-nicotinamide (n° 21)
N-Methyl-2-phenylamino-6-[4-(3-pyridin-3-ylmethyl-ureido)-phenyl]-nicotinamide (n° 22)
2-Cyclopropylamino-N-methyl-6-[4-(3-pyridin-3-ylmethyl-ureido)-phenyl]-nicotinamide (n° 23)
2-Amino-6-[4-(3-pyridin-3-ylmethyl-ureido)-phenyl]-nicotinamide (n° 24)
2-Diethylamino-N-methyl-6-[4-(3-pyridin-3-ylmethyl-ureido)-phenyl]-nicotinamide (n° 25)
2-Ethylamino-N-(2-hydroxy-ethyl)-6-[4-(3-pyridin-3-ylmethyl-ureido)-phenyl]-nicotinamide (n° 26)
2-Ethylamino-N-(2-methoxy-ethyl)-6-[4-(3-pyridin-3-ylmethyl-ureido)-phenyl]-nicotinamide (n° 27)
2-Ethylamino-6-[4-(3-pyridin-3-ylmethyl-ureido)-phenyl]-nicotinic acid ethyl ester (n° 28)
[4-(6-Ethylamino-5-methylcarbamoyl-pyridin-2-yl)-phenyl]-carbamic acid pyridin-3-ylmethyl ester (n° 29)
2-Ethylamino-N-methyl-6-{-4-[3-(2-pyridin-3-yl-ethyl)-ureido]-phenyl}-nicotinamide (n° 30)
2-Ethylamino-N-(2-isopropylamino-ethyl)-6-[4-(3-pyridin-3-ylmethyl-ureido)-phenyl]-nicotinamide (n° 31)
N-(6-Amino-hexyl)-2-ethylamino-6-[4-(3-pyridin-3-ylmethyl-ureido)-phenyl]-nicotinamide (n° 32)
2-Phenylamino-6-[4-(3-pyridin-3-ylmethyl-ureido)-phenyl]-N-(2-pyrrolidin-1-yl-ethyl)-nicotinamide (n° 33)
2-Ethylamino-N-(2-hydroxy-1,1-bis-hydroxymethyl-ethyl)-6-[4-(3-pyridin-3-ylmethyl-ureido)-phenyl]-nicotinamide (n° 34)
2-Isopropylamino-N-methyl-6-[4-(3-pyridin-3-ylmethyl-ureido)-phenyl]-nicotinamide (n° 35)
2-Cyclohexylamino-N-methyl-6-[4-(3-pyridin-3-ylmethyl-ureido)-phenyl]-nicotinamide (n° 36)
2-Cyclopentylamino-N-methyl-6-[4-(3-pyridin-3-ylmethyl-ureido)-phenyl]-nicotinamide (n° 37)
2-Cyclobutylamino-N-methyl-6-[4-(3-pyridin-3-ylmethyl-ureido)-phenyl]-nicotinamide (n° 38)
2-Phenylamino-N-(2-piperidin-1-yl-ethyl)-6-[4-(3-pyridin-3-ylmethyl-ureido)-phenyl]-nicotinamide (n° 39)
2-Ethylamino-N-[2-(4-hydroxy-piperidin-1-yl)-ethyl]-6-[4-(3-pyridin-3-ylmethyl-ureido)-phenyl]-nicotinamide (n° 40)
N-[2-(4,4-Difluoro-piperidin-1-yl)-ethyl]-2-ethylamino-6-[4-(3-pyridin-3-ylmethyl-ureido)-phenyl]-nicotinamide (n° 41)
2-Ethylamino-N-[2-(3-hydroxy-piperidin-1-yl)-ethyl]-6-[4-(3-pyridin-3-ylmethyl-ureido)-phenyl]-nicotinamide (n° 42)
2-Ethylamino-N-[2-(4-methoxy-piperidin-1-yl)-ethyl]-6-[4-(3-pyridin-3-ylmethyl-ureido)-phenyl]-nicotinamide (n° 43)
2-(3-Fluoro-phenylamino)-N-(2-piperidin-1-yl-ethyl)-6-[4-(3-pyridin-3-ylmethyl-ureido)-phenyl]-nicotinamide (n° 44)
2-(4-Fluoro-phenylamino)-N-(2-piperidin-1-yl-ethyl)-6-[4-(3-pyridin-3-ylmethyl-ureido)-phenyl]-nicotinamide (n° 45)
2-(2-Fluoro-phenylamino)-N-(2-piperidin-1-yl-ethyl)-6-[4-(3-pyridin-3-ylmethyl-ureido)-phenyl]-nicotinamide (n° 46)
4-Ethylamino-2-[4-(3-pyridin-3-ylmethyl-ureido)-phenyl]-pyrimidine-5-carboxylic acid methylamide (n° 47)
2-(Cyclopropylmethyl-amino)-N-methyl-6-[4-(3-pyridin-3-ylmethyl-ureido)-phenyl]-nicotinamide (n° 48)
N-Methyl-6-[4-(3-pyridin-3-ylmethyl-ureido)-phenyl]-2-pyrrolidin-1-yl-nicotinamide (n° 49)
N-Methyl-6-[4-(3-pyridin-3-ylmethyl-ureido)-phenyl]-2-[(tetrahydro-furan-2-ylmethyl)-amino]-nicotinamide (n° 50)
2-(2-Methoxy-ethylamino)-N-methyl-6-[4-(3-pyridin-3-ylmethyl-ureido)-phenyl]-nicotinamide (n° 51)
2-(2-Hydroxy-ethylamino)-N-methyl-6-[4-(3-pyridin-3-ylmethyl-ureido)-phenyl]-nicotinamide (n° 52)
N-Methyl-2-(pyridin-3-ylamino)-6-[4-(3-pyridin-3-ylmethyl-ureido)-phenyl]-nicotinamide (n° 53)
N-Methyl-2-(pyridin-4-ylamino)-6-[4-(3-pyridin-3-ylmethyl-ureido)-phenyl]-nicotinamide (n° 54)
4-Ethylamino-N-methyl-6-[4-(3-pyridin-3-ylmethyl-ureido)-phenyl]-nicotinamide (n° 55)
2-Ethylamino-N-methyl-6-[4-(3-pyridin-3-yl-propionylamino)-phenyl]-nicotinamide (n° 56)
2-Cyclopropylamino-N-(2-piperidin-1-yl-ethyl)-6-[4-(3-pyridin-3-ylmethyl-ureido)-phenyl]-nicotinamide (n° 57)
N-Cyclopropyl-2-cyclopropylamino-6-[4-(3-pyridin-3-ylmethyl-ureido)-phenyl]-nicotinamide (n° 58)
N-Butyl-2-cyclopropylamino-6-[4-(3-pyridin-3-ylmethyl-ureido)-phenyl]-nicotinamide (n° 59)
N-Cyclopentyl-2-cyclopropylamino-6-[4-(3-pyridin-3-ylmethyl-ureido)-phenyl]-nicotinamide (n° 60)
2-Cyclopropylamino-N-ethyl-6-[4-(3-pyridin-3-ylmethyl-ureido)-phenyl]-nicotinamide (n° 61)
3-Ethylamino-4′-(3-pyridin-3-ylmethyl-ureido)-biphenyl-4-carboxylic acid methylamide (n° 62)
2-Ethoxy-N-methyl-6-[4-(3-pyridin-3-ylmethyl-ureido)-phenyl]-nicotinamide (n° 63)
2-Ethylamino-N-pyridin-3-ylmethyl-6-[4-(3-pyridin-3-ylmethyl-ureido)-phenyl]-nicotinamide (n° 64)
2-Ethylamino-N-pyridin-4-ylmethyl-6-[4-(3-pyridin-3-ylmethyl-ureido)-phenyl]-nicotinamide (n° 65)
2-Ethylamino-N-pyridin-2-ylmethyl-6-[4-(3-pyridin-3-ylmethyl-ureido)-phenyl]-nicotinamide (n° 66)
2-Ethylamino-N-pyridin-4-yl-6-[4-(3-pyridin-3-ylmethyl-ureido)-phenyl]-nicotinamide (n° 67)
2-Ethylamino-N-pyridin-3-yl-6-[4-(3-pyridin-3-ylmethyl-ureido)-phenyl]-nicotinamide (n° 68)
2-Ethylamino-N-(3-piperidin-1-yl-propyl)-6-[4-(3-pyridin-3-ylmethyl-ureido)-phenyl]-nicotinamide (n° 69)
2-Ethylamino-N-(2-pyridin-2-yl-ethyl)-6-[4-(3-pyridin-3-ylmethyl-ureido)-phenyl]-nicotinamide (n° 70)
2-Ethylamino-N-(1-pyridin-3-yl-ethyl)-6-[4-(3-pyridin-3-ylmethyl-ureido)-phenyl]-nicotinamide (n° 71)
2-Ethylamino-N-(2-pyridin-4-yl-ethyl)-6-[4-(3-pyridin-3-ylmethyl-ureido)-phenyl]-nicotinamide (n° 72)
2-Ethylamino-N-methyl-6-[4-((E)-3-pyridin-3-yl-acryloylamino)-phenyl]-nicotinamide (n° 73)
N-(2-Diisopropylamino-ethyl)-2-ethylamino-6-[4-((E)-3-pyridin-3-yl-acryloylamino)-phenyl]-nicotinamide (n° 74)
2-Ethylamino-N-(2-piperidin-1-yl-ethyl)-6-[4-((E)-3-pyridin-3-yl-acryloylamino)-phenyl]-nicotinamide (n° 75)
2-Ethylamino-N-(4-piperidin-1-yl-butyl)-6-[4-(3-pyridin-3-ylmethyl-ureido)-phenyl]-nicotinamide (N° 76)
2-Ethylamino-N-pyridin-2-yl-6-[4-(3-pyridin-3-ylmethyl-ureido)-phenyl]-nicotinamide (n° 77)
2-Ethylamino-5-fluoro-N-methyl-6-[4-(3-pyridin-3-ylmethyl-ureido)-phenyl]-nicotinamide (n° 78)
2-Ethylamino-5-fluoro-N-(2-piperidin-1-yl-ethyl)-6-[4-(3-pyridin-3-ylmethyl-ureido)-phenyl]-nicotinamide (n° 79)
4-Ethylamino-2-[4-(3-pyridin-3-ylmethyl-ureido)-phenyl]-pyrimidine-5-carboxylic acid (2-piperidin-1-yl-ethyl)-amide (n° 80)
6-{-4-[3-(6-Amino-pyridin-3-ylmethyl)-ureido]-phenyl}-2-ethylamino-N-(2-piperidin-1-yl-ethyl)-nicotinamide (n° 81)
2-Ethylamino-6-{4-[3-(2-fluoro-pyridin-3-ylmethyl)-ureido]-phenyl}-N-(2-piperidin-1-yl-ethyl)-nicotinamide (n° 82)
2-Ethylamino-6-{-4-[3-(6-methyl-pyridin-3-ylmethyl)-ureido]-phenyl}-N-(2-piperidin-1-yl-ethyl)-nicotinamide (n° 83)
2-Ethylamino-N-(2-piperidin-1-yl-ethyl)-6-{-4-[3-(2,5,6-trifluoro-pyridin-3-ylmethyl)-ureido]-phenyl}-nicotinamide (n° 84)
2-Ethylamino-6-{-4-[3-(5-methyl-pyridin-3-ylmethyl)-ureido]-phenyl}-N-(2-piperidin-1-yl-ethyl)-nicotinamide (n° 85)
2-Ethylamino-6-{4-[3-(2-methoxy-pyridin-3-ylmethyl)-ureido]-phenyl}-N-(2-piperidin-1-yl-ethyl)-nicotinamide (n° 86)
6-{-4-[3-(5-Amino-pyridin-3-ylmethyl)-ureido]-phenyl}-2-ethylamino-N-(2-piperidin-1-yl-ethyl)-nicotinamide (n° 87)
2-Ethylamino-6-{4-[3-(5-fluoro-pyridin-3-ylmethyl)-ureido]-phenyl}-N-(2-piperidin-1-yl-ethyl)-nicotinamide (n° 88)
2-Ethylamino-6-{4-[3-(6-fluoro-pyridin-3-ylmethyl)-ureido]-phenyl}-N-(2-piperidin-1-yl-ethyl)-nicotinamide (n° 89)
6-{-4-[3-(6-Dimethylamino-pyridin-3-ylmethyl)-ureido]-phenyl}-2-ethylamino-N-(2-piperidin-1-yl-ethyl)-nicotinamide (n° 90)
6-{-4-[3-(6-Cyano-pyridin-3-ylmethyl)-ureido]-phenyl}-2-ethylamino-N-(2-piperidin-1-yl-ethyl)-nicotinamide (n° 91)
6-{-4-[3-(6-tert-Butoxycarbonylamino-pyridin-3-ylmethyl)-ureido]-phenyl}-2-ethylamino-nicotinic acid 2-piperidin-1-yl-ethyl ester (n° 92)
6-{-4-[3-(6-Amino-pyridin-3-ylmethyl)-ureido]-phenyl}-2-ethylamino-nicotinic acid 2-piperidin-1-yl-ethyl ester (n° 93)
2-Ethylamino-6-{-4-[3-(6-methylamino-pyridin-3-ylmethyl)-ureido]-phenyl}-N-(2-piperidin-1-yl-ethyl)-nicotinamide (n° 94)
6-{-4-[3-(6-Amino-5-methyl-pyridin-3-ylmethyl)-ureido]-phenyl}-2-ethylamino-N-(2-piperidin-1-yl-ethyl)-nicotinamide (n° 95)
6-{-4-[3-(6-Amino-pyridin-3-ylmethyl)-ureido]-phenyl}-2-ethylamino-N-(2-morpholin-4-yl-ethyl)-nicotinamide (n° 96)
6-{-4-[3-(6-Amino-pyridin-3-ylmethyl)-ureido]-phenyl}-N-[2-(1,1-dioxo-1-thiomorpholin-4-yl)-ethyl]-2-ethylamino-nicotinamide (n° 97)
6-{-4-[3-(6-Amino-pyridin-3-ylmethyl)-ureido]-phenyl}-2-phenylamino-N-(2-piperidin-1-yl-ethyl)-nicotinamide (n° 98)
6-{-4-[3-(6-Amino-pyridin-3-ylmethyl)-ureido]-phenyl}-2-cyclopropylamino-N-(2-piperidin-1-yl-ethyl)-nicotinamide (n° 99)
6-{-4-[3-(6-Amino-pyridin-3-ylmethyl)-ureido]-phenyl}-2-ethylamino-N-(2-thiomorpholin-4-yl-ethyl)-nicotinamide (n° 100)
6-{-4-[3-(6-Acetylamino-pyridin-3-ylmethyl)-ureido]-phenyl}-2-ethylamino-N-(2-piperidin-1-yl-ethyl)-nicotinamide (n° 101)
6-{4-[(E)-3-(6-Amino-pyridin-3-yl)-acryloylamino]-phenyl}-2-ethylamino-N-(2-piperidin-1-yl-ethyl)-nicotinamide (n° 102)
6-{-4-[3-(6-Amino-pyridin-3-ylmethyl)-ureido]-phenyl}-2-ethylamino-N-[2-(1-oxo-1-thiomorpholin-4-yl)-ethyl]-nicotinamide (n° 103)
6-{-4-[3-(6-Amino-pyridin-3-ylmethyl)-ureido]-phenyl}-2-ethylamino-N-(2-isopropylamino-ethyl)-nicotinamide (n° 104)
4′-[3-(6-Amino-pyridin-3-ylmethyl)-ureido]-3-ethylamino-biphenyl-4-carboxylic acid (2-piperidin-1-yl-ethyl)-amide (n° 105)
4′-[3-(6-Amino-5-methyl-pyridin-3-ylmethyl)-ureido]-3-ethylamino-biphenyl-4-carboxylic acid (2-piperidin-1-yl-ethyl)amide (n° 106)
2-Ethylamino-6-{4-[3-(6-isobutyrylamino-pyridin-3-ylmethyl)-ureido]-phenyl}-N-(2-piperidin-1-yl-ethyl)-nicotinamide (n° 107)
2-Ethylamino-6-{-4-[3-(6-isopropylamino-pyridin-3-ylmethyl)-ureido]-phenyl}-N-(2-piperidin-1-yl-ethyl)-nicotinamide (n° 108)
2-Ethylamino-6-{4-[3-(6-ethylamino-pyridin-3-ylmethyl)-ureido]-phenyl}-N-(2-piperidin-1-yl-ethyl)-nicotinamide (n° 109)
{5-[3-(4-{5-[2-(1,1-Dioxo-1-thiomorpholin-4-yl)-ethylcarbamoyl]-6-ethylamino-pyridin-2-yl}-phenyl)-ureidomethyl]-pyridin-2-yl}-carbamic acid tert-butyl ester (n° 110)
{5-[3-(4-{5-[2-(cis-2,6-Dimethyl-morpholin-4-yl)-ethylcarbamoyl]-6-ethylamino-pyridin-2-yl}-phenyl)-ureidomethyl]-pyridin-2-yl}-carbamic acid tert-butyl ester (n° 111)
6-{-4-[3-(6-Amino-pyridin-3-ylmethyl)-ureido]-phenyl}-N-cyclopropyl-2-cyclopropylamino-nicotinamide (n° 112)
6-{-4-[3-(6-Amino-pyridin-3-ylmethyl)-ureido]-phenyl}-N-butyl-2-cyclopropylamino-nicotinamide (n° 113)
6-{-4-[3-(6-Amino-pyridin-3-ylmethyl)-ureido]-phenyl}-N-[2-(cis-2,6-dimethyl-morpholin-4-yl)-ethyl]-2-ethylamino-nicotinamide (n° 114)
6-{-4-[3-(6-Amino-pyridin-3-ylmethyl)-ureido]-phenyl}-2-ethylamino-N-(2-hydroxy-ethyl)-nicotinamide (n° 115)
6-{-4-[3-(6-Amino-pyridin-3-ylmethyl)-ureido]-phenyl}-2-azetidin-1-yl-N-(2-piperidin-1-yl-ethyl)-nicotinamide (n° 116)
6-{-4-[3-(6-Amino-pyridin-3-ylmethyl)-ureido]-phenyl}-N-cyclopentyl-2-cyclopropylamino-nicotinamide (n° 117)
6-{-4-[3-(6-Amino-pyridin-3-ylmethyl)-ureido]-phenyl}-2-cyclopropylamino-N-ethyl-nicotinamide (n° 118)
4′-[3-(6-Amino-pyridin-3-ylmethyl)-ureido]-3-cyclopropylamino-biphenyl-4-carboxylic acid (2-piperidin-1-yl-ethyl)-amide (n° 119)
6-{-4-[3-(6-Amino-pyridin-3-ylmethyl)-ureido]-phenyl}-2-ethylamino-N-(2-methoxy-ethyl)-nicotinamide (n° 120)
6-{-4-[3-(6-Amino-pyridin-3-ylmethyl)-ureido]-phenyl}-N-(2-azepan-1-yl-ethyl)-2-ethylamino-nicotinamide (n° 121)
6-{-4-[3-(6-Amino-pyridin-3-ylmethyl)-ureido]-phenyl}-2-ethylamino-N-(2-pyrrolidin-1-yl-ethyl)-nicotinamide (n° 122)
2-Ethylamino-6-{4-[3-(6-oxo-1,6-dihydro-pyridin-3-ylmethyl)-ureido]-phenyl}-N-(2-piperidin-1-yl-ethyl)-nicotinamide-n° 123)
6-{-4-[3-(2-Amino-pyridin-3-ylmethyl)-ureido]-phenyl}-2-ethylamino-N-(2-piperidin-1-yl-ethyl)-nicotinamide (n° 124)
6-{-4-[3-(6-Amino-pyridin-3-ylmethyl)-ureido]-phenyl}-2-ethylamino-N-[2-(3-fluoro-pyrrolidin-1-yl)-ethyl]-nicotinamide (n° 125)
2-{-4-[3-(6-Amino-pyridin-3-ylmethyl)-ureido]-phenyl}-4-ethylamino-pyrimidine-5-carboxylic acid (2-piperidin-1-yl-ethyl)amide (n° 126)
2-{-4-[3-(6-Amino-pyridin-3-ylmethyl)-ureido]-phenyl}-4-cyclopropylamino-pyrimidine-5-carboxylic acid (2-piperidin-1-yl-ethyl)amide (n° 127)
6-{-4-[3-(6-Amino-pyridin-3-ylmethyl)-ureido]-phenyl}-N-(2-piperidin-1-yl-ethyl)-2-pyrrolidin-1-yl-nicotinamide (n° 128) and
6′-{-4-[3-(6-Amino-pyridin-3-ylmethyl)-ureido]-phenyl}-3,4,5,6-tetrahydro-2H-[1,2′]bipyridinyl-3′-carboxylic acid (2-piperidin-1-yl-ethyl)amide (n° 129)

The compounds described in Table I have formed the subject of pharmacological trials which make it possible to determine the anticancer activity. They were tested in vitro on the following tumour lines: HCT116 (ATCC-CCL247) and PC3 (ATCC-CRL1435). The cell proliferation and viability were determined in a test using 3-(4,5-dimethylthiazol-2-yl)-5-(3-carboxymethoxyphenyl)-2-(4-sulphophenyl)-2H-tetrazolium (MTS) according to Fujishita T. et al., Oncology, 2003, 64(4), 399-406. In this test, the mitochondrial capacity of the living cells to convert MTS to a coloured compound after incubating the test compound for 72 hours is measured. The concentration of compound which results in 50% loss of cell proliferation and viability is recorded as IC₅₀.

TABLE II

| Compound No. | HCT116 (nM) | PC3 (nM) |
|---|---|---|
| 5 | 1.8 | 0.8 |
| 12 | 19 | 113 |
| 13 | 0.1 | 0.2 |
| 17 | 294 | 266 |
| 19 | 34 | 28 |
| 22 | 0.1 | 0.1 |
| 23 | 0.1 | 0.1 |
| 25 | 2.2 | 1.7 |
| 26 | 6.3 | 4.4 |
| 33 | 0.37 | 0.3 |
| 47 | 11 | 10 |
| 49 | 331 | 316 |
| 51 | 77 | 78 |
| 55 | 35 | 45 |
| 62 | 2.5 | 1.2 |
| 74 | 116 | 21 |
| 81 | 0.1 | 0.1 |
| 103 | 1.8 | 3 |
| 107 | 221 | 105 |
| 108 | 271 | 345 |
| 114 | 0.1 | 0.1 |

For the compounds in Table I, an IC₅₀<10 000 nM (10 μM) is found with regard to the HCT116 and PC3 lines. It is observed that some of the compounds exhibit an IC₅₀ value of <500 nM, some being very active with an IC₅₀ of 0.1 nM (cf. values in Table II). Thus, the compounds result in a loss of proliferation and viability of the tumour cells and therefore have an anticancer activity.

What is claimed is:

1. A compound of formula (I): 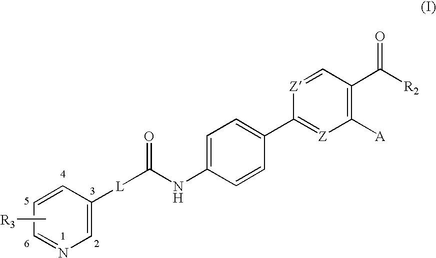
wherein:
A represents an —NR₁R′₁ or (C₁-C₆)alkoxy group;
Z and Z′ respectively represent N and CH; N and CF; N and N; CH and CH; CH and N;
L represents a —CH═CH— or —CH₂CH₂— or —(CH₂)ₙ—Y— group in which the Y group (attached to the C═O) represents an oxygen atom or an —NH— group and n is an integer ranging from 1 to 4;
R₁ and R′₁ are such that:
(i) R₁ represents:
a hydrogen atom;
an aryl group optionally substituted by one or more halogen atom(s);
a heteroaryl group;
a (C₃-C₆)cycloalkyl group;
a (C₁-C₆)alkyl group, optionally substituted by:
one or more hydroxyl or (C₁-C₆)alkoxy group(s);
an aryl group;
a (C₃-C₆)cycloalkyl group;
a heteroaryl group;
a heterocycloalkyl group;
an —NRₐR_{b} group in which Rₐ and R_{b} represent, independently of one another, a hydrogen atom or a (C₁-C₆)alkyl group or form, together with the nitrogen atom to which they are connected, a heterocycloalkyl group optionally comprising another nitrogen atom;
and R′₁ represents a hydrogen atom or a (C₁-C₆)alkyl group;
or
(ii) R₁ and R′₁ form, together with the nitrogen atom to which they are connected, a heterocycloalkyl group;
R₂ represents a -Q-R₄ group;
Q represents an oxygen atom or the —NH— group;
R₄ represents:
a hydrogen atom;
a heteroaryl group;
a (C₃-C₆)cycloalkyl group;
a (C₁-C₆)alkyl group, optionally substituted by:
one or more hydroxyl or (C₁-C₆)alkoxygroups;
a heteroaryl group;
a heterocycloalkyl group;
an —NR_{c}R_{d} group in which R_{c} and R_{d} represent, independently of one another, a hydrogen atom or a (C₁-C₆)alkyl group or form, together with the nitrogen atom to which they are connected, a heterocycloalkyl group optionally comprising, in the ring, another heteroatom, selected from a nitrogen or oxygen atom or the —S(O)_{q} group, with q=0, 1 or 2, and optionally being substituted by one or more substituent(s), which are identical to or different from one another when there are several of them, chosen from a halogen atom or an —OH; (C₁-C₄)alkoxy or (C₁-C₄)alkyl group; and
R₃ represents at least one substituent of the pyridine ring chosen from a hydrogen or fluorine atom or a (C₁-C₄)alkyl, (C₁-C₄)alkoxy, —OH, —CN or —NRₑR_{f} group in which Rₑ and R_{f} represent a hydrogen atom or a (C₁-C₄)alkyl group or else Rₑ represents a hydrogen atom and R_{f} represents a (C₁-C₄)alkyl, —C(═O)O(C₁-C₄)alkyl or —C(═O)(C₁-C₄)alkyl group;
or a salt thereof.

2. The compound according to claim 1 of formula: 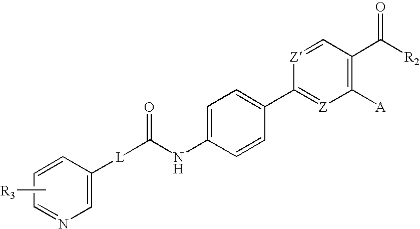
wherein:
A represents a (C₁-C₆)alkoxy group or an —NR₁R′₁ group;
Z and Z′ represent, independently of one another, N or CH;
L represents a —CH═CH— or —CH₂CH₂— or —(CH₂)ₙ—Y— group in which the Y group (attached to the C═O) represents an oxygen atom or an —NH— group and n is an integer ranging from 1 to 4;
R₁ and R′₁ are such that:
(i) R₁ represents:
a hydrogen atom;
an aryl group optionally substituted by one or more halogen atom(s);
a heteroaryl group;
a (C₃-C₆)cycloalkyl group;
a (C₁-C₆)alkyl group, optionally substituted by:
one or more hydroxyl or (C₁-C₆)alkoxygroup(s);
an aryl group;
a (C₃-C₆)cycloalkyl group;
a heteroaryl group;
a heterocycloalkyl group;
an —NRₐR_{b} group in which Rₐ and R_{b} represent, independently of one another, a hydrogen atom or a (C₁-C₆)alkylgroup or form, together with the nitrogen atom, a heterocycloalkyl group optionally comprising another nitrogen atom;
and R′₁ represents a hydrogen atom or a (C₁-C₆)alkyl group;
or
(ii) R₁ and R′₁ form, together with the nitrogen atom, a heterocycloalkyl group;
R₂ represents a (C₁-C₆)alkoxy group or an —NHR₄ group;
R₃ represents a hydrogen or fluorine atom or an —NH₂ group; and
R₄ represents:
a hydrogen atom;
a heteroaryl group;
a (C₃-C₆)cycloalkyl group;
a (C₁-C₆)alkyl group, optionally substituted by:
one or more hydroxyl or (C₁-C₆)alkoxygroups;
a heteroaryl group;
a heterocycloalkyl group;
an —NR_{c}R_{d} group in which R_{c} and R_{d} represent, independently of one another, a hydrogen atom or a (C₁-C₆)alkyl group or form, together with the nitrogen atom, a heterocycloalkyl group optionally comprising another nitrogen atom and optionally being substituted by one or more substituent(s), which are identical to or different from one another when there are several of them, chosen from: hydroxyl; (C₁-C₆)alkoxy; (C₁-C₆)alkyl; or a halogen atom;
or a salt thereof.

3. The compound according to claim 1, wherein R₁ is:
a phenyl group optionally substituted by a fluorine atom or the 3- or 4-pyridinyl; cyclopropyl; cyclobutyl; cyclopentyl; or cyclohexyl group;
a (C₁-C₆)alkyl group;
a (C₁-C₆)alkyl group substituted by one or more —OH or (C₁-C₄)alkoxy group(s);
a (C₁-C₆)alkyl group substituted by a phenyl; cyclopropyl; 2-, or 3-4-pyridinyl; or 2-tetrahydrofuryl group;
a (C₁-C₆)alkyl group substituted by the —NRₐR_{b} group in which Rₐ and R_{b} represent, independently of one another, a hydrogen atom or a (C₁-C₆)alkyl group or form, together with the nitrogen atom, a pyrrolidinyl, piperazinyl, piperidinyl or N—[(C₁-C₄)alkyl]piperidinyl group;
or a salt thereof.

4. The compound according to claim 3, wherein R₁ is selected from the group consisting of: 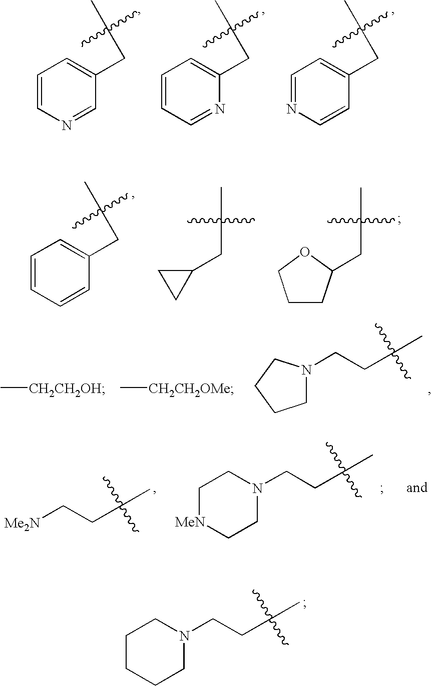
or a salt thereof.

5. The compound according to claim 1, wherein R₁ and R′₁ together form a pyrrolidinyl group; or a salt thereof.

6. The compound according to claim 1, wherein R₁ and R′₁ together form a piperidinyl or azetidinyl group; or a salt thereof.

7. The compound according to claim 1, wherein R₂ represents an —NHR₄ group in which R₄ represents:
a 3- or 4-pyridinyl, cyclopropyl or cyclopentyl group;
a (C₁-C₆)alkyl group;
a (C₁-C₆)alkyl group substituted by one or more —OH or (C₁-C₄)alkoxy group(s);
a (C₁-C₆)alkyl substituted by the 2-, 3- or 4-pyridinyl group;
a (C₁-C₆)alkyl group substituted by the morpholinyl, pyrrolidinyl, piperazinyl, piperidinyl or 4-N—[(C₁-C₄)alkyl]piperidinyl group;
a (C₁-C₆)alkyl group substituted by an —NR_{c}R_{d} group in which R_{c} and R_{d} represent, independently of one another, a hydrogen atom or a (C₁-C₆)alkyl group or form, together with the nitrogen atom to which they are connected, a pyrrolidinyl, piperidinyl, piperazinyl or N—[(C₁-C₄)alkyl]piperazinyl group optionally substituted by one or more substituent(s), which are identical or different when there are several of them, chosen from: —OH; (C₁-C₄)alkoxy; (C₁-C₄)alkyl; or a halogen atom;
or a salt thereof.

8. The compound according to claim 1, wherein R₂ represents an —NHR₄ group in which R₄ represents:
a 2-pyridinyl group;
a (C₁-C₆)alkyl group substituted by an —NR_{c}R_{d} group in which R_{c} and R_{d} represent, independently of one another, a hydrogen atom or a (C₁-C₆)alkyl group or form, together with the nitrogen atom to which they are connected, an azepanyl, morpholinyl, thiomorpholinyl, 1-oxothiomorpholinyl or 1,1-dioxothiomorpholinyl group;
or a salt thereof.

9. The compound according to claim 7, wherein the —NR_{c}R_{d} group is chosen from: 3-hydroxypiperidinyl, 4-hydroxypiperidinyl, 4,4′-difluoropiperidinyl, 4-methoxypiperidinyl, 2-methylpyrrolidinyl, cis-2,6-dimethylmorpholinyl and 3-fluoropyrrolidinyl;
or a salt thereof.

10. The compound according to claim 7, wherein R₂ is chosen from: 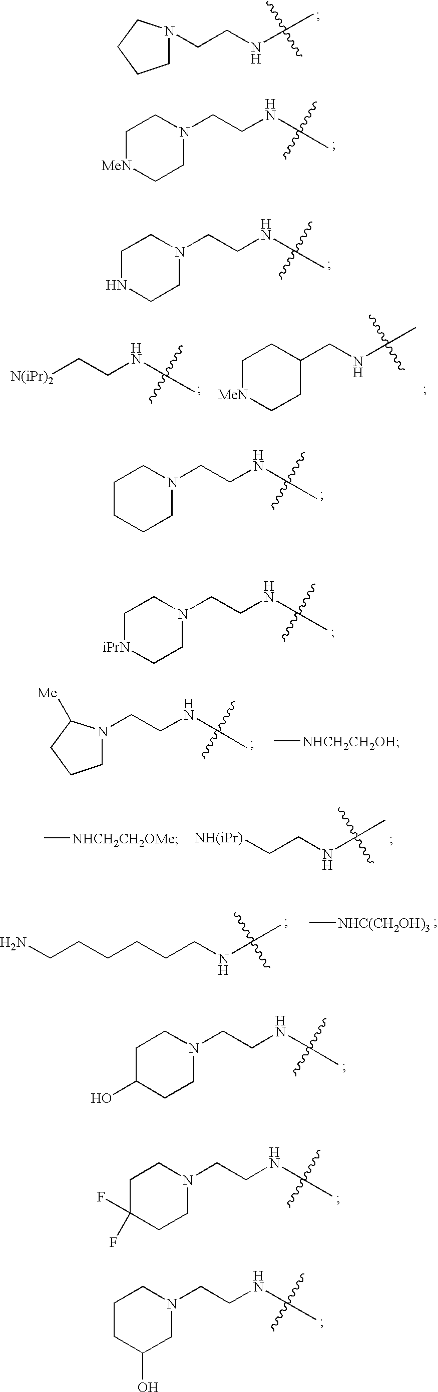 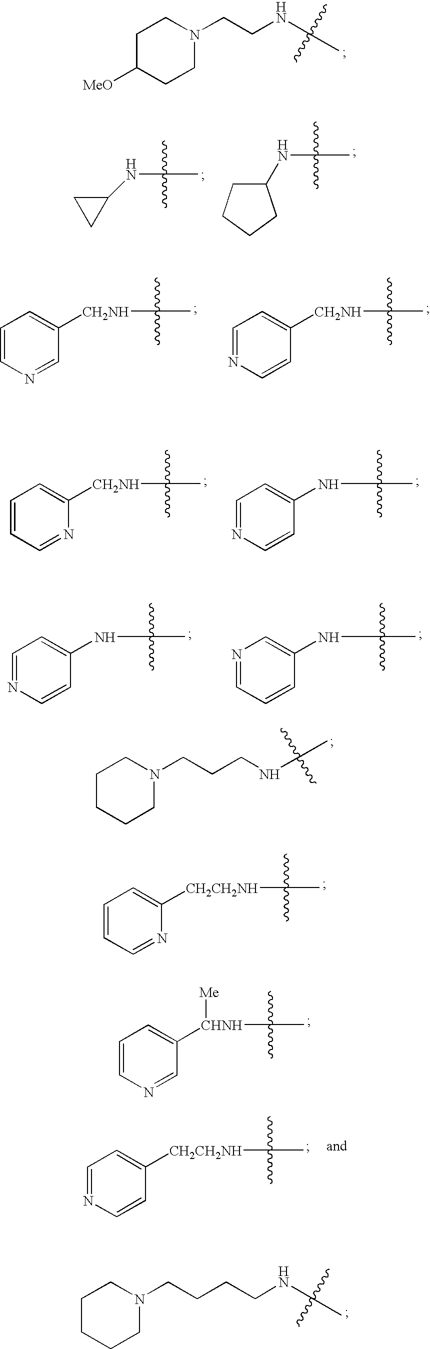
or a salt thereof.

11. The compound according to claim 7, wherein R₂ is chosen from: 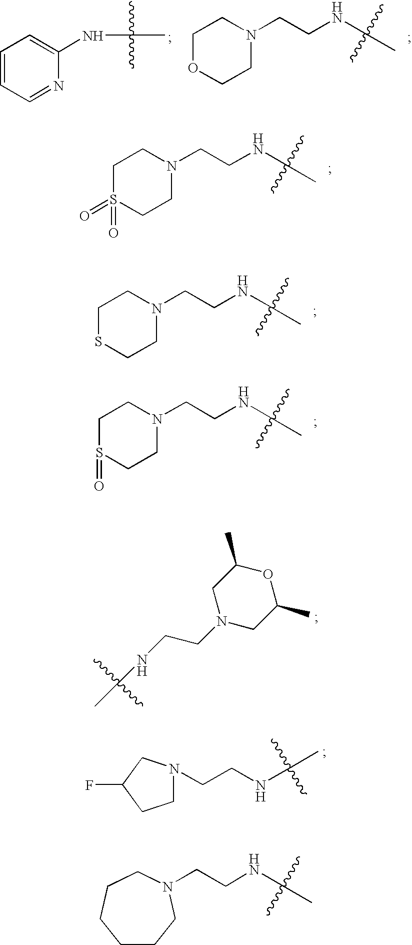
or a salt thereof.

12. The compound according to claim 1, wherein R₂ represents an —OR₄ group wherein R₄ represents a (C₁-C₄)alkyl group; or a salt thereof.

13. The compound according to claim 1, wherein R₂ represents and —OR₄ group wherein R₄ represents a (C₁-C₄)alkyl group substituted by the —NR_{c}R_{d} group in which R_{c} and R_{d} together form the piperidinyl group; or a salt thereof.

14. The compound according to claim 13, wherein R₂ represents 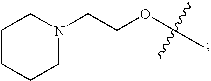
or a salt thereof.

15. The compound according to claim 1, wherein:
R₁ and R′₁ represent, independently of one another, a hydrogen atom or a (C₁-C₆)alkyl group;
Q represents the —NH— group; and
R₄ represents a hydrogen atom or a (C₁-C₆)alkyl group;
or a salt thereof.

16. The compound according to claim 15, wherein R₁ represents a (C₁-C₆)alkyl group and R′₁ represents a hydrogen atom or else R₁ and R′₁ represent two (C₁-C₆)alkyl groups; or a salt thereof.

17. The compound according to claim 1, wherein:
R₁ and R′₁ represent, independently of one another, a hydrogen atom or a (C₁-C₆)alkyl group;
Q represents the —NH— group; and
R₄ represents a (C₁-C₆)alkyl group substituted by:
one or more —OH or (C₁-C₆)alkoxy groups;
an —NR_{c}R_{d} group in which R_{c} and R_{d} represent, independently of one another, a hydrogen atom or a (C₁-C₆)alkyl group or form, together with the nitrogen atom to which they are connected, a heterocycloalkyl group chosen from a pyrrolidinyl, piperidinyl, piperazinyl or N—[(C₁-C₄)alkyl]piperazinyl, azepanyl, morpholinyl, thiomorpholinyl, 1-oxothiomorpholinyl, 1,1-dioxothiomorpholinyl, 3- or 4-hydroxypiperidinyl, 4,4′-difluoropiperidinyl, 4-methoxypiperidinyl, 2-methylpyrrolidinyl, cis-2,6-dimethylmorpholinyl or 3-fluoropyrrolidinyl group
or a salt thereof.

18. The compound according to claim 1, wherein:
R₁ represents a (C₁-C₆)alkyl group substituted by:
one or more —OH or (C₁-C₆)alkoxy group(s);
an —NRₐR_{b} group in which Rₐ and R_{b} represent, independently of one another, a hydrogen atom or a (C₁-C₆)alkyl group or form, together with the nitrogen atom to which they are connected, a heterocycloalkyl group chosen from a pyrrolidinyl, piperazinyl, piperidinyl or N—[(C₁-C₄)alkyl]piperidinyl group;
R′₁ represents a hydrogen atom;
Q represents the —NH— group; and
R₄ represents a (C₁-C₆)alkyl group;
or a salt thereof.

19. The compound according to claim 1, wherein:
R₁ represents a (C₁-C₆)alkyl group substituted by a phenyl or 2-, 3- or 4-pyridinyl group;
R′₁ represents a hydrogen atom;
Q represents the —NH— group; and
R₄ represents a (C₁-C₆)alkyl group;
or a salt thereof.

20. The compound according claim 1, wherein:
R₁ represents a (C₃-C₆)cycloalkyl group;
R′₁ represents a hydrogen atom;
Q represents the —NH— group; and
R₄ represents a (C₁-C₆)alkyl group or a (C₃-C₆)cycloalkyl group;
or a salt thereof.

21. The compound according to claim 1, wherein:
R₁ represents a phenyl or 3- or 4-pyridinyl group;
R′₁ represents a hydrogen atom;
Q represents the —NH— group; and
R₄ represents a (C₁-C₆)alkyl group;
or a salt thereof.

22. The compound according to claim 1, wherein:
R₁ represents a phenyl group optionally substituted by one or more halogen atom(s);
R′₁ represents a hydrogen atom;
Q represents the —NH— group; and
R₄ represents a (C₁-C₆)alkyl group optionally substituted by the —NR_{c}R_{d} group in which R_{c} and R_{d} form, together with the nitrogen atom to which they are connected, a heterocycloalkyl group chosen from the pyrrolidinyl or piperidinyl group;
or a salt thereof.

23. The compound according to claim 1, wherein:
R₁ and R′₁ represent, independently of one another, a hydrogen atom or a (C₁-C₆)alkyl group;
Q represents the —NH— group;
R₄ represents a (C₁-C₆)alkyl group substituted by a 2-, 3- or 4-pyridinyl group;
or a salt thereof.

24. The compound according to claim 1 of formula (I′): 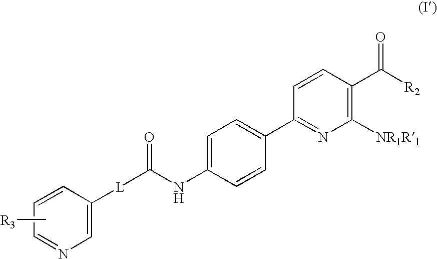
wherein R₁, R′₁, R₂, L and R₃ are as defined in claim 1;
or a salt thereof.

25. The compound according to claim 1 of formula (I″): 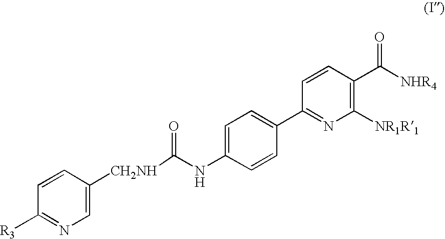
in which R₁, R′₁, R₃ and R₄ are as defined in claim 1;
or a salt thereof.

26. The compound according to claim 1, wherein L represents a —CH₂NH—, —CH₂O—, —CH₂CH₂— or —CH═CH— group;
or a salt thereof.

27. The compound according to claim 1, wherein R₃ represents a hydrogen atom or an —NH₂ group;
or a salt thereof.

28. The compound according to claim 1 selected from the group consisting of:
2-Ethylamino-N-methyl-6-[4-(3-pyridin-3-ylmethyl-ureido)-phenyl]-nicotinamide;
2-Ethylamino-6-[4-(3-pyridin-3-ylmethyl-ureido)-phenyl]-N-(2-pyrrolidin-1-yl-ethyl)-nicotinamide;
2-Amino-N-methyl-6-[4-(3-pyridin-3-ylmethyl-ureido)-phenyl]-nicotinamide;
2-Ethylamino-N-[2-(4-methyl-piperazin-1-yl)-ethyl]-6-[4-(3-pyridin-3-ylmethyl-ureido)-phenyl]-nicotinamide;
2-Ethylamino-N-(2-piperazin-1-yl-ethyl)-6-[4-(3-pyridin-3-ylmethyl-ureido)-phenyl]-nicotinamide;
N-Methyl-6-[4-(3-pyridin-3-ylmethyl-ureido)-phenyl]-2-(2-pyrrolidin-1-yl-ethylamino)-nicotinamide;
2-(2-Dimethylamino-ethylamino)-N-methyl-6-[4-(3-pyridin-3-ylmethyl-ureido)-phenyl]-nicotinamide;
N-(2-Diisopropylamino-ethyl)-2-ethylamino-6-[4-(3-pyridin-3-ylmethyl-ureido)-phenyl]-nicotinamide;
N-(2-Dimethylamino-ethyl)-2-ethylamino-6-[4-(3-pyridin-3-ylmethyl-ureido)-phenyl]-nicotinamide;
2-Ethylamino-N-(1-methyl-piperidin-4-ylmethyl)-6-[4-(3-pyridin-3-ylmethyl-ureido)-phenyl]-nicotinamide;
N-Methyl-2-[2-(4-methyl-piperazin-1-yl)-ethylamino]-6-[4-(3-pyridin-3-ylmethyl-ureido)-phenyl]-nicotinamide;
N-Methyl-2-[(pyridin-3-ylmethyl)-amino]-6-[4-(3-pyridin-3-ylmethyl-ureido)-phenyl]-nicotinamide;
2-Ethylamino-N-(2-piperidin-1-yl-ethyl)-6-[4-(3-pyridin-3-ylmethyl-ureido)-phenyl]-nicotinamide;
N-Methyl-2-[(pyridin-2-ylmethyl)-amino]-6-[4-(3-pyridin-3-ylmethyl-ureido)-phenyl]-nicotinamide;
N-Methyl-2-[(pyridin-4-ylmethyl)-amino]-6-[4-(3-pyridin-3-ylmethyl-ureido)-phenyl]-nicotinamide;
2-Ethylamino-6-[4-(3-pyridin-3-ylmethyl-ureido)-phenyl]-nicotinamide;
N-Methyl-2-(2-piperidin-1-yl-ethylamino)-6-[4-(3-pyridin-3-ylmethyl-ureido)-phenyl]-nicotinamide;
2-Ethylamino-N-[2-(4-isopropyl-piperazin-1-yl)-ethyl]-6-[4-(3-pyridin-3-ylmethyl-ureido)-phenyl]-nicotinamide;
2-Benzylamino-N-methyl-6-[4-(3-pyridin-3-ylmethyl-ureido)-phenyl]-nicotinamide;
2-Ethylamino-N-[2-(2-methyl-pyrrolidin-1-yl)-ethyl]-6-[4-(3-pyridin-3-ylmethyl-ureido)-phenyl]-nicotinamide;
6-{4-[3-(6-Amino-pyridin-3-ylmethyl)-ureido]-phenyl}-2-ethylamino-N-methyl-nicotinamide;
N-Methyl-2-phenylamino-6-[4-(3-pyridin-3-ylmethyl-ureido)-phenyl]-nicotinamide;
2-Cyclopropylamino-N-methyl-6-[4-(3-pyridin-3-ylmethyl-ureido)-phenyl]-nicotinamide;
2-Amino-6-[4-(3-pyridin-3-ylmethyl-ureido)-phenyl]-nicotinamide;
2-Diethylamino-N-methyl-6-[4-(3-pyridin-3-ylmethyl-ureido)-phenyl]-nicotinamide;
2-Ethylamino-N-(2-hydroxy-ethyl)-6-[4-(3-pyridin-3-ylmethyl-ureido)-phenyl]-nicotinamide;
2-Ethylamino-N-(2-methoxy-ethyl)-6-[4-(3-pyridin-3-ylmethyl-ureido)-phenyl]-nicotinamide;
2-Ethylamino-6-[4-(3-pyridin-3-ylmethyl-ureido)-phenyl]-nicotinic acid ethyl ester;
[4-(6-Ethylamino-5-methylcarbamoyl-pyridin-2-yl)-phenyl]-carbamic acid pyridin-3-ylmethyl ester;
2-Ethylamino-N-methyl-6-{4-[3-(2-pyridin-3-yl-ethyl)-ureido]-phenyl}-nicotinamide;
2-Ethylamino-N-(2-isopropylamino-ethyl)-6-[4-(3-pyridin-3-ylmethyl-ureido)-phenyl]-nicotinamide;
N-(6-Amino-hexyl)-2-ethylamino-6-[4-(3-pyridin-3-ylmethyl-ureido)-phenyl]-nicotinamide;
2-Phenylamino-6-[4-(3-pyridin-3-ylmethyl-ureido)-phenyl]-N-(2-pyrrolidin-1-yl-ethyl)-nicotinamide;
2-Ethylamino-N-(2-hydroxy-1,1-bis-hydroxymethyl-ethyl)-6-[4-(3-pyridin-3-ylmethyl-ureido)-phenyl]-nicotinamide;
2-Isopropylamino-N-methyl-6-[4-(3-pyridin-3-ylmethyl-ureido)-phenyl]-nicotinamide;
2-Cyclohexylamino-N-methyl-6-[4-(3-pyridin-3-ylmethyl-ureido)-phenyl]-nicotinamide;
2-Cyclopentylamino-N-methyl-6-[4-(3-pyridin-3-ylmethyl-ureido)-phenyl]-nicotinamide;
2-Cyclobutylamino-N-methyl-6-[4-(3-pyridin-3-ylmethyl-ureido)-phenyl]-nicotinamide;
2-Phenylamino-N-(2-piperidin-1-yl-ethyl)-6-[4-(3-pyridin-3-ylmethyl-ureido)-phenyl]-nicotinamide;
2-Ethylamino-N-[2-(4-hydroxy-piperidin-1-yl)-ethyl]-6-[4-(3-pyridin-3-ylmethyl-ureido)-phenyl]-nicotinamide;
N-[2-(4,4-Difluoro-piperidin-1-yl)-ethyl]-2-ethylamino-6-[4-(3-pyridin-3-ylmethyl-ureido)-phenyl]-nicotinamide;
2-Ethylamino-N-[2-(3-hydroxy-piperidin-1-yl)-ethyl]-6-[4-(3-pyridin-3-ylmethyl-ureido)-phenyl]-nicotinamide;
2-Ethylamino-N-[2-(4-methoxy-piperidin-1-yl)-ethyl]-6-[4-(3-pyridin-3-ylmethyl-ureido)-phenyl]-nicotinamide;
2-(3-Fluoro-phenylamino)-N-(2-piperidin-1-yl-ethyl)-6-[4-(3-pyridin-3-ylmethyl-ureido)-phenyl]-nicotinamide;
2-(4-Fluoro-phenylamino)-N-(2-piperidin-1-yl-ethyl)-6-[4-(3-pyridin-3-ylmethyl-ureido)-phenyl]-nicotinamide;
2-(2-Fluoro-phenylamino)-N-(2-piperidin-1-yl-ethyl)-6-[4-(3-pyridin-3-ylmethyl-ureido)-phenyl]-nicotinamide;
4-Ethylamino-2-[4-(3-pyridin-3-ylmethyl-ureido)-phenyl]-pyrimidine-5-carboxylic acid methylamide;
2-(Cyclopropylmethyl-amino)-N-methyl-6-[4-(3-pyridin-3-ylmethyl-ureido)-phenyl]-nicotinamide;
N-Methyl-6-[4-(3-pyridin-3-ylmethyl-ureido)-phenyl]-2-pyrrolidin-1-yl-nicotinamide;
N-Methyl-6-[4-(3-pyridin-3-ylmethyl-ureido)-phenyl]-2-[(tetrahydro-furan-2-ylmethyl)-amino]-nicotinamide;
2-(2-Methoxy-ethylamino)-N-methyl-6-[4-(3-pyridin-3-ylmethyl-ureido)-phenyl]-nicotinamide;
2-(2-Hydroxy-ethylamino)-N-methyl-6-[4-(3-pyridin-3-ylmethyl-ureido)-phenyl]-nicotinamide;
N-Methyl-2-(pyridin-3-ylamino)-6-[4-(3-pyridin-3-ylmethyl-ureido)-phenyl]-nicotinamide;
N-Methyl-2-(pyridin-4-ylamino)-6-[4-(3-pyridin-3-ylmethyl-ureido)-phenyl]-nicotinamide;
4-Ethylamino-N-methyl-6-[4-(3-pyridin-3-ylmethyl-ureido)-phenyl]-nicotinamide;
2-Ethylamino-N-methyl-6-[4-(3-pyridin-3-yl-propionylamino)-phenyl]-nicotinamide;
2-Cyclopropylamino-N-(2-piperidin-1-yl-ethyl)-6-[4-(3-pyridin-3-ylmethyl-ureido)-phenyl]-nicotinamide;
N-Cyclopropyl-2-cyclopropylamino-6-[4-(3-pyridin-3-ylmethyl-ureido)-phenyl]-nicotinamide;
N-Butyl-2-cyclopropylamino-6-[4-(3-pyridin-3-ylmethyl-ureido)-phenyl]-nicotinamide;
N-Cyclopentyl-2-cyclopropylamino-6-[4-(3-pyridin-3-ylmethyl-ureido)-phenyl]-nicotinamide;
2-Cyclopropylamino-N-ethyl-6-[4-(3-pyridin-3-ylmethyl-ureido)-phenyl]-nicotinamide;
3-Ethylamino-4′-(3-pyridin-3-ylmethyl-ureido)-biphenyl-4-carboxylic acid methylamide;
2-Ethoxy-N-methyl-6-[4-(3-pyridin-3-ylmethyl-ureido)-phenyl]-nicotinamide;
2-Ethylamino-N-pyridin-3-ylmethyl-6-[4-(3-pyridin-3-ylmethyl-ureido)-phenyl]-nicotinamide;
2-Ethylamino-N-pyridin-4-ylmethyl-6-[4-(3-pyridin-3-ylmethyl-ureido)-phenyl]-nicotinamide;
2-Ethylamino-N-pyridin-2-ylmethyl-6-[4-(3-pyridin-3-ylmethyl-ureido)-phenyl]-nicotinamide;
2-Ethylamino-N-pyridin-4-yl-6-[4-(3-pyridin-3-ylmethyl-ureido)-phenyl]-nicotinamide;
2-Ethylamino-N-pyridin-3-yl-6-[4-(3-pyridin-3-ylmethyl-ureido)-phenyl]-nicotinamide;
2-Ethylamino-N-(3-piperidin-1-yl-propyl)-6-[4-(3-pyridin-3-ylmethyl-ureido)-phenyl]-nicotinamide;
2-Ethylamino-N-(2-pyridin-2-yl-ethyl)-6-[4-(3-pyridin-3-ylmethyl-ureido)-phenyl]-nicotinamide;
2-Ethylamino-N-(1-pyridin-3-yl-ethyl)-6-[4-(3-pyridin-3-ylmethyl-ureido)-phenyl]-nicotinamide;
2-Ethylamino-N-(2-pyridin-4-yl-ethyl)-6-[4-(3-pyridin-3-ylmethyl-ureido)-phenyl]-nicotinamide;
2-Ethylamino-N-methyl-6-[4-((E)-3-pyridin-3-yl-acryloylamino)-phenyl]-nicotinamide;
N-(2-Diisopropylamino-ethyl)-2-ethylamino-6-[4-((E)-3-pyridin-3-yl-acryloylamino)-phenyl]-nicotinamide;
2-Ethylamino-N-(2-piperidin-1-yl-ethyl)-6-[4-((E)-3-pyridin-3-yl-acryloylamino)-phenyl]-nicotinamide;
2-Ethylamino-N-(4-piperidin-1-yl-butyl)-6-[4-(3-pyridin-3-ylmethyl-ureido)-phenyl]-nicotinamide;
2-Ethylamino-N-pyridin-2-yl-6-[4-(3-pyridin-3-ylmethyl-ureido)-phenyl]-nicotinamide;
2-Ethylamino-5-fluoro-N-methyl-6-[4-(3-pyridin-3-ylmethyl-ureido)-phenyl]-nicotinamide;
2-Ethylamino-5-fluoro-N-(2-piperidin-1-yl-ethyl)-6-[4-(3-pyridin-3-ylmethyl-ureido)-phenyl]-nicotinamide;
4-Ethylamino-2-[4-(3-pyridin-3-ylmethyl-ureido)-phenyl]-pyrimidine-5-carboxylic acid (2-piperidin-1-yl-ethyl)-amide;
6-{4-[3-(6-Amino-pyridin-3-ylmethyl)-ureido]-phenyl}-2-ethylamino-N-(2-piperidin-1-yl-ethyl)-nicotinamide;
2-Ethylamino-6-{-4-[3-(2-fluoro-pyridin-3-ylmethyl)-ureido]-phenyl}-N-(2-piperidin-1-yl-ethyl)-nicotinamide;
2-Ethylamino-6-{-4-[3-(6-methyl-pyridin-3-ylmethyl)-ureido]-phenyl}-N-(2-piperidin-1-yl-ethyl)-nicotinamide
2-Ethylamino-N-(2-piperidin-1-yl-ethyl)-6-{-4-[3-(2,5,6-trifluoro-pyridin-3-ylmethyl)-ureido]-phenyl}-nicotinamide;
2-Ethylamino-6-{-4-[3-(5-methyl-pyridin-3-ylmethyl)-ureido]-phenyl}-N-(2-piperidin-1-yl-ethyl)-nicotinamide;
2-Ethylamino-6-{-4-[3-(2-methoxy-pyridin-3-ylmethyl)-ureido]-phenyl}-N-(2-piperidin-1-yl-ethyl)-nicotinamide;
6-{4-[3-(5-Amino-pyridin-3-ylmethyl)-ureido]-phenyl}-2-ethylamino-N-(2-piperidin-1-yl-ethyl)-nicotinamide;
2-Ethylamino-6-{-4-[3-(5-fluoro-pyridin-3-ylmethyl)-ureido]-phenyl}-N-(2-piperidin-1-yl-ethyl)-nicotinamide;
2-Ethylamino-6-{-4-[3-(6-fluoro-pyridin-3-ylmethyl)-ureido]-phenyl}-N-(2-piperidin-1-yl-ethyl)-nicotinamide;
6-{4-[3-(6-Dimethylamino-pyridin-3-ylmethyl)-ureido]-phenyl}-2-ethylamino-N-(2-piperidin-1-yl-ethyl)-nicotinamide;
6-{4-[3-(6-Cyano-pyridin-3-ylmethyl)-ureido]-phenyl}-2-ethylamino-N-(2-piperidin-1-yl-ethyl)-nicotinamide;
6-{4-[3-(6-tert-Butoxycarbonylamino-pyridin-3-ylmethyl)-ureido]-phenyl}-2-ethylamino-nicotinic acid 2-piperidin-1-yl-ethyl ester;
6-{4-[3-(6-Amino-pyridin-3-ylmethyl)-ureido]-phenyl}-2-ethylamino-nicotinic acid 2-piperidin-1-yl-ethyl ester;
2-Ethylamino-6-{-4-[3-(6-methylamino-pyridin-3-ylmethyl)-ureido]-phenyl}-N-(2-piperidin-1-yl-ethyl)-nicotinamide;
6-{4-[3-(6-Amino-5-methyl-pyridin-3-ylmethyl)-ureido]-phenyl}-2-ethylamino-N-(2-piperidin-1-yl-ethyl)-nicotinamide;
6-{-4-[3-(6-Amino-pyridin-3-ylmethyl)-ureido]-phenyl}-2-ethylamino-N-(2-morpholin-4-yl-ethyl)-nicotinamide;
6-{-4-[3-(6-Amino-pyridin-3-ylmethyl)-ureido]-phenyl}-N-[2-(1,1-dioxo-1-thiomorpholin-4-yl)-ethyl]-2-ethylamino-nicotinamide;
6-{-4-[3-(6-Amino-pyridin-3-ylmethyl)-ureido]-phenyl}-2-phenylamino-N-(2-piperidin-1-yl-ethyl)-nicotinamide;
6-{-4-[3-(6-Amino-pyridin-3-ylmethyl)-ureido]-phenyl}-2-cyclopropylamino-N-(2-piperidin-1-yl-ethyl)-nicotinamide;
6-{-4-[3-(6-Amino-pyridin-3-ylmethyl)-ureido]-phenyl}-2-ethylamino-N-(2-thiomorpholin-4-yl-ethyl)-nicotinamide;
6-{-4-[3-(6-Acetylamino-pyridin-3-ylmethyl)-ureido]-phenyl}-2-ethylamino-N-(2-piperidin-1-yl-ethyl)-nicotinamide;
6-{4-[(E)-3-(6-Amino-pyridin-3-yl)-acryloylamino]-phenyl}-2-ethylamino-N-(2-piperidin-1-yl-ethyl)-nicotinamide;
6-{-4-[3-(6-Amino-pyridin-3-ylmethyl)-ureido]-phenyl}-2-ethylamino-N-[2-(1-oxo-1-thiomorpholin-4-yl)-ethyl]-nicotinamide;
6-{-4-[3-(6-Amino-pyridin-3-ylmethyl)-ureido]-phenyl}-2-ethylamino-N-(2-isopropylamino-ethyl)-nicotinamide;
4′-[3-(6-Amino-pyridin-3-ylmethyl)-ureido]-3-ethylamino-biphenyl-4-carboxylic acid (2-piperidin-1-yl-ethyl)-amide;
4′-[3-(6-Amino-5-methyl-pyridin-3-ylmethyl)-ureido]-3-ethylamino-biphenyl-4-carboxylic acid (2-piperidin-1-yl-ethyl)amide;
2-Ethylamino-6-{-4-[3-(6-isobutyrylamino-pyridin-3-ylmethyl)-ureido]-phenyl}-N-(2-piperidin-1-yl-ethyl)-nicotinamide;
2-Ethylamino-6-{-4-[3-(6-isopropylamino-pyridin-3-ylmethyl)-ureido]-phenyl}-N-(2-piperidin-1-yl-ethyl)-nicotinamide;
2-Ethylamino-6-{-4-[3-(6-ethylamino-pyridin-3-ylmethyl)-ureido]-phenyl}-N-(2-piperidin-1-yl-ethyl)-nicotinamide;
{5-[3-(4-{5-[2-(1,1-Dioxo-1-thiomorpholin-4-yl)-ethylcarbamoyl]-6-ethylamino-pyridin-2-yl}-phenyl)-ureidomethyl]-pyridin-2-yl}-carbamic acid tert-butyl ester;
{5-[3-(4-{5-[2-(cis-2,6-Dimethyl-morpholin-4-yl)-ethylcarbamoyl]-6-ethylamino-pyridin-2-yl}-phenyl)-ureidomethyl]-pyridin-2-yl}-carbamic acid tert-butyl ester;
6-{4-[3-(6-Amino-pyridin-3-ylmethyl)-ureido]-phenyl}-N-cyclopropyl-2-cyclopropylamino-nicotinamide;
6-{4-[3-(6-Amino-pyridin-3-ylmethyl)-ureido]-phenyl}-N-butyl-2-cyclopropylamino-nicotinamide;
6-{-4-[3-(6-Amino-pyridin-3-ylmethyl)-ureido]-phenyl}-N-[2-(cis-2,6-dimethyl-morpholin-4-yl)-ethyl]-2-ethylamino-nicotinamide;
6-{-4-[3-(6-Amino-pyridin-3-ylmethyl)-ureido]-phenyl}-2-ethylamino-N-(2-hydroxy-ethyl)-nicotinamide;
6-{-4-[3-(6-Amino-pyridin-3-ylmethyl)-ureido]-phenyl}-2-azetidin-1-yl-N-(2-piperidin-1-yl-ethyl)-nicotinamide;
6-{-4-[3-(6-Amino-pyridin-3-ylmethyl)-ureido]-phenyl}-N-cyclopentyl-2-cyclopropylamino-nicotinamide;
6-{-4-[3-(6-Amino-pyridin-3-ylmethyl)-ureido]-phenyl}-2-cyclopropylamino-N-ethyl-nicotinamide;
4′-[3-(6-Amino-pyridin-3-ylmethyl)-ureido]-3-cyclopropylamino-biphenyl-4-carboxylic acid (2-piperidin-1-yl-ethyl)-amide;
6-{-4-[3-(6-Amino-pyridin-3-ylmethyl)-ureido]-phenyl}-2-ethylamino-N-(2-methoxy-ethyl)-nicotinamide;
6-{-4-[3-(6-Amino-pyridin-3-ylmethyl)-ureido]-phenyl}-N-(2-azepan-1-yl-ethyl)-2-ethylamino-nicotinamide;
6-{-4-[3-(6-Amino-pyridin-3-ylmethyl)-ureido]-phenyl}-2-ethylamino-N-(2-pyrrolidin-1-yl-ethyl)-nicotinamide;
2-Ethylamino-6-{-4-[3-(6-oxo-1,6-dihydro-pyridin-3-ylmethyl)-ureido]-phenyl}-N-(2-piperidin-1-yl-ethyl)-nicotinamide;
6-{-4-[3-(2-Amino-pyridin-3-ylmethyl)-ureido]-phenyl}-2-ethylamino-N-(2-piperidin-1-yl-ethyl)-nicotinamide;
6-{-4-[3-(6-Amino-pyridin-3-ylmethyl)-ureido]-phenyl}-2-ethylamino-N-[2-(3-fluoro-pyrrolidin-1-yl)-ethyl]-nicotinamide;
2-{-4-[3-(6-Amino-pyridin-3-ylmethyl)-ureido]-phenyl}-4-ethylamino-pyrimidine-5-carboxylic acid (2-piperidin-1-yl-ethyl)amide;
2-{-4-[3-(6-Amino-pyridin-3-ylmethyl)-ureido]-phenyl}-4-cyclopropylamino-pyrimidine-5-carboxylic acid (2-piperidin-1-yl-ethyl)amide;
6-{-4-[3-(6-Amino-pyridin-3-ylmethyl)-ureido]-phenyl}-N-(2-piperidin-1-yl-ethyl)-2-pyrrolidin-1-yl-nicotinamide; and
6′-{-4-[3-(6-Amino-pyridin-3-ylmethyl)-ureido]-phenyl}-3,4,5,6-tetrahydro-2H-[1,2′]bipyridinyl-3′-carboxylic acid (2-piperidin-1-yl-ethyl)amide;
or a salt thereof.

29. A process for the preparation of a compound of formula: 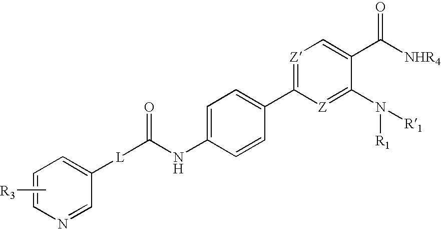
comprising coupling, in the presence of a palladium complex and optionally of a base, the compound of formula 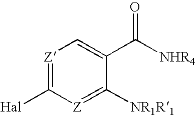
with the compound of formula 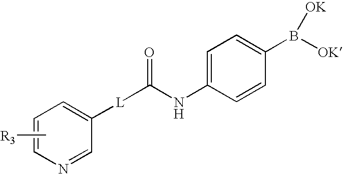
wherein R₁, R′₁, R₃, R₄, L, Z and Z′ are as defined in claim 1, Hal represents a halogen atom and K and K′ represent a hydrogen atom or an alkyl or aryl group, optionally connected to one another in order to form, together with the boron atom and the two oxygen atoms, a 5- to 7-membered ring.

30. A process for the preparation of a compound of formula: 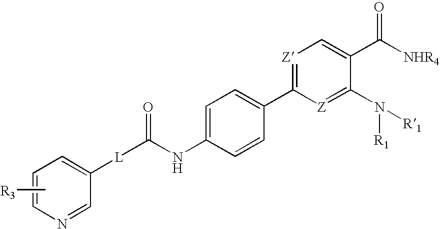
comprising reacting a compound of formula: 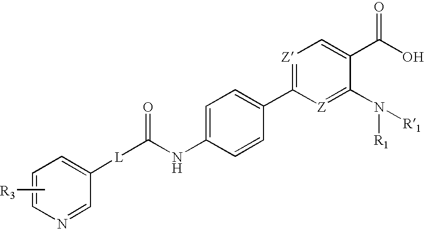
with R₄NH₂, optionally in the presence of an acid activator, wherein R₁, R′₁, R₃, R₄, L, Z and Z′ are as defined in claim 1.

31. A process for the preparation of a compound of formula: 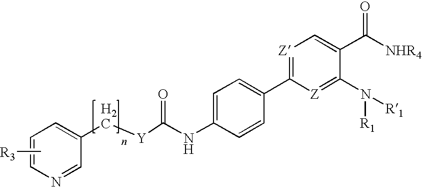
comprising reacting a compound of formula 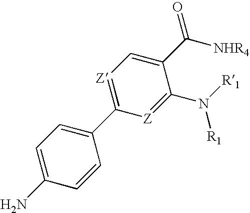
with the compound P₄ of formula 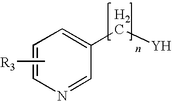
in the presence of an agent which makes it possible to introduce the “C═O” unit and optionally of a base, wherein R₁, R′₁, R₃, R₄, L, Z, Z′ and n are as defined in claim 1.

32. A process for the preparation of a compound of formula: 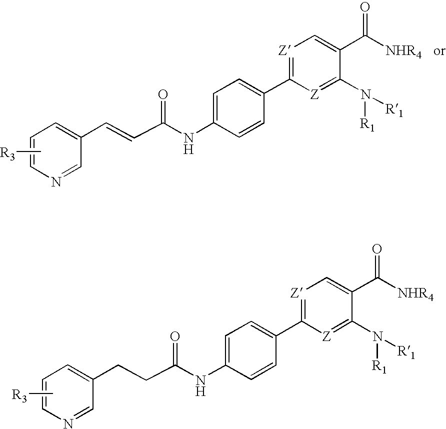
comprising respectively reacting the compound of formula 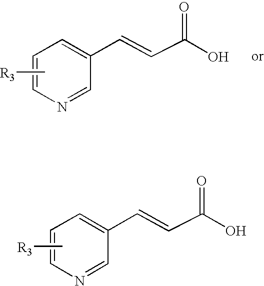
with the compound of formula 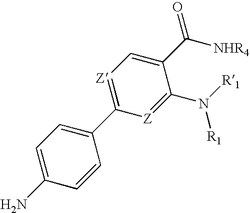
optionally in the presence of an acid activator, wherein R₁, R′₁, R₃, R₄, Z and Z′ are as defined in one of claim 1.

33. A compound of formula: 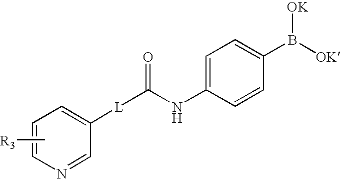
wherein L represents a —CH═CH— or —CH₂CH₂— or —(CH₂)ₙ—Y— group in which the Y group (attached to the C═O) represents an oxygen atom or an —NH— group and n is an integer ranging from 1 to 4, R₃ is as defined in claim 1 and K and K′ represent a hydrogen atom or an alkyl or aryl group, optionally connected to one another in order to form, together with the boron atom and the two oxygen atoms, a 5- to 7-membered ring.

34. A pharmaceutical composition comprising a compound according to claim 1, or a pharmaceutically acceptable salt thereof and at least one pharmaceutically acceptable excipient.

35. A method of treating cancer comprising administering to a patient in need thereof an effective amount of a compound according to claim 1.
